(19) United States
(12) Patent Application Publication
(10) Pub. No.: US 2020/0172597 A1
(43) Pub. Date: Jun. 4, 2020

(54) ARTIFICIAL ANTIGEN PRESENTING CELLS INCLUDING HLA-E AND HLA-G MOLECULES AND METHODS OF USE

(71) Applicant: Rubius Therapeutics, Inc., Cambridge, MA (US)

(72) Inventors: Tiffany Fen-Yi Chen, Cambridge, MA (US); Thomas Joseph Wickham, Groton, MA (US); Regina Sophia Salvat, Cambridge, MA (US); Xuqing Zhang, Quincy, MA (US)

(21) Appl. No.: 16/702,249

(22) Filed: Dec. 3, 2019

Related U.S. Application Data
(60) Provisional application No. 62/774,857, filed on Dec. 3, 2018.

(51) Int. Cl. C07K 14/74; C12N 5/078; C07K 14/47; A61P 37/02

(52) U.S. Cl. CPC C07K 14/70539; C12N 5/0641; A61K 38/00; A61P 37/02; C07K 14/4747

(57) ABSTRACT

The present disclosure relates to artificial antigen presenting cells (aAPCs), in particular engineered erythroid cells and enucleated cells (e.g., enucleated erythroid cells and platelets), that are engineered to include exogenous-antigen presenting polypeptides comprising either HLA-E or HLA-G polypeptides, which may be used to activate or suppress certain immune cells.

### RELATED APPLICATIONS

This application claims the benefit of priority to U.S. Provisional Patent Application No. 62/774,857, filed Dec. 3, 2018. The contents of this application are incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 3, 2019, is named 129267-01002_SL.txt and is 34,381 bytes in size.

### BACKGROUND

Antigen-presenting cells (APCs) act as a link between the innate and adaptive immune responses. Upon internalization of an antigen, the APCs can display antigens (or peptides derived therefrom) on class I and II major histocompatibility complex (MHC) on the cell membrane, together with costimulatory signals, to regulate immune cells (e.g., activate antigen-specific T cells). The activation and/or expansion of antigen-specific immune cells (e.g., T cells) may be desirable to treat some diseases, such as cancer. For example, populations of antigen-specific T cells can be primed and amplified ex vivo to obtain a large number of tumor-specific T cells for transfusion into a subject. The activation and/or expansion of desirable antigen-specific immune cells may be accomplished by administering APCs that are generated ex vivo. For example, dendritic cells (DCs) have been used to maximize T cell (e.g., CD8+ cytotoxic lymphocytes (CTL)) stimulation ex vivo. However, the preparation of DCs ex vivo for clinical use remains challenging (Kim J V et al. Nat Biotechnol. 2004; 22: 403-10).

Multiple systems, including synthetic biomaterials, have been engineered to activate and/or expand desirable immune cell populations (e.g., T cells). These systems may act by mimicking the interaction between DCs and T cells. For instance, several cell-sized, rigid, beads, such as latex microbeads, polystyrene-coated magnetic microbeads and biodegradable poly(lactic-co-glycolic acid) microparticles, have been developed. The efficacy of these beads in inducing activation and/or expansion of immune cells appears to be highly dependent on the properties of the materials used. For example, beads greater than 200 nm are typically retained at the site of inoculation, while smaller particles may be taken up by DCs (see, e.g., Reddy et Al. (2006) J. Control. Release 112: 26-34). In contrast, the membrane of natural APCs is much more dynamic than the outer surface of these beads. Thus, there remains a need for improved compositions and methods for stimulating desirable immune cells for therapeutic purposes. Preferably, these compositions are autologous and exhibit prolonged clearance rate in the subject to whom they are administered to activate and/or induce a desired immune cell population.

### SUMMARY

The present disclosure relates to artificial antigen presenting cells (aAPCs), in particular engineered erythroid cells (e.g., engineered enucleated erythroid cells) or enucleated cells (e.g., modified enucleated cells), that are engineered to include an HLA-E or HLA-G polypeptide. In some embodiments, the aAPCs further comprise an exogenous antigenic polypeptide bound to the HLA-E or HLA-G polypeptide. In some embodiments, the engineered erythroid cells are engineered enucleated erythroid cells, e.g., reticulocytes or erythrocytes. In some embodiments of the present disclosure, the enucleated cell (e.g., modified enucleated cell) is a reticulocyte, an erythrocyte or a platelet.

In a first aspect, the disclosure features an artificial antigen presenting cell (aAPC) comprising an engineered enucleated erythroid cell comprising an exogenous antigen-presenting polypeptide on the cell surface, wherein the exogenous antigen-presenting polypeptide comprises a human leukocyte antigen-E (HLA-E) polypeptide, and an exogenous antigenic polypeptide that is specifically bound to the exogenous antigen-presenting polypeptide.

In some embodiments, the HLA-E polypeptide comprises an allele selected from the group consisting of: E*01:01:01:01, E*01:01:01:02, E*01:01:01:03, E*01:01:01:04, E*01:01:01:05, E*01:01:01:06, E*01:01:01:07, E*01:01:01:08, E*01:01:01:09, E*01:01:01:10, E*01:01:02, E*01:03:01:01, E*01:03:01:02, E*01:03:01:03, E*01:03:01:04, E*01:03:02:01, E*01:03:02:02, E*01:03:03, E*01:03:04, E*01:03:05, E*01:04, E*01:05, E*01:06, E*01:07, E*01:08N, E*01:09 and E*01:10. In some embodiments, the HLA-E polypeptide comprises an HLA-E*01:01 or HLA-E*01:03 allele.

In some embodiments, the exogenous antigenic polypeptide comprises a self-peptide. In some embodiments, the exogenous antigenic polypeptide comprises a tolerogenic polypeptide. In some embodiments, the exogenous antigenic polypeptide comprises an autoimmune disease antigen. In some embodiments, the exogenous antigenic polypeptide comprises the leader sequence of HSP60 (QMRPVSRVL) (SEQ ID NO: 17). In some embodiments, the exogenous antigenic polypeptide comprises a polypeptide listed in Table 1.

In some embodiments, the HLA-E polypeptide comprises one or more HLA-E a domains and a β2M polypeptide, or a fragment thereof.

In some embodiments, the HLA-E polypeptide is linked to a membrane anchor.

In some embodiments, the HLA-E polypeptide comprises a single chain fusion protein comprising an exogenous antigenic polypeptide linked to the HLA-E polypeptide via a linker. In some embodiments, the single chain fusion protein further comprises a membrane anchor. In some embodiments, the linker comprises a cleavable linker. In some embodiments, the membrane anchor comprises a glycophorin A (GPA) protein or a transmembrane domain thereof, or a small integral membrane protein 1 (SMIM1) or a transmembrane domain thereof.

In some embodiments, the exogenous antigenic polypeptide is bound to the exogenous antigen-presenting polypeptide covalently. In some embodiments, the exogenous antigenic polypeptide is bound to the exogenous antigen-presenting polypeptide non-covalently.

In some embodiments, the exogenous antigenic polypeptide is between about 8 amino acids in length to about 24 amino acids in length.

In some embodiments, the engineered enucleated erythroid cell further comprises an exogenous T regulatory costimulatory polypeptide on the cell surface. In some embodiments, the exogenous T regulatory costimulatory polypeptide is IL-17, IL-21, IL-18, IL-2, CD80, CD86, IL-15, TNFα, anti-DR3 agonist, 4-1BBL, or TGFβ.

In some embodiments, the engineered enucleated erythroid cell further comprises an exogenous costimulatory polypeptide on the cell surface. In some embodiments, the engineered enucleated erythroid cell further comprises an exogenous coinhibitory polypeptide on the cell surface.

In some embodiments, the engineered enucleated erythroid cell is a reticulocyte. In some embodiments, the engineered enucleated erythroid cell is an erythrocyte.

In another aspect, the disclosure provides a method or activating a T regulatory (Treg) cell, the method comprising contacting the Treg cell with an aAPC as described herein, thereby activating the Treg cell.

In another aspect, the disclosure provides a method of inhibiting an immune cell, the method comprising contacting the immune cell with an aAPC as described herein, thereby inhibiting the immune cell. In some embodiments, the immune cell is a cytotoxic CD8+ T cell or a natural killer cell.

In another aspect, the disclosure features a method of treating a subject in need of a modulated immune response, the method comprising contacting an immune cell of the subject with an aAPC as described herein, thereby treating the subject in need of a modulated immune response. In some embodiments, the immune cell is a T regulatory cell, a cytotoxic CD8+ T cell or a natural killer cell. In some embodiments, the contacting is performed in vitro or in vivo.

In one aspect, the present description provides a method of treating a subject having an autoimmune disease or inflammatory disease, the method comprising selecting an artificial antigen presenting cell (aAPC), wherein the aAPC is a engineered enucleated erythroid cell comprising an antigen-presenting polypeptide and at least one exogenous antigenic polypeptide that is specifically bound to the antigen-presenting polypeptide, wherein the antigen-presenting polypeptide comprises an HLA-E polypeptide, and administering the aAPC to the subject, thereby treating the subject having an autoimmune disease or inflammatory disease.

In some embodiments, the subject has an autoimmune disease. In some embodiments, the autoimmune disease is mediated by Tfh cells, Th1 cells, or Th17 cells. In some embodiments, the autoimmune disease is selected from the group consisting of type I diabetes, rheumatoid arthritis, graft versus host disease (GVHD), nephritis, multiple sclerosis, mixed connective tissue disorder, pemphigus vulgaris, bullous pemphigoid, membranous glomerulonephritis, neuromyelitis optica, autoimmune encephalomyelitis, autoimmune hepatitis, chronic inflammatory demyelinating polyradiculoneuropathy, dermatomyositis, giant cell arteritis, granulomatosis with polyangiitis, Kawasaki disease, lupus nephritis, polyarteritis nodosa, pyoderma gangrenosum, spondylarthritis, systemic lupus erythematosus, Takayasus arteritis.

In some embodiments, the exogenous antigenic polypeptide comprises the leader sequence of HSP60 (QMRPVSRVL) (SEQ ID NO: 17).

In some embodiments, the subject has an allergic disorder. In some embodiments, the allergic disorder is mediated by Th2 cells.

In some embodiments, the subject has an inflammatory disease. In some embodiments, the inflammatory disease is cardiac inflammatory disease, hepatic inflammatory disease, pancreatic inflammatory disease, inflammatory disease of the skin, and/or inflammatory disease of the gastrointestinal (GI) tract. For example, an inflammatory disease includes, but is not limited to, myocarditis, cardiomyopathy, endocarditis, pericarditis, cirrhosis, asthma (eosinophilic or non-eosinophilic), chronic obstructive pulmonary disease (COPD), asthma and COPD overlap syndrome (ACOS), atopic dermatitis, nasal polyps, an allergic response, chronic bronchitis, emphysema, hypersensitivity pneumonitis, allergic rhinitis, chronic rhino sinusitis with or without nasal polyps, inflammatory bowel disease, irritable bowel syndrome, ileitis, chronic inflammatory intestinal disease, fibrosis, eosinophilic esophagitis, vasculitis, urticaria, Churg Strauss syndrome, and inflammatory pain.

In another aspect, the disclosure features a method of expanding a population of regulatory T (Treg) cells, the method comprising: obtaining a population of cells from a subject, wherein the population comprises a Treg cell, contacting the population with an aAPC as described herein, wherein contacting the population with the aAPC induces proliferation of the Treg cell, thereby expanding the population of Treg cells. In some embodiments, the method further comprises isolating the Treg cell from the population of cells. In some embodiments, the method further comprises administering the Treg cell to the subject.

In another aspect, the disclosure features a method of making an aAPC described herein, the method comprising: introducing an exogenous nucleic acid encoding an exogenous antigen-presenting polypeptide described herein (e.g., an exogenous antigen-presenting polypeptide comprising an HLA-E polypeptide) into a nucleated erythroid precursor cell; and culturing the nucleated erythroid precursor cell under conditions suitable for enucleation and for production of the exogenous antigen-presenting polypeptide, thereby making the aAPC.

In another aspect, the disclosure features a method of making an aAPC as described herein, the method comprising: introducing an exogenous nucleic acid encoding an exogenous antigen-presenting polypeptide described herein into a nucleated erythroid precursor cell; introducing an exogenous nucleic acid encoding an exogenous antigenic polypeptide described herein into the erythroid precursor nucleated cell; and culturing the nucleated erythroid precursor cell under conditions suitable for enucleation and for production of the exogenous antigen-presenting polypeptide and the exogenous antigenic polypeptide, thereby making the aAPC.

In another aspect, the disclosure features a method of making an aAPC as described herein, the method comprising: introducing an exogenous nucleic acid encoding an exogenous antigen-presenting polypeptide described herein into a nucleated cell; culturing the nucleated erythroid precursor cell under conditions suitable for enucleation and production of the exogenous antigen-presenting polypeptide, thereby making an engineered enucleated erythroid cell; and contacting the engineered enucleated erythroid cell with at least one exogenous antigenic polypeptide, wherein the at least one exogenous antigenic polypeptide binds to the exogenous antigen-presenting polypeptide which is present on the cell surface of the engineered enucleated erythroid cell, thereby making the aAPC.

In some embodiments, the exogenous nucleic acid comprises DNA.

In some embodiments, the exogenous nucleic acid comprises RNA.

In some embodiments, the introducing step comprises viral transduction. In some embodiments, the introducing step comprises electroporation. In some embodiments, the introducing step comprises utilizing one or more of: liposome mediated transfer, adenovirus, adeno-associated virus, herpes virus, a retroviral based vector, lipofection, and a lentiviral vector.

In another aspect, the disclosure features an artificial antigen presenting cell (aAPC) comprising an engineered enucleated erythroid cell comprising an exogenous antigen-presenting polypeptide on the cell surface, wherein the exogenous antigen-presenting polypeptide comprises a human leukocyte antigen-G (HLA-G) polypeptide, and an exogenous antigenic polypeptide that is specifically bound to the exogenous antigen-presenting polypeptide. In some embodiments, the HLA-G polypeptide is selected from the group consisting of: HLA-G1, HLA-G2, HLA-G3, HLA-G4, HLA-G5, HLA-G6, and HLA-G7. In some embodiments, the HLA-G polypeptide is selected from the group consisting of: HLA-G1, HLA-G2, HLA-G5, and HLA-G6.

In some embodiments, an aAPC as described herein, wherein the exogenous antigenic polypeptide comprises the motif XI/LPXXXXXL (SEQ ID NO: 8).

In some embodiments, the HLA-G polypeptide comprises one or more HLA-G a domains and a β2M polypeptide, or a fragment thereof. In some embodiments, the HLA-G polypeptide is linked to a membrane anchor.

In some embodiments, the HLA-G polypeptide comprises a single chain fusion protein comprising an exogenous antigenic polypeptide linked to the HLA-G polypeptide via a linker. In some embodiments, the single chain fusion protein further comprises a membrane anchor.

In some embodiments, the linker comprises a cleavable linker.

In some embodiments, the membrane anchor comprises a glycophorin A (GPA) protein or a transmembrane domain thereof, or a small integral membrane protein 1 (SMIM1) or a transmembrane domain thereof.

In some embodiments, the exogenous antigenic polypeptide is bound to the exogenous antigen-presenting polypeptide covalently. In some embodiments, the exogenous antigenic polypeptide is bound to the exogenous antigen-presenting polypeptide non-covalently.

In some embodiments, the exogenous antigenic polypeptide is between about 8 amino acids in length to between about 24 amino acids in length.

In some embodiments, the aAPC is capable of suppressing a T cell that interacts with the aAPC. In some embodiments, the suppressing comprises inhibition of proliferation of a T cell, anergizing of a T cell, or induction of apoptosis of a T cell. In some embodiments, the T cell is a CD8+ T cell.

In some embodiments, the engineered enucleated erythroid cell further comprises at least one exogenous coinhibitory polypeptide. In some embodiments, the exogenous coinhibitory polypeptide is IL-10.

In some embodiments, the aAPC is capable of suppressing a B cell that interacts with the aAPC.

In some embodiments, the aAPC is capable of suppressing an NK cell that interacts with the aAPC.

In some embodiments, the aAPC is capable of suppressing a macrophage cell that interacts with the aAPC.

In some embodiments, the aAPC is capable of suppressing a dendritic cell that interacts with the aAPC.

In some embodiments, the engineered enucleated erythroid cell further comprises a checkpoint molecule on the cell surface. In some embodiments, the checkpoint molecule is selected from the group consisting of PD-L1, PD-L2, and OX40L.

In some embodiments, the engineered enucleated erythroid cell is a reticulocyte. In some embodiments, the engineered enucleated erythroid cell is an erythrocyte.

In one aspect, the present description provides a method of suppressing activity of an immune cell, the method comprising contacting the immune cell with an aAPC as described herein, thereby suppressing activity of the immune cell. In some embodiments, the immune cell is selected from the group consisting of a T cell, B cell, NK cell, macrophage, and dendritic cell. In some embodiments, the immune cell is a T cell. In some embodiments, the immune cell is a B cell. In some embodiments, the immune cell is an NK cell. In some embodiments, the immune cell is a macrophage. In some embodiments, the immune cell is a dendritic cell.

In another aspect, the disclosure features a method of treating a subject in need of a reduced immune response, the method comprising contacting an immune cell of the subject with an aAPC as described herein, thereby treating the subject in need of a reduced immune response.

In some embodiments, the subject has an autoimmune disease. In some embodiments, the autoimmune disease is selected from the group consisting of type I diabetes, rheumatoid arthritis, GVHD, nephritis and multiple sclerosis.

In some embodiments, the subject has an inflammatory disease.

In some embodiments, the subject has an allergic disease.

In some embodiments, the subject is in need of or has undergone a transplantation (e.g., an organ transplantation).

In another aspect, the disclosure features a method of making an aAPC as described herein, the method comprising: introducing an exogenous nucleic acid encoding the exogenous antigenic polypeptide into a nucleated erythroid precursor cell; and culturing the nucleated erythroid precursor cell under conditions suitable for enucleation and production of the exogenous antigenic polypeptide, thereby making an engineered enucleated erythroid cell, thereby making the aAPC.

In another aspect, the disclosure features a method of making an aAPC as described herein, the method comprising: introducing an exogenous nucleic acid encoding the exogenous antigen-presenting polypeptide into a nucleated erythroid precursor cell; introducing an exogenous nucleic acid encoding the exogenous antigenic polypeptide into the nucleated erythroid precursor cell; and culturing the nucleated erythroid precursor cell under conditions suitable for enucleation and production of both the exogenous antigen-presenting polypeptide and exogenous antigenic polypeptide, thereby making an engineered enucleated erythroid cell, thereby making the aAPC.

In another aspect, the disclosure features a method of making an aAPC as described herein, the method comprising: introducing an exogenous nucleic acid encoding the exogenous antigen-presenting polypeptide into a nucleated erythroid precursor cell; culturing the nucleated erythroid precursor cell under conditions suitable for enucleation and production of the exogenous antigen-presenting polypeptide, thereby making an engineered enucleated erythroid cell; and contacting the engineered enucleated erythroid cell with at least one exogenous antigenic polypeptide, wherein the at least one exogenous antigenic polypeptide binds to the exogenous antigen-presenting polypeptide which is present on the cell surface of the engineered enucleated erythroid cell, thereby making the aAPC.

In some embodiments, the exogenous nucleic acid comprises DNA. In some embodiments, the exogenous nucleic acid comprises RNA. In some embodiments, the introducing step comprises viral transduction. In some embodiments, the introducing step comprises electroporation. In some embodiments, the introducing step comprises utilizing one or more of: liposome mediated transfer, adenovirus, adeno-associated virus, herpes virus, a retroviral based vector, lipofection, and a lentiviral vector.

In some embodiments of any of the above aspects and embodiments, the erythroid cell is an enucleated erythroid cell.

### BRIEF DESCRIPTION OF THE DRAWINGS

The figures are meant to be illustrative of one or more features, aspects, or embodiments of the present disclosure and are not intended to be limiting.

FIG. 1 is a schematic showing an exemplary construct of an antigen-presenting polypeptide comprising an HLA-E polypeptide. In this construct, an exogenous antigenic polypeptide is linked to the β2M polypeptide, which is linked to one or more alpha domains of an HLA-E alpha chain (e.g., alpha1, alpha2, and alpha3 domains), which is linked to a membrane anchor, such as GPA or SMIM1.

FIGS. 2A-2C is a schematic showing exemplary constructs of antigen-presenting polypeptide comprising an HLA-G polypeptide. FIG. 2A depicts a construct which comprises an exogenous antigenic polypeptide linked to a β2M polypeptide, which is linked to one or more alpha domains of an HLA-G alpha chain (e.g., one or more of alpha1, alpha2, and alpha3 domains) linked to a membrane anchor, such as GPA or SMIM1. The construct further includes a β2M leader sequence. FIG. 2B depicts an open conformation (OC) construct (e.g., not fused to an exogenous antigenic polypeptide), which comprises a β2M polypeptide linked to one or more alpha domains of an HLA-G alpha chain (e.g., one or more of alpha1, alpha2, and alpha3 domains), which is linked to a membrane anchor, such as GPA or SMIM1, wherein the HLA-G open conformation is capable of binding an antigenic polypeptide. The construct further includes a β2M leader sequence. FIG. 2C depicts an antigen-presenting polypeptide comprising an HLA-G2 construct, which comprises HLA-G2 alpha1, and alpha2 domains linked to a membrane anchor, such as GPA or SMIM1. The construct further includes a β2m or alpha leader sequence.

### DETAILED DESCRIPTION

The present disclosure is based on the development of artificial antigen presenting cells (aAPCs) which comprise engineered erythroid cells (e.g., engineered enucleated erythroid cells) or enucleated cells (e.g., modified enucleated cells) that include, on their surface (e.g., on the plasma membrane), an HLA-E or HLA-G polypeptide. In one embodiment, the HLA-E or HLA-G polypeptide is bound to an exogenous antigenic polypeptide.

In particular, the present disclosure is based, at least in part, upon the surprising finding that engineered erythroid cells (e.g., engineered enucleated erythroid cells) or enucleated cells (e.g., modified enucleated cells) that include HLA-E or HLA-G can, inter alia, modulate specific immune cells.

In some embodiments, the aAPC cells as described herein include HLA-E and can stimulate or activate T regulatory cells, e.g., CD8+ T regulatory cells (Tregs). Activation of Treg cells results in the suppression or killing of other immune cells, including follicular helper T (Tfh), Th1 cells, and T helper 17 (Th17), which are T cells which are known to lead to autoreactivity. In some embodiments, the aAPC cells as described herein include HLA-E and can inhibit or suppress other immune cells, such as, for example, NK cells and cytotoxic CD8+ T cells.

In other embodiments, the aAPC cells described herein include HLA-G and can inhibit or suppress certain immune cell populations, e.g., natural killer (NK) cells, T cells, B cells, macrophages, dentritic cells (DC), etc.

In some embodiments, HLA-G and HLA-E are capable of inhibiting cytotoxic functions of NK cells and cytotoxic CD8+ T cells, inhibiting NK and T cell proliferaton, promoting generation of CD8 and CD4 regulatory T cells, and/or inhibiting DC maturation and antigen presentation.

Accordingly, the aAPC cells described herein comprise engineered erythroid cells (e.g., engineered enucleated erythroid cells) or enucleated cells (e.g., modified enucleated cells) that include HLA-E and/or HLA-G and can be used in the treatment of inflammatory diseases or autoimmune diseases.

In some embodiments, the HLA-E polypeptide is a single chain fusion polypeptide comprising an HLA-E polypeptide linked to an antigenic exogenous peptide, such as heat shock protein 60 (HSP60), a self antigen, or a portion thereof, e.g., the leader sequence of HSP60. In some embodiments, the HLA-E polypeptide is a single chain fusion polypeptide comprising or consisting of the ectodomain of an HLA-E polypeptide (e.g., alpha1, alpha2, and alpha3 domains), beta-2 microglobulin (β2M) polypeptide, and a membrane anchor (e.g., a GPA transmembrane domain), wherein the single chain fusion polypeptide is optionally linked to an antigenic exogenous polypeptide. It has been shown that an HLA-E fusion protein comprising HLA-E and the leader sequence of HSP60 promotes the activation of T regulatory cells resulting in protection from pathology in autoimmune diseases such as, but not limited to, type 1 diabetes, rheumatoid arthritis, multiple sclerosis, and nephritis (see, e.g., Jiang et al. 2010 J. Clin Invest. 120(10):3641-3650; Leavenworth et al. J Clin Invest. 2013; 123(3):1382-1389).

In other embodiments, the HLA-G polypeptide is a single chain fusion polypeptide comprising an HLA-G polypeptide linked to an exogenous polypeptide, e.g., an exogenous peptide having the motif XI/LPXXXXXL (SEQ ID NO: 8). In some embodiments, the HLA-G polypeptide is a single chain fusion polypeptide comprising or consisting of the ectodomain of an HLA-G polypeptide (e.g., alpha1, alpha2, and alpha3 domains of an HLA-G1 or an HLA-G5 isoform polypeptide; alpha1 and alpha3 domains of an HLA-G2 or an HLA-G6 isoform polypeptide; alpha1 and alpha 2 domains of an HLA-G4 isoform polypeptide; alpha1 and alpha2 domains of an HLA-G4 isoform polypeptide; or alpha1 domain of an HLA-G3 or an HLA-G7 polypeptide), β2M polypeptide, and a membrane anchor (e.g., a GPA transmembrane domain), wherein the single chain fusion polypeptide is optionally linked to an antigenic exogenous polypeptide. In some embodiments, the HLA-G polypeptide is a single chain fusion polypeptide comprising or consisting of one or more alpha domains of an HLA-G alpha chain (e.g., alpha1, alpha2, and/or alpha3 domains of an HLA-G1 or an HLA-G5 isoform polypeptide; alpha1 and alpha3 domains of an HLA-G2 or an HLA-G6 isoform polypeptide; alpha1 and alpha 2 domains of an HLA-G4 isoform polypeptide; alpha1 and alpha2 domains of an HLA-G4 isoform polypeptide; or alpha1 domain of an HLA-G3 or an HLA-G7 polypeptide), β2M polypeptide, and a membrane anchor (e.g., a GPA transmembrane domain), wherein the single chain fusion polypeptide is optionally linked to an antigenic exogenous polypeptide. In some embodiments, the HLA-G polypeptide is a single chain fusion polypeptide comprising or consisting of one or more alpha domains of an HLA-G alpha chain (e.g., alpha1, alpha2, and/or alpha3 domains of an HLA-G1 or an HLA-G5 isoform polypeptide; alpha1 and alpha3 domains of an HLA-G2 or an HLA-G6 isoform polypeptide; alpha1 and alpha 2 domains of an HLA-G4 isoform polypeptide; alpha1 and alpha2 domains of an HLA-G4 isoform polypeptide; or alpha1 domain of an HLA-G3 or an HLA-G7 polypeptide), and a membrane anchor (e.g., a GPA transmembrane domain), wherein the single chain fusion polypeptide is optionally linked to an antigenic exogenous polypeptide. In some embodiments, the HLA-G polypeptide is not linked to an exogenous polypeptide. HLA-G interacts with inhibitory receptors on various immune cells (natural killer (NK) cells, T cells, B cells, macrophages, dendritic cells, etc.). Three HLA-G receptors have been described: ILT2/CD85j/LILRB1 (ILT2), ILT4/CD85d/LILRB2 (ILT4), and KIR2DL4/CD158d (KIR2DL4). ILT2 is expressed by B cells, some T cells, some NK cells, and all monocytes/dendritic cells, but ILT4 is myeloid-specific and only expressed by monocytes/dendritic cells. KIR2DL4 is mainly restricted to the CD56bright subsets of NK cells. ILT2 and ILT4 are inhibitory receptors and KIR2DL4 is likely also capable of sending inhibitory signals as well. Through interaction with these receptors, HLA-G has been shown to inhibit cytotoxic functions of NK cells and cytotoxic CD8+ T cells, inhibit NK and T cell proliferation, promote generation of CD8+ and CD4+ regulatory T cells, and inhibit DC maturation and antigen presentation. In some embodiments, an exogenous antigen-presenting polypeptide described herein comprising an HLA-G polypeptide is capable of binding to one or more HLA-G receptors, such as ILT4, ILT2, and/or KIR2DL4 (e.g., present on the surface of a NK cell, a CD8+ T cell, a CD4+ T cell, a B cell, a monocyte, and/or a dendritic cell).

Thus, the aAPCs described herein can inhibit certain immune cell populations and suppress the activity of these immune cell populations, e.g., natural killer (NK) cells, T cells, B cells, macrophages, and dendritic cells. In another embodiments, the aAPCs described herein can activate or stimulate other immune cell populations, e.g., T regulatory cells.

Accordingly, the present disclosure relates to aAPCs, in particular which comprise engineered erythroid cells (e.g., engineered enucleated erythroid cells) or enucleated cells (e.g., modified enucleated cells) that include at least one exogenous antigen-presenting polypeptide which is an HLA-E or HLA-G polypeptide. In some embodiments, the aAPCs further include at least one exogenous antigenic polypeptide. In some embodiments, the exogenous antigenic polypeptide is a self-peptide, e.g., the leader sequence of the HSP60 peptide. In some embodiments, the aAPCs further include at least one Treg costimulatory polypeptide, such as IL-15, or an IL-15/IL-15Ra fusion polypeptide. In other embodiments, the aAPCs further include one or more costimulatory polypeptide. In other embodiments, the aAPCs further include one or more coinhibitory polypeptides.

In some embodiments, the aAPCs are engineered to modulate (e.g., activate) the activity of CD8+ regulatory T cells, and specific populations thereof, e.g., CD8+, CD122+, Ly49+ regulatory T cells. In other embodiments, the aAPCs are engineered to modulate (e.g., suppress) the activity of NK cells, T cells, e.g., cytotoxic CD8+ T cells, B cells, macrophages, and/or DCs.

In some embodiments of the present disclosure, the engineered erythroid cells are engineered enucleated erythroid cells, e.g., reticulocytes or erythrocytes. In some embodiments of the present disclosure, the enucleated cell (e.g., modified enucleated cell) is a reticulocyte, an erythrocyte or a platelet.

Many modifications and other embodiments of the aAPCs set forth herein will easily come to mind to one skilled in the art to which this disclosure pertains having the benefit of the teachings presented in the foregoing descriptions and the associated drawings. Therefore, it is to be understood that the disclosure herein is not to be limited to the specific embodiments disclosed and that modifications and other embodiments are intended to be included within the scope of the appended claims. Although specific terms are employed herein, they are used in a generic and descriptive sense only and not for purposes of limitation.

### Definitions

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows, e.g., component and do not exclude or preclude the presence of additional, non-recited components, features, element, members, steps, known in the art or disclosed therein.

As used herein, the terms “such as,” “for example,” and the like are intended to refer to exemplary embodiments and not to limit the scope of the present disclosure.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, preferred materials and methods are described herein.

As used herein, the term “codon-optimized” refers to the modification of codons in the gene or coding regions of a nucleic acid molecule to reflect the typical codon usage of the host organism (e.g., a human erythroid cell) without altering the polypeptide encoded by the nucleic acid molecule. Such optimization includes replacing at least one, or more than one, or a significant number, of codons with one or more codons that are more frequently used in the genes of the host organism. Codon optimization may improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, “dose” refers to a specific quantity of a pharmacologically active material for administration to a subject for a given time. Unless otherwise specified, the doses recited refer to a plurality of engineered erythroid cells (e.g., engineered enucleated erythroid cells) comprising an HLA-E or HLA-G polypeptide, as described herein. In some embodiments, a dose of engineered erythroid cells (e.g., engineered enucleated erythroid cells) refers to an effective amount of engineered erythroid cells. When referring to a dose for administration, in an embodiment of any one of the methods, compositions or kits provided herein, any one of the doses provided herein is the dose as it appears on a label/label dose.

As used herein, the term an “antigen-presenting cell (APC)” refers to a cell that can process and display foreign antigens in association with major histocompatibility complex (MHC) molecules on its surface.

As used herein, the term an “artificial antigen-presenting cell (aAPC)” refers to cells that have been engineered to introduce one or more HLA-E or HLA-G polypeptide. In some embodiments, the aAPC further comprises an exogenous antigenic polypeptide specifically bound to the HLA-E or HLA-G polypeptide.

As used herein, the term “inflammatory disease” refers generally to refers to a disease or disorder characterized by chronic or acute inflammation. In various embodiments, the inflammatory disease is a cardiac inflammatory disease, hepatic inflammatory disease, pancreatic inflammatory disease, inflammatory disease of the skin, and/or inflammatory disease of the gastrointestinal (GI) tract. For example, an inflammatory disease includes, but is not limited to, myocarditis, cardiomyopathy, endocarditis, pericarditis, cirrhosis, asthma (eosinophilic or non-eosinophilic), chronic obstructive pulmonary disease (COPD), asthma and COPD overlap syndrome (ACOS), atopic dermatitis, nasal polyps, an allergic response, chronic bronchitis, emphysema, hypersensitivity pneumonitis, allergic rhinitis, chronic rhinosinusitis with or without nasal polyps, inflammatory bowel disease, irritable bowel syndrome, ileitis, chronic inflammatory intestinal disease, fibrosis, eosinophilic esophagitis, vasculitis, urticaria, Churg Strauss syndrome, and inflammatory pain.

As used herein, the term “autoimmune disease” refers generally to diseases or conditions in which a subject's immune system attacks the body's own cells, causing tissue destruction or damage. In particular, in some embodiments, the term “autoimmune disease” includes any autoimmune disease that is modulated by Follicular helper T (Tth), Th1 cells, and/or T helper 17 (Th17) T cells (see, e.g., Zhang et al., J Immunol 2017, 198 (1 Supplement) 55.13; Jeon et al., Immune Netw. 2016, 16(4): 219-232; and Noack, et al., Autoimmunity Reviews, 13; 6, 2014: 668-677), the contents of which are incorporated by reference herein). For example, autoimmune diseases include, but are not limited to, rheumatoid arthritis (RA), juvenile idiopathic arthritis, rheumatoid spondylitis, ankylosing spondylitis, osteoarthritis, gouty arthritis, psoriatic arthritis, juvenile rheumatoid arthritis, type I diabetes (T1D), multiple sclerosis (MS), mixed connective tissue disorder, graft versus host disease (GVHD), autoimmune uveitis, nephritis, psoriasis, systemic lupus erythematosus (SLE), herpetic stromal keratitis (HSK), asthma, Crohn's disease, ulcerative colitis, spondylarthritis, active axial spondyloarthritis (active axSpA) and non-radiographic axial spondyloarthritis (nr-axSpA), pemphigus vulgaris, bullous pemphigoid, membranous glomerulonephritis, neuromyelitis optica, autoimmune encephalomyelitis, autoimmune hepatitis, chronic inflammatory demyelinating polyradiculoneuropathy, dermatomyositis, giant cell arteritis, granulomatosis with polyangiitis, Kawasaki disease, lupus nephritis, polyarteritis nodosa, pyoderma gangrenosum, and takayasu's arteritis.

Autoimmune diseases may be diagnosed using blood tests, cerebrospinal fluid analysis, electromyogram (measures muscle function), and magnetic resonance imaging of the brain, but antibody testing in the blood, for self-antibodies (or auto-antibodies) is particularly useful. Usually, IgG class antibodies are associated with autoimmune diseases.

As used herein, the term “biological sample” refers to any type of material of biological origin isolated from a subject, including, for example, DNA, RNA, lipids, carbohydrates, and protein. The term “biological sample” includes tissues, cells and biological fluids isolated from a subject. Biological samples include, e.g., but are not limited to, whole blood, plasma, serum, semen, saliva, tears, urine, fecal material, sweat, buccal, skin, cerebrospinal fluid, bone marrow, bile, hair, muscle biopsy, organ tissue or other material of biological origin known by those of ordinary skill in the art. Biological samples can be obtained from subjects for diagnosis or research or can be obtained from healthy subjects, as controls or for basic research.

As used herein, the term “click reaction” refers to a range of reactions used to covalently link a first and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic by-products that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used. In some embodiments, the click reaction is fast, specific, and high-yield.

As used herein, the term “click handle” refers to a chemical moiety that is capable of reacting with a second click handle in a click reaction to produce a click signature. In some embodiments, a click handle is comprised by a coupling reagent, and the coupling reagent may further comprise a substrate reactive moiety.

As used herein, the term “cytokine” refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Cytokines can act both locally and distantly from a site of release. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of other cytokines. Non limiting examples of cytokines include, e.g., IL-1α, IL-β, IL-2, IL-3, IL-4, IL-5, IL-6, IL-7, IL-8, IL-9, IL-10, IL-11, IL-12/IL-23 P40, IL-13, IL-15, IL-17, IL-18, IL-21, IL-23, TGF-β, IFN-γ, GM-CSF, Groα, MCP-1 and TNF-α. In some embodiments, the engineered erythroid cells (e.g., engineered enucleated erythroid cells) or enucleated cells (e.g., modified enucleated cells) described herein include one or more cytokines (e.g., IL-15). In some embodiments, the cytokine comprises a membrane anchor domain. In some embodiments, the cytokine is present on the surface of the cell.

As used herein, the term “endogenous” is meant to refer to a native form of compound (e.g., a small molecule) or process. For example, in some embodiments, the term “endogenous” refers to the native form of a nucleic acid or polypeptide in its natural location in the organism or in the genome of an organism.

As used herein, the term “an engineered cell” refers to a genetically-modified cell or progeny thereof.

As used herein, the term “enucleated cell” refers to a cell that lacks a nucleus (e.g., due to a differentiation process such as erythropoiesis). In some embodiments, an enucleated cell is incapable of expressing a polypeptide. In some embodiments, an enucleated cell is an erythrocyte, a reticulocyte, or a platelet.

As used herein, “engineered enucleated cell” refers to a cell that originated from a genetically-modified nucleated cell or progeny thereof, and lacks a nucleus (e.g., due to differentiation). In some embodiments, the engineered enucleated cell includes an exogenous polypeptide that was produced by the genetically-modified nucleated cell or progeny thereof (e.g., prior to enucleation) from which the engineered enucleated cell originated.

As used herein, “engineered erythroid cell” refers to a genetically-modified erythroid cell or progeny thereof. Engineered erythroid cells include engineered nucleated erythroid cells (e.g., genetically-modified erythroid precursor cells) and engineered enucleated erythroid cells (e.g., reticulocytes and erythrocytes that originated from a genetically modified erythroid precursor cell).

As used herein, “engineered enucleated erythroid cell” refers to an erythroid cell that originated from a genetically-modified nucleated erythroid cell or progeny thereof, and lacks a nucleus (e.g., due to differentiation). In some embodiments, an engineered enucleated erythroid cell comprises an erythrocyte or a reticulocyte that originated from a genetically-modified nucleated erythroid cell or progeny thereof. In some embodiments, the engineered enucleated erythroid cell did not originate from an immortalized nucleated erythroid cell or progeny thereof.

An “erythroid precursor cell”, as used herein, refers to a cell capable of differentiating into a reticulocyte or erythrocyte. Generally, erythroid precursor cells are nucleated. Erythroid precursor cells include a cord blood stem cell, a CD34⁺ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), an induced pluripotent stem cell (iPSC), a mesenchymal stem cell (MSC), a polychromatic normoblast, and an orthochromatic normoblast. In some embodiments, an erythroid precursor cell is an immortal or immortalized cell. For example, immortalized erythroblast cells can be generated by retroviral transduction of CD34⁺ hematopoietic progenitor cells to express Oct4, Sox2, Klf4, cMyc, and suppress TP53 (e.g., as described in Huang et al. (2014) Mol. Ther. 22(2): 451-63, the entire contents of which are incorporated by reference herein).

As used herein, the term “exogenous nucleic acid” refers to a nucleic acid (e.g., a gene) which is not native to a cell, but which is introduced into the cell or a progenitor of the cell. An exogenous nucleic acid may include a region or open reading frame (e.g., a gene) that is homologous to, or identical to, an endogenous nucleic acid native to the cell. In some embodiments, the exogenous nucleic acid comprises RNA. In some embodiments, the exogenous nucleic acid comprises DNA. In some embodiments, the exogenous nucleic acid is integrated into the genome of the cell. In some embodiments, the exogenous nucleic acid is processed by the cellular machinery to produce an exogenous polypeptide. In some embodiments, the exogenous nucleic acid is not retained by the cell or by a cell that is the progeny of the cell into which the exogenous nucleic acid was introduced.

As used herein, the term “exogenous polypeptide” refers to a polypeptide that is introduced into or onto a cell, or is caused to be expressed by the cell by introducing an exogenous nucleic acid encoding the exogenous polypeptide into the cell or into a progenitor of the cell. In some embodiments, an exogenous polypeptide is a polypeptide encoded by an exogenous nucleic acid that was introduced into the cell or a progenitor of the cell, which nucleic acid is optionally not retained by the cell. In some embodiments, an exogenous polypeptide is a polypeptide conjugated to the surface of the cell by chemical or enzymatic means.

As used herein, the term “express” or “expression” refers to processes by which a cell produces a polypeptide, including transcription and translation. The expression of a particular polypeptide in a cell may be increased using several different approaches, including, but not limited to, increasing the copy number of genes encoding the polypeptide, increasing the transcription of a gene, and increasing the translation of an mRNA encoding the polypeptide.

As used herein, the terms “first”, “second”, and “third”, etc., with respect to exogenous polypeptides or nucleic acids are used for convenience of distinguishing when there is more than one type of exogenous polypeptide or nucleic acid. Use of these terms is not intended to confer a specific order or orientation of the exogenous polypeptides or nucleic acid unless explicitly so stated.

As used herein, the term “gene” is used broadly to refer to any segment of nucleic acid associated with expression of a given RNA or protein. Thus, genes include regions encoding expressed RNAs (which typically include polypeptide coding sequences) and, often, the regulatory sequences required for their expression. Genes can be obtained from a variety of sources, including cloning from a source of interest or synthesizing from known or predicted sequence information, and may include sequences designed to have specifically desired parameters.

As used herein the term “nucleic acid molecule” refers to a single or double-stranded polymer of deoxyribonucleotide and/or ribonucleotide bases. It includes, but is not limited to, chromosomal DNA, plasmids, vectors, mRNA, tRNA, siRNA, etc. which may be recombinant and from which exogenous polypeptides may be expressed when the nucleic acid is introduced into a cell.

As used herein, the term “pharmaceutically acceptable carrier” includes any of the standard pharmaceutical excipients, carrier or stabilizer which are not toxic or deleterious to a mammal being exposed thereto at the dosage and/or concentration employed.

As used herein, the terms “polypeptide”, “peptide” and “protein” are used interchangeably herein to refer to a polymer of amino acid residues. The terms “polypeptide”, “peptide” and “protein” also are inclusive of modifications including, but not limited to, glycosylation, phosphorylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally.

As used herein, polypeptides referred to herein as “recombinant” refers to polypeptides which have been produced by recombinant DNA methodology, including those that are generated by procedures which rely upon a method of artificial recombination, such as the polymerase chain reaction (PCR) and/or cloning into a vector using restriction enzymes.

As used herein, the terms “subject”, “individual” and “patient” are used interchangeably herein and refer to any mammalian subject for whom diagnosis, treatment, or therapy is desired, particularly humans. The methods described herein are applicable to both human therapy and veterinary applications. In some embodiments, the subject is a mammal (e.g., a human subject).

As used herein, the terms “therapeutically effective amount” and “effective amount” are used interchangeably to refer to an amount of an active agent (e.g. an engineered erythroid cell or an enucleated cell described herein) that is sufficient to provide the intended benefit (e.g. prevention, prophylaxis, delay of onset of symptoms, or amelioration of symptoms of a condition, e.g., an autoimmune disease, an inflammatory disease, an infectious disease or an allergic disease. In prophylactic or preventative applications, an effective amount may be administered to a subject susceptible to, or otherwise at risk of developing a disease, disorder or condition (e.g., an autoimmune disease, an inflammatory disease, an infectious disease or an allergic disease) to eliminate or reduce the risk, lessen the severity, or delay the onset of the disease, disorder or condition, including a biochemical, histologic and/or behavioral symptoms of the disease, disorder or condition, its complications, and intermediate pathological phenotypes. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein, a therapeutically effective amount may be initially determined using preliminary in vitro studies and/or animal models. A therapeutically effective amount may also be determined from human clinical data. The applied dose may be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference.

As used herein, the terms “treat,” “treating,” and/or “treatment” include abrogating, substantially inhibiting, slowing or reversing the progression of a disorder, disease or condition (e.g., an autoimmune disease, an inflammatory disease, an infectious disease or an allergic disease), substantially ameliorating clinical symptoms of a disorder, disease or condition, or substantially preventing the appearance of clinical symptoms of a disorder, disease or condition, obtaining beneficial or desired clinical results. Treating further refers to accomplishing one or more of the following: (a) reducing the severity of the disorder, disease or condition (e.g., an autoimmune disease, an inflammatory disease, an infectious disease or an allergic disease); (b) limiting development of symptoms characteristic of the disorder, disease or condition(s) being treated; (c) limiting worsening of symptoms characteristic of the disorder, disease or condition(s) being treated; (d) limiting recurrence of the disorder, disease or condition(s) in subjects that have previously had the disorder, disease or condition(s); and (e) limiting recurrence of symptoms in subjects that were previously asymptomatic for the disorder, disease or condition(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “variant” of a polypeptide refers to a polypeptide having at least one amino acid residue difference as compared to a reference polypeptide, e.g., one or more substitutions, insertions, or deletions. In some embodiments, a variant has at least about 70%, 75%, 80%, 85%, 90%, 91%, 92%, 93%, 94% 95%, 96%, 97%, 98%, or 99% identity to that polypeptide. A variant may include a fragment (e.g., an enzymatically active fragment of a polypeptide (e.g., an enzyme). In some embodiments, a fragment may lack up to about 1, 2, 3, 4, 5, 10, 20, 30, 40, 50, or 100 amino acid residues on the N-terminus, C-terminus, or both ends (each independently) of a polypeptide, as compared to the full-length polypeptide. Variants may occur naturally or be non-naturally occurring. Non-naturally occurring variants may be generated using mutagenesis methods known in the art. Variant polypeptides may comprise conservative or non-conservative amino acid substitutions, deletions or additions.

As used herein, the term “sequence identity” or “identity,” in reference to nucleic acid and amino acid sequences refers to the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482; by means of the local homology algorithm of Neddleman and Wunsch, 1970, J. Mol. Biol. 48, 443; by means of the similarity search method of Pearson and Lipman, 1988, Proc. Natl. Acad. Sci. USA 85, 2444; or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The term “antigenic polypeptide” as used herein refers to an exogenous polypeptide that is capable of binding to an exogenous antigen-presenting polypeptide. In some embodiments, the antigenic polypeptide together with the exogenous antigen-presenting polypeptide are capable of inducing an immune response.

The term “tolerogenic polypeptide” as used herein, is any peptide that can promote immune tolerance. These tolerogenic effects include, but are not limited to, the regulation of T cells such as inducing T cell anergy, T cell apoptosis and induction of Tregs. Exemplary tolerogenic polypeptides are set forth in Table 1.

The term “exogenous antigen-presenting polypeptide” as used herein, refers to the cell surface proteins HLA-E and HLA-G that are capable of binding antigens and displaying them on the cell surface for recognition by the appropriate immune cells.

The term “HLA-E polypeptide,” “HLA-E molecule” or “HLA-E” (also known as HLA class I histocompatibility antigen, alpha chain E), refers to a non-classical MHC class I molecule comprising a heavy a chain comprising one or more of alpha1, alpha2, and alpha3 domains. The full length a heavy chain of HLA-E is approximately 45 kDa and its gene contains 8 exons. Exon one encodes the leader peptide, exons 2 and 3 encode the alpha1 and alpha2 domains, which both bind the peptide, exon 4 encodes the alpha3 domain, exon 5 encodes the transmembrane region, and exons 6 and 7 encode the cytoplasmic tail. As described herein, an HLA-E polypeptide can comprise less than all three of the endogenous alpha domains (i.e., the HLA-E polypeptide can comprise one, two or three of the alpha domains; also referred to herein as alpha heavy domains). In some embodiments, an HLA-E polypeptide comprises the ectodomain of a naturally-occurring HLA-E polypeptide (e.g., alpha1, alpha2, and alpha3 domains) and excludes the transmembrane domain and the cytoplasmic tail of the naturally-occurring HLA-E polypeptide. In some embodiments, an HLA-E polypeptide comprises the ectodomain of a naturally occurring HLA-E polypeptide (e.g., alpha1, alpha2, and alpha3 domains) and is fused to a membrane anchor (e.g., a glycophorin A (GPA) transmembrane domain). In some embodiments, an HLA-E polypeptide comprises the ectodomain of a naturally-occurring HLA-E polypeptide (e.g., alpha1, alpha2, and alpha3 domains), and is fused to a membrane anchor (e.g., a GPA transmembrane domain) which comprises an HLA-E cytoplasmic domain. As described herein, an HLA-E polypeptide also includes a heavy alpha chain that is bound or linked to a light chain (i.e., beta-2 microglobulin or β2M polypeptide), to form a heterodimer (e.g., as a single chain fusion polypeptide). In some embodiments, an HLA-E polypeptide binds or is bound to an exogenous antigenic polypeptide, and/or is linked or fused to a membrane anchor. In some embodiments, an HLA-E polypeptide comprises an “HLA-E single chain fusion polypeptide,” wherein the HLA-E polypeptide comprises one or more alpha domains of an HLA-E heavy chain (e.g., alpha1, alpha2, and alpha3 domains) linked to β2M polypeptide, and optionally the β2M polypeptide is linked to an exogenous antigenic polypeptide. In some embodiments, the single chain fusion polypeptide includes a membrane anchor.

HLA-E has the least polymorphisms of all the MHC class I genes, presenting only 11 alleles encoding three distinct proteins (International Immunogenetics Database, version 3.12.0). Overall, three allele groups (HLA-E*01:01, HLA-E*01:03, and HLA-E*01:04) have been described in diverse populations. However, only two allele groups (HLA-E*01:01 and HLA-E*01:03) are found in worldwide populations. An A/G variation at codon 107 defines HLA-E*01:01 and HLA-E*01:03 (see, e.g., the world wide web at hla.alleles.org/data/txt/e_nuc.txt), resulting in an arginine at position 107 in HLA-E*01:01 (HLA-E^{107R}) being replaced by a glycine in HLA-E*01:03 (HLA-E^{107G}) (see, e.g., Zheng et al., Cancer Sci. 2015 May; 106(5): 522-528). HLA-E peptide complexes on the cell surface can activate CD8 T regulatory cells. Regulatory T cells contribute to the suppression of other immune cells including Tfh, Th1 cells and Th17 cells, which are known to trigger autoimmunity (see Wieten et al. Tissue Antigens, 2014, 84, 523-535).

The term “HLA-G polypeptide,” “HLA-G molecule” or “HLA-G” (also known as HLA class I histocompatibility antigen, alpha chain G), refers to a non-classical MHC class I molecule comprising a heavy a chain comprising one of more of alpha1, alpha2, and alpha3 domains. The full length a heavy chain of HLA-G is approximately 45 kDa and its gene contains 8 exons. Exon one encodes the leader peptide, exons 2 and 3 encode the alpha1 and alpha2 domain, which both bind the peptide, exon 4 encodes the alpha3 domain, exon 5 encodes the transmembrane region, and exon 6 encodes the cytoplasmic tail. As described herein, an HLA-G polypeptide can comprise less than all three of the endogenous alpha domains (i.e., the HLA-G polypeptide can comprise one, two or three of the alpha domains; also referred to herein as alpha heavy domains). In some embodiments, an HLA-G polypeptide comprises the ectodomain of a naturally-occurring HLA-G polypeptide (e.g., one or more of alpha1, alpha2, and alpha 3 domains) and excludes the transmembrane domain and the cytoplasmic tail of the naturally-occurring HLA-G polypeptide. For example, in some embodiments, a HLA-G polypeptide comprises alpha1, alpha2 and alpha 3 domains of an HLA-G1 or an HLA-G5 isoform polypeptide. In some embodiments, a HLA-G polypeptide comprises alpha1 and alpha3 domains of a HLA-G2 or an HLA-G6 isoform polypeptide. In some embodiments, a HLA-G polypeptide comprises alpha1 and alpha 2 domains of an HLA-G4 isoform polypeptide. In some embodiments, a HLA-G2 polypeptide comprises alpha1 and alpha2 domains of an HLA-G4 isoform polypeptide. In some embodiments, a HLA-G polypeptide comprises an alpha1 domain of an HLA-G3 or an HLA-G7 polypeptide. In some embodiments, an HLA-G polypeptide comprises the ectodomain of a naturally occurring HLA-G polypeptide (e.g., one or more of alpha1, alpha2, and alpha3 domains) and is fused to a membrane anchor (e.g., a glycophorin A (GPA) transmembrane domain). In some embodiments, an HLA-G polypeptide comprises the ectodomain of a naturally-occurring HLA-G polypeptide (e.g., one or more of alpha1, alpha2, and alpha3 domains), and is fused to a membrane anchor (e.g., a GPA transmembrane domain) which comprises an HLA-G cytoplasmic domain. As described herein, an HLA-G polypeptide also includes an HLA-G heavy chain that is bound or linked to a light chain (i.e., beta-2 microglobulin or β2M polypeptide), to form a heterodimer (e.g., as a single chain fusion polypeptide). In some embodiments, an HLA-G polypeptide is not bound or linked to a light chain (i.e., a β2M polypeptide). In some embodiments, an HLA-G polypeptide binds or is bound to an exogenous antigenic polypeptide, and/or is linked to a membrane anchor. In some embodiments, an HLA-G polypeptide comprises an “HLA-G single chain fusion polypeptide,” wherein the HLA-G polypeptide comprises one or more alpha domains of an HLA-G heavy chain (e.g., one or more of alpha1, alpha2, and alpha3 domains) linked to β2M polypeptide, and optionally the β2M polypeptide is linked to an exogenous antigenic polypeptide. In some embodiments, the single chain fusion polypeptide includes a membrane anchor.

HLA-G is normally expressed on fetal derived placental cells. HLA-G comprises about 50 alleles encoding 16 different full length proteins, two truncated and 7 isoforms including HLA-G1, HLA-G2, HLA-G3, HLA-G4, HLA-G5, HLA-G6, and HLA-G7 (Carosella, Blood 2008, 11; 10, 4862). The membrane-bound HLA-G1 molecule and its soluble counterpart HLA-G5 have an identical extracellular structure, which is classic HLA class I-like: a heavy chain of 3 globular domains noncovalently bound to β2M polypeptide. The other isoforms are of simpler structure: lacking one or 2 globular domains, they are smaller, and should not bind β2M polypeptide or present peptides. In some embodiments, the HLA-G isoform included on the aAPCs as described herein is the HLA-G1 or HLA-G2 isoform. In some embodiments, the HLA-G isoform included on the aAPCs described herein is the soluble HLA-G5 or HLA-G6 isoform fused to GPA to anchor the molecule to the cell membrane. HLA-G multimers, e.g., dimers, may also be included on the aAPC cells described herein. The most common HLA-G allele is the HLA-G1*01:01:01:01 allele.

Exogenous antigen-presenting polypeptides HLA-E and HLA-G are described in more detail herein.

The term “costimulatory polypeptide” as used herein, refers to any polypeptide on an antigen presenting cell (e.g., an aAPC) that specifically binds to a cognate costimulatory molecule on an immune cell (e.g., an MHC molecule, B and T lymphocyte attenuator (CD272), and a Toll like receptor), thereby providing a signal which, in addition to the primary signal provided by binding of a an exogenous antigen-presenting polypeptide that includes an exogenous antigenic polypeptide, mediates an immune cell (e.g., T cell) response, including, but not limited to, proliferation, activation, differentiation, and the like, of the immune cell. A costimulatory polypeptide also encompasses, inter alia, an antibody that specifically binds with a costimulatory molecule present on a T cell. Exemplary exogenous costimulatory polypeptides are described in more detail below.

The term “exogenous coinhibitory polypeptide” as used herein refers to any polypeptide that suppresses an immune cell, including inhibition of immune cell activity, inhibition of immune cell proliferation, anergizing of an immune cell, or induction of apoptosis of an immune cell. Exemplary exogenous coinhibitory polypeptides are described in more detail below.

The term “Treg costimulatory polypeptide” as used herein refers to an exogenous polypeptide that expands regulatory T-cells (Tregs). In some embodiments, a Treg costimulatory polypeptide stimulates Treg cells by stimulating at least one of three signals involved in Treg cell development. Exemplary exogenous Treg costimulatory polypeptides are described in more detail below.

As used herein, the term “sufficient to stimulate an immune cell” refers to an amount or level of a signaling event or stimulus, e.g., of exogenous stimulatory polypeptide, that promotes a cellular response of an immune cell.

As used herein, the terms “activating immune cells” or “immune cell activation” refer to a process (e.g., a signaling event) causing or resulting in one or more cellular responses of an immune cell, selected from: proliferation, differentiation, cytokine secretion, cytotoxic effector molecule release, cytotoxic activity, and expression of activation markers. As used herein, an immune cell that has received an activating signal demonstrates one or more cellular responses, selected from proliferation, differentiation, cytokine secretion, cytotoxic effector molecule release, cytotoxic activity, and expression of activation markers. Suitable assays to measure immune cell activation are known in the art and are described herein.

As used herein, the terms “expanding an immune cell” or “immune cell expansion” refer to a process wherein an immune cell undergoes a series of cell divisions and thereby expands in cell number. Suitable assays to measure immune cell expansion are known in the art and are described herein.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that, in some embodiments, subsequently induces a signal transduction event, such as binding the TCR/CD3 complex. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. “Activation” includes activation of CD8+ T cells, activation of CD4+ T cells, stimulation of cytotoxic activity of T cells, stimulation of cytokine secretion by T cells, detectable effector functions, modification of the differentiation state of a T cell (e.g., promote expansion and differentiation from T effector to T memory cell), and/or any combination thereof. The term “activated T cells” refers to, among other things, T cells that are undergoing cell division.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the terms “suppressing immune cells” or “inhibiting immune cells” refer to a process (e.g., a signaling event) causing or resulting in the inhibition or suppression of one or more cellular responses or activities of an immune cell, selected from: proliferation, differentiation, cytokine secretion, cytotoxic effector molecule release, cytotoxic activity, and expression of activation markers, or resulting in anergizing of an immune cell or induction of apoptosis of an immune cell. Suitable assays to measure immune cell inhibition or suppression are known in the art and are described herein.

As used herein, “modulated immune response” refers to changing the form or character of the immune response, for example stimulation or inhibition of the immune response, e.g., as measured by ELISPOT assay (cellular immune response), ICS (intracellular cytokine staining assay) and major histocompatibility complex (MHC) tetramer assay to detect and quantify antigen-specific T cells, quantifying the blood population of antigen-specific CD4+ T cells, or quantifying the blood population of antigen specific CD8+ T cells by a measurable amount, or where the increase is by at least 10%, at least 20%, at least 30%, at least 40%, at least 50%, at least 60%, at least 70%, at least 80%, at least 90%, at least 95%, at least 96%, at least 97%, at least 98%, at least 99%, at least 100%, when compared to a suitable control.

### I. Cells of the Immune System

There are a large number of cellular interactions that comprise the immune system. These interactions occur through specific receptor-ligand pairs that signal in both directions so that each cell receives instructions based on the temporal and spatial distribution of those signals.

Cells of the immune system include lymphocytes, monocytes/macrophages, dendritic cells, the closely related Langerhans cells, natural killer (NK) cells, mast cells, basophils, and other members of the myeloid lineage of cells. In addition, a series of specialized epithelial and stromal cells provide the anatomic environment in which immunity occurs, often by secreting critical factors that regulate growth and/or gene activation in cells of the immune system, which also play direct roles in the induction and effector phases of the response. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

The cells of the immune system are found in peripheral organized tissues, such as the spleen, lymph nodes, Peyer's patches of the intestine and tonsils. Lymphocytes also are found in the central lymphoid organs, the thymus, and bone marrow where they undergo developmental steps that equip them to mediate the myriad responses of the mature immune system. A substantial portion of lymphocytes and macrophages comprise a recirculating pool of cells found in the blood and lymph, providing the means to deliver immunocompetent cells to sites where they are needed and to allow immunity that is generated locally to become generalized. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens through recombination of their genetic material (e.g. to create a T cell receptor and a B cell receptor). This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

Two broad classes of lymphocytes are recognized: the T-lymphocytes (T-cells), which are precursors of antibody-secreting cells, and B-lymphocytes (B-cells).

### Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter (Scwartz, R. H., “T cell anergy”, Annu. Rev. Immunol., Vol. 21: 305-334 (2003)) contribute to the downregulation of the immune response. A third mechanism is provided by active suppression of activated T cells by suppressor or regulatory CD4⁺ T (Treg) cells (Reviewed in Kronenberg, M. et al., “Regulation of immunity by self-reactive T cells”, Nature, Vol. 435: 598-604 (2005)). CD4⁺ Tregs that constitutively express the IL-2 receptor alpha (IL-2Rα) chain (CD4⁺ CD25⁺) are a naturally occurring T cell subset that are anergic and suppressive (Taams, L. S. et al., “Human anergic/suppressive CD4⁺ CD25⁺ T cells: a highly differentiated and apoptosis-prone population”, Eur. J. Immunol. Vol. 31: 1122-1131 (2001)). Depletion of CD4⁺CD25⁺ Tregs results in systemic autoimmune disease in mice. Furthermore, transfer of these Tregs prevents development of autoimmune disease. Human CD4⁺CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development (Battaglia, M. et al., “Rapamycin promotes expansion of functional CD4⁺CD25⁺Foxp3⁺ regulator T cells of both healthy subjects and type 1 diabetic patients”, J. Immunol., Vol. 177: 8338-8347, (2006)).

### T-Lymphocytes

T-lymphocytes derived from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. While antibody and B-cell receptors evolved to bind to and to protect against microorganisms in extracellular fluids, T cells recognize antigens on the surface of other cells and mediate their functions by interacting with, and altering, the behavior of these antigen-presenting cells (APCs). There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an APC that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the APC for long enough to become activated (“Chapter 24: The adaptive immune system,” Molecular Biology of the Cell, Alberts, B. et al., Garland Science, NY, (2002)).

T-cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4⁺ T cells); and those that express CD8 (CD8⁺ T cells). These cells differ in how they recognize antigen and in their effector and regulatory functions.

CD4⁺ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells, particularly CD8⁺ T cells, can develop into cytotoxic T-lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)).

T cell receptors (TCRs) recognize a complex consisting of a peptide derived by proteolysis of the antigen bound to a specialized groove of a class II or class I MHC protein. CD4⁺ T cells recognize only peptide/class II complexes while CD8⁺ T cells recognize peptide/class I complexes (Paul, W. E., “Chapter 1: The immune system: an introduction,” Fundamental Immunology, 4th Edition, Ed. Paul, W. E., Lippicott-Raven Publishers, Philadelphia, (1999)).

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4⁺ T cells with TCRs capable of recognizing the expressed cell surface complex. Thus, CD4⁺ T cells are specialized to react with antigens derived from extracellular sources (Paul, W. E., “Chapter 1: The immune system: an introduction,” Fundamental Immunology, 4th Edition, Ed. Paul, W. E., Lippicott-Raven Publishers, Philadelphia, (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8⁺ T cells expressing appropriate receptors. This gives the T cell system, particularly CD8⁺ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted (Paul, W. E., “Chapter 1: The immune system: an introduction,” Fundamental Immunology, 4th Edition, Ed. Paul, W. E., Lippicott-Raven Publishers, Philadelphia, (1999)).

T cells can also be classified based on their function as helper T cells; T cells involved in inducing cellular immunity; suppressor T cells; and cytotoxic T cells.

### Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes, and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4th Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching of the Ig class being expressed, either depend or are enhanced by the actions of T cell-derived cytokines (Paul, W. E., “Chapter 1: The immune system: an introduction,” Fundamental Immunology, 4th Edition, Ed. Paul, W. E., Lippicott-Raven Publishers, Philadelphia, (1999)).

CD4⁺ T cells tend to differentiate into cells that principally secrete the cytokines IL-4, IL-5, IL-6, and IL-10 (T_{H}2 cells) or into cells that mainly produce IL-2, IFN-γ, and lymphotoxin (T_{H}1 cells). The T_{H}2 cells are very effective in helping B-cells develop into antibody-producing cells, whereas the T_{H}1 cells are effective inducers of cellular immune responses, involving enhancement of microbicidal activity of monocytes and macrophages, and consequent increased efficiency in lysing microorganisms in intracellular vesicular compartments. Although CD4⁺ T cells with the phenotype of T_{H}2 cells (i.e., IL-4, IL-5, IL-6 and IL-10) are efficient helper cells, T_{H}1 cells also have the capacity to be helpers (Paul, W. E., “Chapter 1: The immune system: an introduction, “Fundamental Immunology, 4th Edition, Ed. Paul, W. E., Lippicott-Raven Publishers, Philadelphia, (1999)).

### T Cell Involvement in Cellular Immunity Induction

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. T_{H1} cells are effective in enhancing the microbicidal action, because they produce IFN-γ. In contrast, two of the major cytokines produced by T_{H2} cells, IL-4 and IL-10, block these activities (Paul, W. E., “Chapter 1: The immune system: an introduction,” Fundamental Immunology, 4th Edition, Ed. Paul, W. E., Lippicott-Raven Publishers, Philadelphia, (1999)).

### Cytotoxic T Lymphocytes

CD8⁺ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

### Lymphocyte Activation

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cyl, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the APC.

### T-Memory Cells

Following the recognition and eradication of pathogens through adaptive immune responses, the vast majority (90-95%) of T cells undergo apoptosis with the remaining cells forming a pool of memory T cells, designated central memory T cells (TCM), effector memory T cells (TEM), and resident memory T cells (TRM) (Clark, R. A., “Resident memory T cells in human health and disease”, Sci. Transl. Med., 7, 269rv1, (2015)). CD45RA is expressed on naïve T cells, as well as the effector cells in both CD4 and CD8. After antigen experience, central and effector memory T cells gain expression of CD45RO and lose expression of CD45RA. Thus either CD45RA or CD45RO is used to generally differentiate the naïve from memory populations. CCR7 and CD62L are two other markers that can be used to distinguish central and effector memory T cells. Naïve and central memory cells express CCR7 and CD62L in order to migrate to secondary lymphoid organs. Thus, naïve T cells are CD45RA+CD45RO−CCR7+CD62L+, central memory T cells are CD45RA−CD45RO+CCR7+CD62L+, and effector memory T cells are CD45RA−CD45RO+CCR7−CD62L−.

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases (Clark, R. A., “Resident memory T cells in human health and disease”, Sci. Transl. Med., Vol. 7, 269rv1, (2015)).

### B-Lymphocytes

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses (Paul, W. E., “Chapter 1: The immune system: an introduction,” Fundamental Immunology, 4th Edition, Ed. Paul, W. E., Lippicott-Raven Publishers, Philadelphia, (1999)).

Cross-linkage dependent B-cell activation requires that the antigen express multiple copies of the epitope complementary to the binding site of the cell surface receptors, because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes (Paul, W. E., “Chapter 1: The immune system: an introduction”, Fundamental Immunology, 4th Edition, Ed. Paul, W. E., Lippicott-Raven Publishers, Philadelphia, (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4⁺ T-cells. The CD4⁺ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell (Paul, W. E., “Chapter 1: The immune system: an introduction, “Fundamental Immunology, 4th Edition, Ed. Paul, W. E., Lippicott-Raven Publishers, Philadelphia, (1999)).

During cognate help for antibody production, the CD40 ligand is transiently expressed on activated CD4⁺ T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in pathogenic autoantibody production in human SLE patients (Desai-Mehta, A. et al., “Hyperexpression of CD40 ligand by B and T cells in human lupus and its role in pathogenic autoantibody production,” J. Clin. Invest. Vol. 97(9), 2063-2073, (1996)).

II. Artificial Antigen Presenting Cells (aPCs)

The present disclosure features aAPCs comprising engineered erythroid cells (e.g., engineered enucleated erythroid cells) or enucleated cells (e.g., modified enucleated cells) that are engineered to include an exogenous antigen presenting polypeptide comprising a HLA-E polypeptide and/or an exogenous antigen presenting polypeptide comprising a HLA-G polypeptide, and to activate or suppress specific populations of immune cells. In some embodiments, the cells are engineered to include an exogenous antigen presenting polypeptide comprising a HLA-E polypeptide, and activate T regulatory cells. In some embodiments, the cells are engineered to include an exogenous antigen presenting polypeptide comprising a HLA-E polypeptide, and suppress certain immune cells, e.g., NK cells or cytotoxic CD8+ T cells. In some embodiments, the cells are engineered to include an exogenous antigen presenting polypeptide comprising a HLA-G polypeptide, and suppress certain immune cells, e.g., natural killer (NK) cells, T cells, B cells, macrophages, and/or dentritic cells (DC).

In one aspect, the disclosure provides engineered erythroid cells (e.g., engineered enucleated erythroid cells) or enucleated cells (e.g., modified enucleated cells) that include an antigen-presenting polypeptide comprising either a HLA-E polypeptide or a HLA-G polypeptide.

The skilled artisan would appreciate, based upon the disclosure provided herein, that numerous immunoregulatory molecules can be used to produce aAPCs once armed with the teachings provided herein. That is, there is extensive knowledge in the art regarding the events and molecules involved in activation and induction of immune cells, e.g., activation of T regulatory cells, or inhibition of suppression of immune cells, e.g., natural killer (NK) cells, T cells, B cells, macrophages, and/or dentritic cells (DC).

In some aspects, the present disclosure provides an aAPC comprising an engineered erythroid cell (e.g., engineered enucleated erythroid cell) or an enucleated cell (e.g., a modified enucleated cell) comprising one or more exogenous polypeptide(s). Exogenous polypeptides of the present disclosure include, but are not limited to, exogenous antigenic polypeptides, exogenous antigen-presenting polypeptides including HLA-E polypeptides or HLA-G polypeptides, exogenous costimulatory polypeptides, exogenous coinhibitory polypeptides, cytokines, and exogenous Treg costimulatory polypeptides.

### Exogenous Antigenic Polypeptides

In certain embodiments, the aAPCs described herein include an antigen-presenting polypeptide (e.g., comprising an HLA-E polypeptide or an HLA-G polypeptide) bound (e.g., specifically bound) to an exogenous antigenic polypeptide. In some embodiments, the antigen-presenting polypeptide comprises an HLA-E polypeptide, and the exogenous antigenic polypeptide is selected from or comprises an amino acid sequence provided in Table 1, or a fragment or variant thereof. In some embodiments, the exogenous antigenic polypeptide is a tolerogenic polypeptide. In some embodiments, the exogenous antigenic polypeptide is derived from HSP60 (e.g., a leader sequence of HSP60).

TABLE 1
Exogenous Antigenic Polypeptides

| | | | | SEQ |
|---|---|---|---|---|
| | | | | ID |
| HLA Allele(s) | Peptide | Length | Origin | NO |
| HLA-E | SQQPYLQLQ | 9 | Gliadin | 11 |
| HLA-E | ALALVRMLI | 9 | ATP-binding | 12 |
| | | | cassette transporter | |
| | | | multidrug | |
| | | | resistance-associated | |
| | | | protein 7 | |
| HLA-E | YLLPRRGPRL | 10 | HCV core | 13 |
| HLA-E | AISPRTLNA | 9 | p24 protein of HIV- | 14 |
| | | | 1 | |
| HLA-E*01:03 | VMAPRTLIL | 9 | (HCMV)UL40 or | 15 |
| | | | HLA-Cw3 | |
| HLA-E*01:03 | SQAPLPCVL | 9 | Epstein-Barr virus | 16 |
| | | | (EBV)BZLF-1 | |
| HLA-E*01:01; | QMRPVSRVL | 9 | leader sequence of | 17 |
| HLA-E*01:03 | | | human HSP60 | |
| HLA-E*01:03 | HAVSEGTKAVTKYTSSK | 17 | Histone H2B type | 18 |
| | | | 1-L | |
| HLA-E*01:03 | PAETATPAPVEKSPAKK | 17 | Histone H1.5 | 19 |
| HLA-E*01:03 | AYVRLAPDYDALDVANK | 17 | 60S ribosomal | 20 |
| | | | protein L23a | |
| HLA-E*01:03 | HAVSEGTKAVTKYTSAK | 17 | Histone H2B type | 21 |
| | | | 1-J | |
| HLA-E*01:03 | AVSDGVIKVFNDMKVRK | 17 | Cofilin-1 | 22 |
| HLA-E*01:03 | HAVSEGTKAVTKYTSS | 16 | Histone H2B type | 23 |
| | | | 1-L | |
| HLA-E*01:03 | QLLQANPILEAFGNAK | 16 | Myosin-9 | 24 |
| HLA-E*01:03 | KSADTLWDIQKDLKDL | 16 | L-lactate | 25 |
| | | | dehydrogenase B | |
| | | | chain | |
| HLA-E*01:03 | AYVRLAPDYDALDVAN | 16 | 60S ribosomal | 26 |
| | | | protein L23a | |
| HLA-E*01:03 | HAVSEGTKAVTKYTSA | 16 | Histone H2B type | 27 |
| | | | 1-J | |
| HLA-E*01:03 | TGLIKGSGTAEVELKK | 16 | Pyruvate kinase | 28 |
| | | | isozymes M1/M2 | |
| HLA-3*01:03 | VSDGVIKVFNDMKVRK | 16 | Cofilin-1 | 29 |
| HLA-E*01:03 | ASGNYATVISHNPETK | 16 | 60S ribosomal | 30 |
| | | | protein L8 | |
| HLA-E*01:03 | TAEILELAGNAARDNK | 16 | Histone H2A type | 31 |
| | | | 1-D | |
| HLA-E*01:03 | HAVSEGTKAVTKYTSA | 16 | Histone H2B type | 32 |
| | | | 1-J | |
| HLA-E*01:03 | PAPVEKSPAKKKATK | 15 | Histone H1.5 | 33 |
| HLA-E*01:03 | SADTLWDIQKDLKDL | 15 | L-lactate | 34 |
| | | | dehydrogenase B | |
| | | | chain | |
| HLA-E*01:03 | TGLIKGSGTAEVELK | 15 | Pyruvate kinase | 35 |
| | | | isozymes M1/M2 | |
| HLA-E*01:03 | KSADTLWGIQKELQF | 15 | L-lactate | 36 |
| | | | dehydrogenase A | |
| | | | chain | |
| HLA-E*01:03 | HGSYEDAVHSGALND | 15 | T-complex protein 1 | 37 |
| | | | subunit alpha | |
| HLA-E*01:03 | SDGVIKVFNDMKVRK | 15 | Cofilin-1 | 38 |
| HLA-E*01:03 | AGNLGGGVVTIERSK | 15 | 60S ribosomal | 39 |
| | | | protein L22 | |
| HLA-E*01:03 | AQAAAPASVPAQAPK | 15 | 60S ribosomal | 40 |
| | | | protein L29 | |
| HLA-E*01:03 | PRKIEEIKDFLLTAR | 15 | 60S ribosomal | 41 |
| | | | protein L38 | |
| HLA-E*01:03 | SEGTKAVTKYTSSK | 14 | Histone H2B type | 42 |
| | | | 1-L | |
| HLA-E*01:03 | VLKQVHPDTGISSK | 14 | Histone H2B type | 43 |
| | | | 1-L | |
| HLA-E*01:03 | SWTAADTAAQITQR | 14 | HLA class I | 44 |
| | | | histocompatibility | |
| | | | antigen, Cw-1 alpha | |
| | | | chain | |
| HLA-E*01:03 | FISVGYVDDTQFVR | 14 | HLA class I | 45 |
| | | | histocompatibility | |
| | | | antigen, Cw-1 alpha | |
| | | | chain | |
| HLA-E*01:03 | NIDDGTSDRPYSHA | 14 | 60S ribosomal | 46 |
| | | | protein L27 | |
| HLA-E*01:03 | VLKQVHPDTGISSK | 14 | Histone H2B type | 47 |
| | | | 1-J | |
| HLA-E*01:03 | RKTVTAMDVVYALK | 14 | Histone H4 | 48 |
| HLA-E*01:03 | SADTLWGIQKELQF | 14 | L-lactate | 49 |
| | | | dehydrogenase A | |
| | | | chain | |
| HLA-E*01:03 | ASAETVDPASLWEY | 14 | Fascin | 50 |
| HLA-E*01:03 | TVVNKDVFRDPAL | 13 | 60S ribosomal | 51 |
| | | | protein L27 | |
| HLA-E*01:03 | KTVTAMDVVYALK | 13 | Histone H4 | 52 |
| HLA-E*01:03 | EGIPALDNFLDKL | 13 | Elongation factor 2 | 53 |
| HLA-E*01:03 | RVTIMPKDIQLAR | 13 | Histone H3.3C | 54 |
| HLA-E*01:03 | PVAVMAESAFSFK | 13 | COP9 signalosome | 55 |
| | | | complex subunit 8 | |
| HLA-E*01:03 | QTVAVGVIKAVDK | 13 | Elongation factor 1- | 56 |
| | | | alpha 1 | |
| HLA-E*01:03 | ILELAGNAARDNK | 13 | Histone H2A type | 57 |
| | | | 1-D | |
| HLA-E*01:03 | GTGASGSFKLNK | 12 | Histone H1.5 | 58 |
| HLA-E*01:03 | KQVHPDTGISSK | 12 | Histone H2B type | 59 |
| | | | 1-J | |
| HLA-E*01:03 | VGGTSDVEVNEK | 12 | 60 kDa heat shock | 60 |
| | | | protein, | |
| | | | mitochondrial | |
| HLA-E*01:03 | NSVVEASEAAYK | 12 | 14-3-3 protein eta | 61 |
| HLA-E*01:03 | ALRYPMAVGLNK | 12 | 60S ribosomal | 62 |
| | | | protein L36 | |
| HLA-E*01:03 | SLVSKGTLVQTK | 12 | Histone H1.5 | 63 |
| HLA-E*01:03 | PELAKSAPAPK | 11 | Histone H2B type | 64 |
| | | | 1-L | |
| HLA-E*01:03 | SEMEVQDAELK | 11 | Proliferation- | 65 |
| | | | associated protein | |
| | | | 2G4 | |
| HLA-E*01:03 | QTYSTEPNNLK | 11 | 60S ribosomal | 66 |
| | | | protein L28 | |
| HLA-E*01:03 | PMFIVNTNVPR | 11 | Macrophage | 67 |
| | | | migration inhibitory | |
| | | | factor | |
| HLA-E*01:03 | AGFAGDDAPR | 10 | Actin, cytoplasmic 1 | 68 |
| HLA-E*01:03 | RVNAGTLAVL | 10 | von Willebrand | 69 |
| | | | factor A domian- | |
| | | | containing protein 8 | |
| HLA-E*01:03 | IGQSKVFFR | 9 | Myosin-9 | 70 |
| HLA-E*01:03 | TAEILELAGNAARDNK | 16 | Histone H2A type | 71 |
| | | | 1-D | |
| HLA-E*01:03 | ILELAGNAARDNK | 13 | Histone H2A type | 72 |
| | | | 1-D | |
| HLA-E*01:03 | ALAGCHLEDTQRKLQKG | 17 | Polyamine- | 73 |
| | | | modulated factor 1- | |
| | | | binding protein 1 | |
| HLA-E*01:03 | MQLITRGKGAGTPNLI | 16 | Isthmin-1 | 74 |
| HLA-E*01:03 | KMKLRNTVHLSYLTV | 15 | Taste receptor type | 75 |
| | | | 2 member 50 | |
| HLA-E*01:03 | CRASQTISSYLDWYQ | 15 | Ig kappa chain V-I | 76 |
| | | | region OU | |
| HLA-E*01:03 | PAALTNKGNTVFA | 13 | Intraflagellar | 77 |
| | | | transport protein 88 | |
| | | | homologue | |
| HLA-E*01:03 | WTPGPSAGVTGIA | 13 | Mucin-19 | 78 |
| HLA-E*01:03 | ILRTIGKEAF | 10 | Trafficking protein | 79 |
| | | | particle complex | |
| | | | subunit 8 | |
| HLA-E*01:03 | RSCGYACTA | 9 | Isthmin-1 | 80 |
| HLA-E*01:03 | FPNGFSFIH | 9 | Sushi, von | 81 |
| | | | Willebrand factor | |
| | | | type A, EGF, and | |
| | | | pentraxin domain- | |
| | | | containing protein 1 | |
| HLA-E*01:03 | SHGPYIKLI | 9 | Major facilitator | 82 |
| | | | superfamily | |
| | | | domain-containing | |
| | | | protein 2A | |
| HLA-E*01:03 | AQAAAPASVPAQAPK | 15 | 60S ribosomal | 83 |
| | | | protein L29 | |
| HLA-E*01:03 | AYVRLAPDYDALDVANK | 17 | 60S ribosomal | 84 |
| | | | protein L23a | |
| HLA-E*01:03 | AYVRLAPDYDALDVAN | 16 | 60S ribosomal | 85 |
| | | | protein L23a | |
| HLA-E*01:03 | ASGNYATVISHNPETK | 16 | 60S ribosomal | 86 |
| | | | protein L8 | |
| HLA-E*01:03 | AGNLGGGVVTIERSK | 15 | 60S ribosomal | 87 |
| | | | protein L22 | |
| HLA-E*01:03 | PRKIEEIKDFLLTAR | 15 | 60S ribosomal | 88 |
| | | | protein L38 | |
| HLA-E*01:03 | NIDDGTSDRPYSHA | 14 | 60S ribosomal | 89 |
| | | | protein L27 | |
| HLA-E*01:03 | TVVNKDVFRDPAL | 13 | 60S ribosomal | 90 |
| | | | protein L27 | |
| HLA-E*01:03 | ALRYPMAVGLNK | 12 | 60S ribosomal | 91 |
| | | | protein L36 | |
| HLA-E*01:03 | QTYSTEPNNLK | 11 | 60S ribosomal | 92 |
| | | | protein L28 | |
| HLA-E*01:03 | PELAKSAPAPK | 11 | Histone H2B type | 93 |
| | | | 1-L | |
| HLA-E*01:03 | KQVHPDTGISSK | 12 | Histone H2B type | 94 |
| | | | 1-J | |
| HLA-E*01:03 | VLKQVHPDTGISSK | 14 | Histone H2B type | 95 |
| | | | 1-J | |
| HLA-E*01:03 | SEGTKAVTKYTSSK | 14 | Histone H2B type | 96 |
| | | | 1-L | |
| HLA-E*01:03 | HAVSEGTKAVTKYTSA | 16 | Histone H2B type | 97 |
| | | | 1-J | |
| HLA-E*01:03 | HAVSEGTKAVTKYTSS | 16 | Histone H2B type | 98 |
| | | | 1-L | |
| HLA-E*01:03 | HAVSEGTKAVTKYTSAK | 17 | Histone H2B type | 99 |
| | | | 1-J | |
| HLA-E*01:03 | HAVSEGTKAVTKYTSSK | 17 | Histone H2B type | 100 |
| | | | 1-L | |
| HLA-E*01:03 | HAVSEGTKAVTKYTSA | 16 | Histone H2B type | 101 |
| | | | 1-J | |
| HLA-E*01:03 | AGFAGDDAPR | 10 | Actin, cytoplasmic 1 | 102 |
| HLA-E*01:01 | SQAPLPCVL | 9 | Epstein-Barr virus | 103 |
| | | | (EBV)BZLF-1 | |
| HLA-E*01:01 | VMAPRTLFL | 9 | HLA-G leader | 104 |
| | | | peptide | |
| HLA-E*01:01 | QMRPVSRVL | 9 | leader sequence of | 17 |
| | | | human HSP60 | |
| HLA-E*01:01 | PKKTESHHKAKGK | 13 | Histone H2A type 3 | 105 |
| HLA-E*01:01 | AAVLEYL | 7 | Histone H2A type | 106 |
| | | | 2-B | |
| HLA-E*01:01 | AQAAAPASVPAQAPK | 25 | 60S ribosomal | 107 |
| | RTQAPTKASE | | protein L29 | |
| HLA-E*01:01 | KLEKEEEEGISQESSEEEQ | 19 | High mobility group pro- | 108 |
| | | | tein HMG-I/HMG-Y | |
| HLA-E*01:01 | GDRSEDFGVNEDLADSDAR | 19 | Annexin A1 | 109 |
| HLA-E*01:01 | VAPEEHPVLLTEAPLNPK | 18 | Actin, cytoplasmic 1 | 110 |
| HLA-E*01:01 | STAGDTHLGGEDFDNR | 16 | Heat shock cognate | 111 |
| | | | 71 kDa protein | |
| HLA-E*01:01 | KVPQVSTPTLVEVSR | 15 | Serum albumin | 112 |
| HLA-E*01:01 | PDPAKSAPAPKKGSK | 15 | Histone H2B type | 113 |
| | | | 1-H | |
| HLA-E*01:01 | LQAEIEGLKGQR | 12 | Keratin, type II | 114 |
| | | | cytoskeletal 8 | |
| HLA-E*01:01 | PDPAKSAPAPK | 11 | Histone H2B type | 115 |
| | | | 1-H | |
| HLA-E*01:01 | PELAKSAPAPK | 11 | Histone H2B type | 116 |
| | | | 1-L | |
| HLA-E*01:01 | PEPVKSAPVPK | 11 | Histone H2B type | 117 |
| | | | 1-M | |
| HLA-E*01:01 | AAPATRAAL | 9 | Solute carrier family | 118 |
| | | | 15 member 4 | |
| HLA-E*01:01 | SAPSRATAL | 9 | BTB/POZ domain- | 119 |
| | | | containing protein | |
| | | | KCTD18 | |
| HLA-E*01:01 | ILNFPPPP | 8 | Caprin-2 | 120 |
| HLA-E*01:01 | IAPTGHSL | 8 | Septin-6 | 121 |
| HLA-E*01:01 | ISPHGNAL | 8 | ATP-dependent Clp | 122 |
| | | | protease | |
| | | | ATP-binding | |
| | | | subunit clpX-like, | |
| | | | mitochondrial | |
| HLA-E*01:01 | PDPAKSAPAPKKGSK | 15 | Histone H2B type | 123 |
| | | | 1-H | |
| HLA-E*01:01 | PDPAKSAPAPK | 11 | Histone H2B type | 124 |
| | | | 1-H | |
| HLA-E*01:01 | PELAKSAPAPK | 11 | Histone H2B type | 125 |
| | | | 1-L | |
| HLA-E*01:01 | PEPVKSAPVPK | 11 | Histone H2B type | 126 |
| | | | 1-M | |
| HLA-E*01:01 | VAPEEHPVLLTEAPLNPK | 18 | Actin, cytoplasmic 1 | 127 |
| HLA-E*01:03 | RMPPLGHEL | 9 | Mycobacterium | 128 |
| | | | tuberculosis | |
| HLA-E*01:03 | VLRPGGHFL | 9 | Mycobacterium | 129 |
| | | | tuberculosis | |
| HLA-E*01:03 | VMATRRNVL | 9 | Mycobacterium | 130 |
| | | | tuberculosis | |

In some embodiments, the antigen-presenting polypeptide comprises an HLA-G polypeptide and is bound (e.g., specifically bound) to an exogenous antigenic polypeptide. In some embodiments, the antigen-presenting polypeptide comprises an HLA-G polypeptide, and the exogenous antigenic polypeptide has the motif XI/LPXXXXXL (SEQ ID NO: 8).

One of ordinary skill the art could identify an HLA-E- or HLA-G-binding peptide using search tools known in the art, such as, but not limited to, the T cell epitope prediction tools available via Nature at the world wide web at nature.com/articles/2404787/tables/1. International Patent Application Publication No. WO2018/005559, the contents of which are hereby incorporated herein by reference, describes methods of identifying binding peptides for HLA-E and HLA-G.

In some embodiments, the exogenous antigenic polypeptide is between about 8 and about 24 amino acids in length.

In other embodiments, the exogenous antigenic polypeptide presented on an HLA-E or HLA-G antigen-presenting polypeptide is an antigenic polypeptide selected from any one of the antigens disclosed herein. For example, in some embodiments, the polypeptide is an antigenic polypeptide from an antigen selected from the antigens disclosed in Table 1 or has the motif XI/LPXXXXXL (SEQ ID NO: 8).

In some embodiments, the exogenous antigenic polypeptide is derived from an infectious disease agent (e.g., a virus, a parasite (e.g., an intracellular parasite), a prion, a bacterium (e.g., an intracellular pathogenic bacterium). In some embodiments, the exogenous antigenic polypeptide is derived from a virus (e.g., an Epstein Barr virus or HIV). In some embodiments, the exogenous antigenic polypeptide is derived from a bacterium (e.g., Mycobacterium tuberculosis).

In some embodiments, the exogenous antigenic polypeptide is 8 amino acids in length to 24 amino acids in length, for example 8, 9, 10, 11, 12, 13, 14, 15, 16, 17, 18, 19, 20, 21, 22, 23, or 24 amino acids in length. In further embodiments, a cleavable site is introduced into the exogenous antigenic polypeptide.

In some embodiments, an engineered erythroid cell (e.g., engineered enucleated erythroid cell) or enucleated cell (e.g., modified enucleated cell) provide herein comprises at least two types of exogenous polypeptides: at least one (one, two, three, or more) loadable exogenous antigen-presenting polypeptide comprising either an HLA-E polypeptide or an HLA-G polypeptide, and at least one (one, two, three, or more) exogenous antigenic polypeptide (e.g. a first and/or a second exogenous antigenic polypeptide). In some embodiments, a portion of the exogenous antigenic polypeptide is capable of binding to the antigen-binding cleft of the exogenous antigen-presenting polypeptide. In some embodiments, the at least one exogenous antigenic polypeptide(s) comprises a transmembrane domain such as a Type I membrane protein transmembrane domain (e.g., a glycophorin A (GPA) transmembrane domain), or a Type II membrane protein transmembrane domain (e.g., a small integral membrane protein 1 (SMIM1) transmembrane domain), as either an N-terminal or C-terminal fusion, e.g., such that the portion of the antigenic polypeptide that is capable of binding to a exogenous antigen-presenting polypeptide described herein is present on the outer side of the surface of the engineered erythroid cell or enucleated cell. In some embodiments, the exogenous antigenic polypeptide comprises a transmembrane domain, a linker, and an amino acid sequence (e.g., an antigen) that is capable of binding to the antigen-binding cleft of a exogenous antigen-presenting polypeptide. Any of the linkers provided herein may be disposed between the transmembrane domain and the amino acid sequence that is capable of binding to the antigen-binding cleft of the exogenous antigen-presenting polypeptide. For example, in some embodiments, the linker is a flexible linker (e.g., a GlySer linker). In some embodiments, the linker is from about 30 to about 100 amino acid residues in length. In other embodiments, the linker is between about 40 amino acid residues in length and 70 amino acids in length. In some embodiments, the linker is a cleavable linker (e.g., comprising an enzymatic cleavage site). In some embodiments, the exogenous antigenic polypeptide may be covalently-linked to an exogenous antigen-presenting polypeptide. In some embodiments, the exogenous antigenic polypeptide is not covalently-linked to an exogenous antigen-presenting polypeptide.

In some embodiments, an engineered erythroid cell (e.g., engineered enucleated erythroid cell) or enucleated cell (e.g., modified enucleated cell) described herein comprises three or more, e.g., at least 4, 5, 6, 7, 8, 9, 10, 11, 12, 13, 14, 15, 16, 17, 18, 19, 20, 50, 100, 200, 500, or 1000 exogenous antigenic polypeptides. In some embodiments, a population of engineered erythroid cells (e.g., engineered enucleated erythroid cells) or enucleated cells (e.g., modified enucleated cells) described herein comprises three or more, e.g., at least 4, 5, 6, 7, 8, 9, 10, 11, 12, 13, 14, 15, 16, 17, 18, 19, 20, 50, 100, 200, 500, 1000, 2000, or 5000 exogenous antigenic polypeptides, e.g., wherein different engineered erythroid cells (e.g., engineered enucleated erythroid cells) or enucleated cells (e.g., modified enucleated cells) in the population comprise different exogenous antigenic polypeptides or wherein different erythroid cells in the population comprise different pluralities of exogenous antigenic polypeptides.

In some embodiments, the exogenous antigenic polypeptide(s) comprises an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of a corresponding wild-type exogenous antigenic polypeptide.

Nucleic acids (e.g., an exogenous nucleic acid) comprising or consisting of a nucleic acid sequence encoding an exogenous antigenic polypeptide described herein are also provided. In some embodiments, the nucleic acid comprises at least one promoter (e.g., a constitutive or an inducible promoter) operably-linked to the open reading frame or gene encoding the exogenous antigenic polypeptide. In some embodiments, the exogenous antigenic polypeptide is encoded by a nucleic acid (e.g., an exogenous nucleic acid) comprising or consisting of a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence encoding a wild-type exogenous antigenic polypeptide. In some embodiments, the exogenous antigenic polypeptide is encoded by a nucleic acid (e.g., an exogenous nucleic acid) comprising or consisting of a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence encoding a wild-type exogenous antigenic polypeptide, wherein the exogenous antigenic polypeptide does not include a signal sequence. In some embodiments, the nucleic acid is codon-optimized (e.g., for expression in a human cell). In some embodiments, the nucleic acid is not codon-optimized.

In some embodiments, the exogenous antigenic polypeptide is a homolog or variant of a wild-type naturally occurring exogenous antigenic polypeptide. For instance, in some embodiments, the amino acid sequence of an exogenous antigenic polypeptide may differ from the amino acid sequence of a wild-type exogenous antigenic polypeptide reference at one or more amino acid residues. In some embodiments, the amino acid sequence of an exogenous antigenic polypeptide is modified as compared to the amino acid sequence of a wild-typeexogenous antigenic polypeptide to include a conservative (e.g., structurally-similar) amino acid substitution. For instance, structurally similar amino acids include: (isoleucine (I), leucine (L) and valine (V)); (phenylalanine (F) and tyrosine (Y)); (lysine (K) and arginine (R)); (glutamine (Q) and asparagine (N)); (aspartic acid (D) and glutamic acid (E)); and (glycine (G) and alanine (A)). In some embodiments, the amino acid sequence of an exogenous antigenic polypeptide is modified as compared to the amino acid sequence of a wild-type exogenous antigenic polypeptide to include a non-conservative amino acid substitution. In some embodiments, the exogenous antigenic polypeptide includes a deletion, addition, or substitution of at least one amino acid residue as compared to the amino acid sequence of a wild-type exogenous antigenic polypeptide. Homologs and variants of a wild-type exogenous antigenic polypeptide which may be used as described herein include polymorphic variants, natural or artificial mutants, and modified polypeptides in which one or more residues is modified. For example, in some embodiments, the exogenous antigenic polypeptide comprises an amino acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to a wild-type exogenous antigenic polypeptide. In some embodiments, the exogenous antigenic polypeptide amino acid sequence differs from a wild-type exogenous antigenic polypeptide amino acid sequence (e.g., by truncation, deletion, substitution, or addition) by no more than 1, 2, 3, 4, 5, 8, 10, 20, or 50 residues, and retains a function of the wild-type exogenous antigenic polypeptide from which it was derived.

In some embodiments, fragments or variants of an exogenous antigenic polypeptide comprise at least 25%, at least 30%, at least 40%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 853%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% of the exogenous antigenic polypeptide activity of the wild-type exogenous antigenic polypeptide from which the fragment or variant was derived.

### Exogenous Antigen-Presenting Polypeptides

Exogenous antigen-presenting polypeptides of the present disclosure include either a HLA-E polypeptide or a HLA-G polypeptide. In some embodiments, the exogenous antigen-presenting polypeptides of the present disclosure include subunits of a cell surface complex comprising an HLA-E or HLA-G polypeptide, where the HLA-E or HLA-G polypeptide functions to bind an exogenous antigenic polypeptide. In some embodiments, the exogenous antigen-presenting polypeptides are subunits (e.g., a domains) of HLA-E or HLA-G and a function is to bind an exogenous antigenic polypeptide. In some embodiments, the exogenous antigenic polypeptide is loaded on (e.g., to an antigen-binding cleft) the HLA-E or HLA-G polypeptide of the exogenous antigen-presenting polypeptide, and a function of the HLA-E or HLA-G polypeptide is to present the exogenous antigenic polypeptide.

The exogenous antigenic polypeptide may be bound either covalently or non-covalently to the antigen-presenting polypeptide.

In some embodiments, the exogenous antigen-presenting polypeptide comprises a functional HLA-E or HLA-G polypeptide, and the exogenous antigen-presenting polypeptide includes one or more of alpha domains 1-3 (alpha1, alpha2, and alpha3 domains), or fragments or variants thereof. In some embodiments, the exogenous antigen-presenting polypeptide includes beta-2 microglobulin (β2M) polypeptide, or fragments or variants thereof. In some embodiments, the one or more HLA-E or HLA-G a subunits (e.g., one or more a domains) are bound, e.g., covalently bound or non-covalently bound, to a β2M polypeptide. In some embodiments, the one or more HLA-E or HLA-G a subunits (e.g., one or more a domains) are not bound, e.g., covalently bound or non-covalently bound, to a β2M polypeptide.

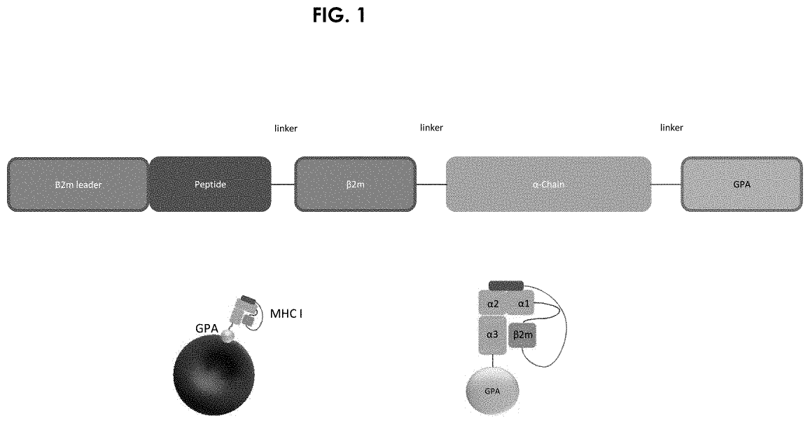

In some embodiments, the antigen-presenting polypeptide comprises a HLA-E polypeptide. In some embodiments, the HLA-E polypeptide is of an HLA-E allele selected from E*01:01, E*01:01:01:01, E*01:01:01:02, E*01:01:01:03, E*01:01:01:04, E*01:01:01:05, E*01:01:01:06, E*01:01:01:07, E*01:01:01:08, E*01:01:01:09, E*01:01:01:10, E*01:01:02, E*01:03, E*01:03:01:01, E*01:03:01:02, E*01:03:01:03, E*01:03:01:04, E*01:03:02:01, E*01:03:02:02, E*01:03:03, E*01:03:04, E*01:03:05, E*01:04, E*01:05, E*01:06, E*01:07, E*01:08N, E*01:09, and E*01:10 allele, or a fragment thereof (e.g., one or more a domains thereof). In some embodiments, the HLA-E polypeptide is linked to (e.g., fused to) an exogenous antigenic polypeptide. For example, in some embodiments, the exogenous antigen presenting polypeptide comprising a HLA-E molecule linked to the exogenous antigenic polypeptide has the structure set forth in FIG. 1. In some embodiments, the exogenous antigenic polypeptide comprises HSP60 or a peptide derived from HSP60 (e.g., a leader sequence of HSP60). In some embodiments, the exogenous antigenic polypeptide comprises a peptide listed in Table 1. In some embodiments, the exogenous antigen-presenting polypeptide comprises an HLA-E polypeptide of a E*01:01, E*01:01:01:01, E*01:01:01:02, E*01:01:01:03, E*01:01:01:04, E*01:01:01:05, E*01:01:01:06, E*01:01:01:07, E*01:01:01:08, E*01:01:01:09, E*01:01:01:10, E*01:01:02, E*01:03, E*01:03:01:01, E*01:03:01:02, E*01:03:01:03, E*01:03:01:04, E*01:03:02:01, E*01:03:02:02, E*01:03:03, E*01:03:04, E*01:03:05, E*01:04, E*01:05, E*01:06, E*01:07, E*01:08N, E*01:09 or E*01:10 allele, and the exogenous antigenic polypeptide comprises a peptide listed in Table 1.

In some embodiments, the exogenous antigen-presenting polypeptide comprises one or more alpha domains 1-3 (e.g., alpha1, alpha2, and alpha3 domains) of an HLA-E polypeptide and a β2M polypeptide. In some embodiments, the exogenous antigen-presenting polypeptide comprises one or more alpha domains 1-3 (e.g., alpha1, alpha2, and alpha3 domains) of HLA-E, a β2M polypeptide, and a membrane anchor (e.g., a GPA protein or a transmembrane domain thereof). In some embodiments, the exogenous antigen-presenting polypeptide comprises one or more alpha domains 1-3 (e.g., alpha1, alpha2, and alpha3 domains) of HLA-E, a β2M polypeptide, a membrane anchor (e.g., a GPA protein or a transmembrane domain thereof), and one or more linkers (e.g., a flexible linker). In some embodiments, the exogenous antigen-presenting polypeptide comprises one or more alpha domains 1-3 (e.g., alpha1, alpha2, and alpha3 domains) of HLA-E, a β2M polypeptide, a membrane anchor (e.g., a GPA protein or a transmembrane domain thereof), and one or more linkers (e.g., a flexible linker), and is linked to an exogenous antigenic polypeptide (e.g., via a linker).

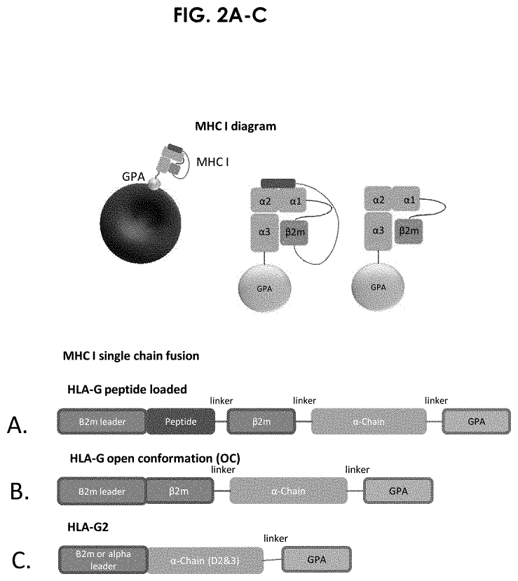

In some embodiments, the antigen-presenting polypeptide comprises an HLA-G polypeptide. In some embodiments, the HLA-G polypeptide is an HLA-G1, HLA-G2, HLA-G3, HLA-G4, HLA-G5, HLA-G6, or HLA-G7 isoform, or a fragment thereof (e.g., one or more a domains thereof). In some embodiments, the HLA-G is an HLA-G multimer, e.g., a dimer. In some embodiments, the HLA-G polypeptide is of the HLA-G1*01:01:01:01 allele. In some embodiments, the HLA-G comprises an unpaired cysteine at residue 42. In some embodiments, the HLA-G polypeptide is linked to an exogenous antigenic polypeptide. In some embodiments, the exogenous antigenic polypeptide has the motif XI/LPXXXXXL (SEQ ID NO: 8), wherein X can be any amino acid. For example, in some embodiments, the exogenous antigen presenting polypeptide comprising a HLA-G polypeptide linked to the exogenous antigenic polypeptide has the structure set forth in FIG. 2A. In other embodiments, the exogenous antigen presenting polypeptide comprising a HLA-G polypeptide has the structure set forth in FIG. 2B. In other embodiments, the exogenous antigen presenting polypeptide comprising a HLA-G polypeptide has the structure set forth in FIG. 2C.

In some embodiments, the exogenous antigen-presenting polypeptide comprises one or more alpha domains 1-3 (e.g., alpha1, alpha2, and alpha3 domains) of a HLA-G polypeptide and does not include a β2M polypeptide. In some embodiments, the exogenous antigen-presenting polypeptide comprises one or more alpha domains 1-3 (e.g., alpha1, alpha2, and alpha3 domains) of a HLA-G polypeptide and a β2M polypeptide. In some embodiments, the exogenous antigen-presenting polypeptide comprises one or more alpha domains 1-3 (e.g., alpha1, alpha2, and alpha3 domains) of a HLA-G polypeptide, and a membrane anchor (e.g., a GPA protein or a transmembrane domain thereof). In some embodiments, the exogenous antigen-presenting polypeptide comprises one or more alpha domains 1-3 (e.g., alpha1, alpha2, and alpha3 domains) of a HLA-G polypeptide, a β2M polypeptide, and a membrane anchor (e.g., a GPA protein or a transmembrane domain thereof). In some embodiments, the exogenous antigen-presenting polypeptide comprises one or more alpha domains 1-3 (e.g., alpha1, alpha2, and alpha3 domains) of a HLA-G polypeptide, a membrane anchor (e.g., a GPA protein or a transmembrane domain thereof), and one or more linkers (e.g., a flexible linker). In some embodiments, the exogenous antigen-presenting polypeptide comprises one or more alpha domains 1-3 (e.g., alpha1, alpha2, and alpha3 domains) of a HLA-G polypeptide, a β2M polypeptide, a membrane anchor (e.g., a GPA protein or a transmembrane domain thereof), and one or more linkers (e.g., a flexible linker). In some embodiments, the exogenous antigen-presenting polypeptide comprises one or more alpha domains 1-3 (e.g., alpha1, alpha2, and alpha3 domains) of an HLA-G polypeptide, a membrane anchor (e.g., a GPA protein or a transmembrane domain thereof), and one or more linkers (e.g., a flexible linker), and is linked to an exogenous antigenic polypeptide (e.g., via a linker). In some embodiments, the exogenous antigen-presenting polypeptide comprises one or more alpha domains 1-3 (e.g., alpha1, alpha2, and alpha3 domains) of a HLA-G polypeptide, a β2M polypeptide, a membrane anchor (e.g., a GPA protein or a transmembrane domain thereof), and one or more linkers (e.g., a flexible linker), and is linked to an exogenous antigenic polypeptide (e.g., via a linker).

In some embodiments, the exogenous antigen-presenting polypeptide comprises alpha1, alpha2, and alpha3 domains of a HLA-G1 isoform polypeptide and does not include a β2M polypeptide. In some embodiments, the exogenous antigen-presenting polypeptide comprises alpha1, alpha2, and alpha3 domains of a HLA-G1 isoform polypeptide and a β2M polypeptide. In some embodiments, the exogenous antigen-presenting polypeptide comprises alpha1, alpha2, and alpha3 domains of a HLA-G1 isoform polypeptide, and a membrane anchor (e.g., a GPA protein or a transmembrane domain thereof). In some embodiments, the exogenous antigen-presenting polypeptide comprises alpha1, alpha2, and alpha3 domains of a HLA-G1 isoform polypeptide, a β2M polypeptide, and a membrane anchor (e.g., a GPA protein or a transmembrane domain thereof). In some embodiments, the exogenous antigen-presenting polypeptide comprises alpha1, alpha2, and alpha3 domains of a HLA-G1 isoform polypeptide, a membrane anchor (e.g., a GPA protein or a transmembrane domain thereof), and one or more linkers (e.g., a flexible linker). In some embodiments, the exogenous antigen-presenting polypeptide comprises alpha1, alpha2, and alpha3 domains of a HLA-G1 isoform polypeptide, a β2M polypeptide, a membrane anchor (e.g., a GPA protein or a transmembrane domain thereof), and one or more linkers (e.g., a flexible linker). In some embodiments, the exogenous antigen-presenting polypeptide comprises alpha1, alpha2, and alpha3 domains of an HLA-G1 isoform polypeptide, a membrane anchor (e.g., a GPA protein or a transmembrane domain thereof), and one or more linkers (e.g., a flexible linker), and is linked to an exogenous antigenic polypeptide (e.g., via a linker). In some embodiments, the exogenous antigen-presenting polypeptide comprises alpha1, alpha2, and alpha3 domains of a HLA-G1 isoform polypeptide, a β2M polypeptide, a membrane anchor (e.g., a GPA protein or a transmembrane domain thereof), and one or more linkers (e.g., a flexible linker), and is linked to an exogenous antigenic polypeptide (e.g., via a linker).

In some embodiments, the exogenous antigen-presenting polypeptide comprises alpha1 and alpha3 domains of a HLA-G2 isoform polypeptide and does not include a β2M polypeptide. In some embodiments, the exogenous antigen-presenting polypeptide comprises alpha1 and alpha3 domains of a HLA-G2 isoform polypeptide and a β2M polypeptide. In some embodiments, the exogenous antigen-presenting polypeptide comprises alpha1 and alpha3 domains of a HLA-G2 isoform polypeptide, and a membrane anchor (e.g., a GPA protein or a transmembrane domain thereof). In some embodiments, the exogenous antigen-presenting polypeptide comprises alpha1 and alpha3 domains of a HLA-G2 isoform polypeptide, a β2M polypeptide, and a membrane anchor (e.g., a GPA protein or a transmembrane domain thereof). In some embodiments, the exogenous antigen-presenting polypeptide comprises alpha1 and alpha3 domains of a HLA-G2 isoform polypeptide, a membrane anchor (e.g., a GPA protein or a transmembrane domain thereof), and one or more linkers (e.g., a flexible linker). In some embodiments, the exogenous antigen-presenting polypeptide comprises alpha1 and alpha3 domains of a HLA-G2 isoform polypeptide, a β2M polypeptide, a membrane anchor (e.g., a GPA protein or a transmembrane domain thereof), and one or more linkers (e.g., a flexible linker). In some embodiments, the exogenous antigen-presenting polypeptide comprises alpha1 and alpha3 domains of an HLA-G2 isoform polypeptide, a membrane anchor (e.g., a GPA protein or a transmembrane domain thereof), and one or more linkers (e.g., a flexible linker), and is linked to an exogenous antigenic polypeptide (e.g., via a linker). In some embodiments, the exogenous antigen-presenting polypeptide comprises alpha1 and alpha3 domains of a HLA-G2 isoform polypeptide, a β2M polypeptide, a membrane anchor (e.g., a GPA protein or a transmembrane domain thereof), and one or more linkers (e.g., a flexible linker), and is linked to an exogenous antigenic polypeptide (e.g., via a linker).

In some embodiments, the exogenous antigen-presenting polypeptide comprises an alpha1 domain of a HLA-G3 isoform polypeptide and does not include a β2M polypeptide. In some embodiments, the exogenous antigen-presenting polypeptide comprises an alpha1 domain of a HLA-G3 isoform polypeptide and a β2M polypeptide. In some embodiments, the exogenous antigen-presenting polypeptide comprises an alpha1 domain of a HLA-G3 isoform polypeptide, and a membrane anchor (e.g., a GPA protein or a transmembrane domain thereof). In some embodiments, the exogenous antigen-presenting polypeptide comprises an alpha1 domain of a HLA-G3 isoform polypeptide, a β2M polypeptide, and a membrane anchor (e.g., a GPA protein or a transmembrane domain thereof). In some embodiments, the exogenous antigen-presenting polypeptide comprises an alpha1 domain of a HLA-G3 isoform polypeptide, a membrane anchor (e.g., a GPA protein or a transmembrane domain thereof), and one or more linkers (e.g., a flexible linker). In some embodiments, the exogenous antigen-presenting polypeptide comprises an alpha1 domain of a HLA-G3 isoform polypeptide, a β2M polypeptide, a membrane anchor (e.g., a GPA protein or a transmembrane domain thereof), and one or more linkers (e.g., a flexible linker). In some embodiments, the exogenous antigen-presenting polypeptide comprises an alpha1 domain of an HLA-G3 isoform polypeptide, a membrane anchor (e.g., a GPA protein or a transmembrane domain thereof), and one or more linkers (e.g., a flexible linker), and is linked to an exogenous antigenic polypeptide (e.g., via a linker). In some embodiments, the exogenous antigen-presenting polypeptide comprises an alpha1 domain of a HLA-G3 isoform polypeptide, a β2M polypeptide, a membrane anchor (e.g., a GPA protein or a transmembrane domain thereof), and one or more linkers (e.g., a flexible linker), and is linked to an exogenous antigenic polypeptide (e.g., via a linker).

In some embodiments, the exogenous antigen-presenting polypeptide comprises alpha1 and alpha2 domains of a HLA-G4 isoform polypeptide and does not include a β2M polypeptide. In some embodiments, the exogenous antigen-presenting polypeptide comprises alpha1 and alpha2 domains of a HLA-G4 isoform polypeptide and a β2M polypeptide. In some embodiments, the exogenous antigen-presenting polypeptide comprises alpha1 and alpha2 domains of a HLA-G4 isoform polypeptide, and a membrane anchor (e.g., a GPA protein or a transmembrane domain thereof). In some embodiments, the exogenous antigen-presenting polypeptide comprises alpha1 and alpha2 domains of a HLA-G4 isoform polypeptide, a β2M polypeptide, and a membrane anchor (e.g., a GPA protein or a transmembrane domain thereof). In some embodiments, the exogenous antigen-presenting polypeptide comprises alpha1 and alpha2 domains of a HLA-G4 isoform polypeptide, a membrane anchor (e.g., a GPA protein or a transmembrane domain thereof), and one or more linkers (e.g., a flexible linker). In some embodiments, the exogenous antigen-presenting polypeptide comprises alpha1 and alpha2 domains of a HLA-G4 isoform polypeptide, a β2M polypeptide, a membrane anchor (e.g., a GPA protein or a transmembrane domain thereof), and one or more linkers (e.g., a flexible linker). In some embodiments, the exogenous antigen-presenting polypeptide comprises alpha1 and alpha2 domains of an HLA-G4 isoform polypeptide, a membrane anchor (e.g., a GPA protein or a transmembrane domain thereof), and one or more linkers (e.g., a flexible linker), and is linked to an exogenous antigenic polypeptide (e.g., via a linker). In some embodiments, the exogenous antigen-presenting polypeptide comprises alpha1 and alpha2 domains of a HLA-G4 isoform polypeptide, a β2M polypeptide, a membrane anchor (e.g., a GPA protein or a transmembrane domain thereof), and one or more linkers (e.g., a flexible linker), and is linked to an exogenous antigenic polypeptide (e.g., via a linker).

In some embodiments, the exogenous antigen-presenting polypeptide comprises alpha1, alpha2, and alpha3 domains of a HLA-G5 isoform polypeptide and does not include a β2M polypeptide. In some embodiments, the exogenous antigen-presenting polypeptide comprises alpha1, alpha2, and alpha3 domains of a HLA-G5 isoform polypeptide and a β2M polypeptide. In some embodiments, the exogenous antigen-presenting polypeptide comprises alpha1, alpha2, and alpha3 domains of a HLA-G5 isoform polypeptide, and a membrane anchor (e.g., a GPA protein or a transmembrane domain thereof). In some embodiments, the exogenous antigen-presenting polypeptide comprises alpha1, alpha2, and alpha3 domains of a HLA-G5 isoform polypeptide, a β2M polypeptide, and a membrane anchor (e.g., a GPA protein or a transmembrane domain thereof). In some embodiments, the exogenous antigen-presenting polypeptide comprises alpha1, alpha2, and alpha3 domains of a HLA-G5 isoform polypeptide, a membrane anchor (e.g., a GPA protein or a transmembrane domain thereof), and one or more linkers (e.g., a flexible linker). In some embodiments, the exogenous antigen-presenting polypeptide comprises alpha1, alpha2, and alpha3 domains of a HLA-G5 isoform polypeptide, a β2M polypeptide, a membrane anchor (e.g., a GPA protein or a transmembrane domain thereof), and one or more linkers (e.g., a flexible linker). In some embodiments, the exogenous antigen-presenting polypeptide comprises alpha1, alpha2, and alpha3 domains of an HLA-G5 isoform polypeptide, a membrane anchor (e.g., a GPA protein or a transmembrane domain thereof), and one or more linkers (e.g., a flexible linker), and is linked to an exogenous antigenic polypeptide (e.g., via a linker). In some embodiments, the exogenous antigen-presenting polypeptide comprises alpha1, alpha2, and alpha3 domains of a HLA-G5 isoform polypeptide, a β2M polypeptide, a membrane anchor (e.g., a GPA protein or a transmembrane domain thereof), and one or more linkers (e.g., a flexible linker), and is linked to an exogenous antigenic polypeptide (e.g., via a linker).

In some embodiments, the exogenous antigen-presenting polypeptide comprises alpha1 and alpha3 domains of a HLA-G6 isoform polypeptide and does not include a β2M polypeptide. In some embodiments, the exogenous antigen-presenting polypeptide comprises alpha1 and alpha3 domains of a HLA-G6 isoform polypeptide and a β2M polypeptide. In some embodiments, the exogenous antigen-presenting polypeptide comprises alpha1 and alpha3 domains of a HLA-G6 isoform polypeptide, and a membrane anchor (e.g., a GPA protein or a transmembrane domain thereof). In some embodiments, the exogenous antigen-presenting polypeptide comprises alpha1 and alpha3 domains of a HLA-G6 isoform polypeptide, a β2M polypeptide, and a membrane anchor (e.g., a GPA protein or a transmembrane domain thereof). In some embodiments, the exogenous antigen-presenting polypeptide comprises alpha1 and alpha3 domains of a HLA-G6 isoform polypeptide, a membrane anchor (e.g., a GPA protein or a transmembrane domain thereof), and one or more linkers (e.g., a flexible linker). In some embodiments, the exogenous antigen-presenting polypeptide comprises alpha1 and alpha3 domains of a HLA-G6 isoform polypeptide, a β2M polypeptide, a membrane anchor (e.g., a GPA protein or a transmembrane domain thereof), and one or more linkers (e.g., a flexible linker). In some embodiments, the exogenous antigen-presenting polypeptide comprises alpha1 and alpha3 domains of an HLA-G6 isoform polypeptide, a membrane anchor (e.g., a GPA protein or a transmembrane domain thereof), and one or more linkers (e.g., a flexible linker), and is linked to an exogenous antigenic polypeptide (e.g., via a linker). In some embodiments, the exogenous antigen-presenting polypeptide comprises alpha1 and alpha3 domains of a HLA-G6 isoform polypeptide, a β2M polypeptide, a membrane anchor (e.g., a GPA protein or a transmembrane domain thereof), and one or more linkers (e.g., a flexible linker), and is linked to an exogenous antigenic polypeptide (e.g., via a linker).

In some embodiments, the exogenous antigen-presenting polypeptide comprises an alpha1 domain of a HLA-G7 isoform polypeptide and does not include a β2M polypeptide. In some embodiments, the exogenous antigen-presenting polypeptide comprises an alpha1 domain of a HLA-G7 isoform polypeptide and a β2M polypeptide. In some embodiments, the exogenous antigen-presenting polypeptide comprises an alpha1 domain of a HLA-G7 isoform polypeptide, and a membrane anchor (e.g., a GPA protein or a transmembrane domain thereof). In some embodiments, the exogenous antigen-presenting polypeptide comprises an alpha1 domain of a HLA-G7 isoform polypeptide, a β2M polypeptide, and a membrane anchor (e.g., a GPA protein or a transmembrane domain thereof). In some embodiments, the exogenous antigen-presenting polypeptide comprises an alpha1 domain of a HLA-G7 isoform polypeptide, a membrane anchor (e.g., a GPA protein or a transmembrane domain thereof), and one or more linkers (e.g., a flexible linker). In some embodiments, the exogenous antigen-presenting polypeptide comprises an alpha1 domain of a HLA-G7 isoform polypeptide, a β2M polypeptide, a membrane anchor (e.g., a GPA protein or a transmembrane domain thereof), and one or more linkers (e.g., a flexible linker). In some embodiments, the exogenous antigen-presenting polypeptide comprises an alpha1 domain of an HLA-G7 isoform polypeptide, a membrane anchor (e.g., a GPA protein or a transmembrane domain thereof), and one or more linkers (e.g., a flexible linker), and is linked to an exogenous antigenic polypeptide (e.g., via a linker). In some embodiments, the exogenous antigen-presenting polypeptide comprises an alpha1 domain of a HLA-G7 isoform polypeptide, a β2M polypeptide, a membrane anchor (e.g., a GPA protein or a transmembrane domain thereof), and one or more linkers (e.g., a flexible linker), and is linked to an exogenous antigenic polypeptide (e.g., via a linker).

An aAPC comprising an engineered erythroid cell (e.g., engineered enucleated erythroid cell) or enucleated cell (e.g., modified enucleated cell), which is engineered to include an exogenous antigen presenting polypeptide comprising a HLA-E or HLA-G polypeptide, as described herein, is used, in some embodiments, for immune modulation, e.g., activation or suppression of one or more immune cell population. In some embodiments, the aAPC comprises a single protein that is a fusion between a HLA-E or HLA-G polypeptide and an exogenous antigenic polypeptide. In some embodiments, a non-membrane tethered component of the complex, e.g., the antigenic polypeptide, or the (32 microglobulin, is assembled with another agent within the cell prior to trafficking to the surface, is secreted by the cell then captured on the surface by the membrane-tethered component of the multimer, or is added in a purified form to an aAPC.

In some embodiments, the exogenous antigen presenting polypeptide comprising a HLA-E or HLA-G polypeptide comprises a membrane anchor, an α-chain (e.g., an a chain of HLA-E or an a chain of HLA-G), and β2M polypeptide. In a further embodiment, the exogenous antigenic polypeptide is connected to the exogenous antigen presenting polypeptide comprising a HLA-E or HLA-G via a linker. In a related embodiment, the linker is a cleavable linker. In another embodiment, the membrane anchor is a glycophorin anchor, and in particular glycophorin A (GPA), or the membrane anchor is small integral membrane protein 1 (SMIM1). In some embodiments, the membrane anchor comprises the full length GPA. In other embodiments, the membrane anchor comprises the transmembrane domain of SMIM1 or the transmembrane domain of GPA.

In other embodiments, the exogenous antigen-presenting polypeptide comprises an HLA-E polypeptide or an HLA-G polypeptide, wherein the amino acid residue corresponding to position 84 of the conserved alpha chain of the mature HLA-E or HLA-G polypeptide comprises an amino acid substitution to an alanine (A) (e.g., Y84A).

In some embodiments, the exogenous antigen-presenting polypeptide is fused to the exogenous antigenic polypeptide as a single chain fusion polypeptide, and the single chain fusion polypeptide comprises (e.g., from N-terminal to C-terminal): an exogenous antigenic polypeptide, a linker, a β2M polypeptide, optionally a linker, one or more alpha domains 1-3 (e.g., alpha1, alpha2, and alpha3 domains) of an HLA-E polypeptide, optionally a linker, and a membrane anchor (e.g., GPA or the transmembrane domain thereof). In some embodiments, the HLA-E polypeptide comprises an amino acid substitution to cysteine at the amino acid residue corresponding to position 84 of the conserved alpha chain of the mature HLA-E polypeptide (e.g., Y84C), and the linker disposed between the exogenous antigenic polypeptide and the β2M polypeptide comprises at least one cysteine residue capable of forming a disulfide bond with the cysteine at the amino acid residue corresponding to position 84 of the conserved alpha chain of the mature HLA-E polypeptide. In some embodiments, the linker disposed between the exogenous antigenic polypeptide and the β2M polypeptide comprises at least one cysteine residue at the first, second, third, fourth, fifth, sixth, seventh, eighth, ninth, tenth, eleventh, or twelfth residue following the terminal end (e.g., N- or C-terminal end) of the exogenous antigenic polypeptide. In some embodiments, the linker disposed between the exogenous antigenic polypeptide and the β2M polypeptide comprises a cysteine residue at the second residue following the terminal end (e.g., N- or C-terminal end) of the exogenous antigenic polypeptide.

In some embodiments, the exogenous antigen-presenting polypeptide is fused to the exogenous antigenic polypeptide as a single chain fusion polypeptide, and the single chain fusion polypeptide comprises (e.g., from N-terminal to C-terminal): an exogenous antigenic polypeptide, a linker, a β2M polypeptide, optionally a linker, one or more alpha domains 1-3 (e.g., alpha1, alpha2, and alpha3 domains) of an HLA-G polypeptide, optionally a linker, and a membrane anchor (e.g., GPA or the transmembrane domain thereof). In some embodiments, the HLA-G polypeptide comprises an amino acid substitution to cysteine at the amino acid residue corresponding to position 84 of the conserved alpha chain of the mature HLA-G polypeptide (e.g., Y84C), and the linker disposed between the exogenous antigenic polypeptide and the β2M polypeptide comprises at least one cysteine residue capable of forming a disulfide bond with the cysteine at the amino acid residue corresponding to position 84 of the conserved alpha chain of the mature HLA-G polypeptide. In some embodiments, the linker disposed between the exogenous antigenic polypeptide and the β2M polypeptide comprises at least one cysteine residue at the first, second, third, fourth, fifth, sixth, seventh, eighth, ninth, tenth, eleventh, or twelfth residue following the terminal end (e.g., N- or C-terminal end) of the exogenous antigenic polypeptide. In some embodiments, the linker disposed between the exogenous antigenic polypeptide and the β2M polypeptide comprises a cysteine residue at the second residue following the terminal end (e.g., N- or C-terminal end) of the exogenous antigenic polypeptide. In some embodiments, the exogenous antigen-presenting polypeptide comprising a HLA-E polypeptide comprises the structure set forth in FIG. 1. In some embodiments, the exogenous antigen-presenting polypeptide comprising a HLA-G polypeptide comprises the structure set forth in any one of FIGS. 2A-2C.

The present disclosure also encompasses exogenous antigen-presenting polypeptide comprising HLA-E or HLA-G polymorphs.

In certain embodiments, the polypeptide is an exogenous antigen-presenting polypeptide as described herein.

### Exogenous Costimulatory Polypeptides

In some embodiments, the aAPCs described herein include an exogenous costimulatory polypeptide. An exogenous costimulatory polypeptide includes a polypeptide on an antigen presenting cell (e.g., an aAPC) that specifically binds a cognate costimulatory molecule on an immune cell, thereby providing a signal which mediates an immune cell response, including, but not limited to, proliferation, activation, differentiation, and the like. A costimulatory polypeptide also encompasses, inter alia, an antibody that specifically binds with a costimulatory molecule present on an immune cell. Such antibody preferably binds and acts as an agonist to the costimulatory molecule on the immune cell.

In some embodiments, the costimulatory polypeptides stimulate or activate immune, e.g., CD8+ T regulatory cells (Tregs). In some embodiments, the aAPC comprising, inter alia, costimulatory polypeptides, promotes immune cell proliferation. In some embodiments, one or more (e.g., 2, 3, 4, or 5 or more) costimulatory polypeptides comprise an activating polypeptide of Table 2, below, or an immune cell activating variant (e.g., fragment) thereof. In some embodiments, one or more (e.g., 2, 3, 4, or 5 or more) costimulatory polypeptides comprise an antibody molecule (e.g. agonizing antibody) that binds a target receptor of Table 2 or an immune cell activating variant (e.g., fragment) thereof. In some embodiments, the costimulatory polypeptides comprise different immune cell activation ligands, e.g., one or more activating polypeptides of Table 2, in any combination thereof, to stimulate or activate immune cells, e.g., CD8+ T regulatory cells (Tregs). In some embodiments, the aAPC comprises an engineered erythroid cell (e.g., engineered enucleated erythroid cell) or enucleated cell (e.g., modified enucleated cell) that presents 4-1BBL, OX40L, and CD40L, or fragments or variants thereof. In some embodiments, these proteins signal through complementary activation pathways. The costimulatory polypeptides can be derived from endogenous immune cell activation ligands or from antibody molecules to the target receptors.

In some embodiments, the costimulatory polypeptide is linked to a transmembrane domain, e.g., the transmembrane domain of an exogenous or endogenous protein. In some embodiments, the costimulatory polypeptide is present on the cell surface of the engineered erythroid cell (e.g., engineered enucleated erythroid cell) or enucleated cell (e.g., modified enucleated cell).

TABLE 2
Costimulatory Polypeptides

| Activating Polypeptide (Ligand) | Target Receptor on T cell |
|---|---|
| B7-H2 (e.g., Accession Number | ICOS, CD28 (e.g., Accession Number |
| NP_056074.1) | NP_006130.1) |
| B7-1 (e.g., Accession Number | CD28 (e.g., Accession Number |
| NP_005182.1) | NP_006130.1) |
| B7-2 (e.g., Accession Number | CD28 (e.g., Accession Number |
| AAA86473) | NP_006130.1) |
| CD70 (e.g., Accession Number | CD27 (e.g., Accession Number |
| NP_001243.1) | NP_001233.1) |
| LIGHT (e.g., Accession Number | HVEM (e.g., Accession Number |
| NP_003798.2) | AAQ89238.1) |
| HVEM (e.g., Accession Number | LIGHT (e.g., Accession Number |
| AAQ89238.1) | NP_003798.2) |
| CD40L (e.g., Accession Number | CD40 (e.g., Accession Number |
| BAA06599.1) | NP_001241.1) |
| 4-1BBL (e.g., Accession Number | 4-1BB (e.g., Accession NP_001552.2) |
| NP_003802.1) | |
| OX40L (e.g., Accession Number | OX40 (e.g., Accession Number |
| NP_003317.1) | NP_003318.1) |
| TL1A (e.g., Accession Number | DR3 (e.g., Accession Number |
| NP_005109.2) | NP_683866.1) |
| GITRL (e.g., Accession Number | GITR (e.g., Accession Number |
| NP_005083.2) | NP_004186.1) |
| CD30L (e.g., Accession Number | CD30 (e.g., Accession Number |
| NP_001235.1), | NP_001234.3) |
| TIM4 (e.g., Accession Number | TIM1 (e.g., Accession Number |
| NP_612388.2) | NP_036338.2) |
| SLAM (e.g., Accession Number | SLAM (e.g., Accession Number |
| AAK77968.1) | AAK77968.1) |
| CD48 (e.g., Accession Number | CD2 (e.g., Accession Number |
| CAG33293.1) | NP_001315538.1) |
| CD58 (e.g., Accession Number | CD2 (e.g., Accession Number |
| CAG33220.1) | NP_001315538.1) |
| CD155 (e.g., Accession Number | CD226 (e.g., Accession Number |
| NP_001129240.1) | NP_006557.2) |
| CD112 (e.g., Accession Number | CD226 (e.g., Accession Number |
| NP_001036189.1) | NP_006557.2) |
| CD137L (e.g., Accession Number | CD137 (e.g., Accession NP_001552.2) |
| NP_003802.1) | |

In some embodiments, the one or more costimulatory polypeptides comprises an activating cytokine, interferon, Ig superfamily member, TNF superfamily receptor, or TNF family member, e.g., IFNα, IL2, IL6 or any combination thereof.

Activating cytokines, interferons, Ig superfamily members, TNF superfamily receptors, or TNF family members which are useful in the present disclosure are discussed further below. In some embodiments, the one or more costimulatory polypeptides comprises one or more activating cytokine, interferon or TNF family member, and further comprises one or more activating polypeptide or ligand (e.g., of Table 2) or an immune cell activating variant (e.g., fragment) thereof, or one or more antibody molecules (e.g. agonizing antibody) that binds a target costimulatory immune cell receptor (e.g., of Table 2) or an immune cell activating variant (e.g., fragment) thereof.

Immune Cell Expansion

In certain embodiments, the disclosure features aAPCs that can be used to specifically induce proliferation of an immune cell, e.g., CD8+ T regulatory cell (Treg), including a known costimulatory molecule. The method comprises contacting an immune cell that is to be expanded with an aAPC presenting an exogenous polypeptide that specifically binds with the costimulatory molecule expressed by the immune cell. Thus, contacting an immune cell with an aAPC comprising, among other things, a costimulatory ligand that specifically binds a cognate costimulatory molecule expressed on the immune cell surface, stimulates the immune cell and induces immune cell proliferation such that large numbers of specific immune cells can be readily produced. The aAPC expands the immune cell “specifically” in that only the immune cells expressing the particular costimulatory molecule are expanded by the aAPC. Thus, where the immune cell to be expanded is present in a mixture of cells, some or most of which do not express the costimulatory molecule, only the immune cell of interest will be induced to proliferate and expand in cell number. The immune cell can be further purified using a wide variety of cell separation and purification techniques, such as those known in the art and/or described elsewhere herein.

As would be appreciated by the skilled artisan, based upon the disclosure provided herein, the immune cell of interest need not be identified or isolated prior to expansion using the aAPC. This is because the aAPC is selective and will only expand the immune cell(s) expressing the cognate costimulatory molecule.

In certain embodiments, the polypeptide is an exogenous costimulatory polypeptide as described herein.

In some embodiments, an aAPC cell targets multiple immune cell activating pathways in combination (e.g., as described in Table 2, above), e.g., using ligands or antibody molecules, or both, co-presented on an aAPC.

In some embodiments, the at least one exogenous costimulatory polypeptide is selected from the group consisting of CD40, ICOS-L, IL-15, 4-1BBL, LIGHT, CD80, CD86, CD70, OX40L, GITRL, TIM4, SLAM, CD48, CD58, CD83, CD155, CD112, IL-15Rα fused to IL-15, IL-2, IL-21, a ligand for ICAM-1, a ligand for LFA-1, and combinations thereof. In some embodiments, the at least one exogenous costimulatory polypeptide is an agonist antibody to the cognate costimulatory ligand receptor. For example, in certain embodiments, the costimulatory polypeptide is an agonist antibody to 4-1-BB, LIGHT receptor (HVEM), CD80 receptor, CD86 receptor, OX40, GITR, TIM4 receptor (TIM1), SLAM receptor, CD48 receptor (CD2), CD58 receptor (CD2), CD 83 receptor, CD155 receptor (CD226), CD112 receptor (CD226), IL-2 receptor (CD25, CD122, CD132), IL-21 receptor, ICAM, and combinations thereof. In certain embodiments, the at least one exogenous costimulatory polypeptide is IL-15. In certain embodiments, the at least one exogenous costimulatory polypeptides is an anti CD3 antibody or an anti-CD38 antibody and combinations thereof. In some embodiments, the aAPC presents at least two, at least 3, at least 4, or at least 5 exogenous costimulatory polypeptides. In some embodiments, the costimulatory proteins are fused to each other, for example IL-21 fused to IL-2.

### Exogenous Coinhibitory Polypeptides

In certain embodiments, the aAPCs described herein include an exogenous coinhibitory polypeptide. An exogenous coinhibitory polypeptide is any polypeptide that suppresses an immune cell, including inhibition of immune cell activity, inhibition of immune cell proliferation, anergizing of an immune cell, or induction of apoptosis of an immune cell.

In some embodiments, an exogenous coinhibitory polypeptide is an inhibitory polypeptide ligand on an antigen presenting cell that specifically binds a cognate coinhibitory molecule on an immune cell. In some embodiments, the coinhibitory polypeptide ligand is an inhibitory polypeptide shown in Table 3.

In some embodiments, the coinhibitory polypeptide is linked to a transmembrane domain, e.g., the transmembrane domain of an exogenous or endogenous protein. In some embodiments, the coinhibitory polypeptide is present on the cell surface of the engineered erythroid cell (e.g., engineered enucleated erythroid cell) or enucleated cell (e.g., modified enucleated cell).

In some embodiments, an exogenous coinhibitory polypeptide is an agonist (e.g. an antibody) that specifically binds a coinhibitory receptor on an immune cell, e.g., a T cell, a B cell, a macrophage, DC, or an NK. In some embodiments, the agonist is an antibody that binds a receptor selected from the group consisting of: PD1, CTLA4, TIM3, TGFβ, CEACAM (e.g., CEACAM-1, CEACAM-3 and/or CEACAM-5), LAG3, VISTA, BTLA, TIGIT, LAIR1, CD160, and 2B4. In other embodiments, the agonist is an antibody that binds a target receptor on an immune cell, e.g., a T cell, a B cell, a macrophage, DC, or an NK cell shown in Table 3.

In some embodiments, an exogenous coinhibitory polypeptide is a checkpoint molecule on the cell surface. In some embodiments, the checkpoint molecule is selected from the group consisting of PD-L1, PD-L2, and OX40L. In some embodiments, an exogenous coinhibiotry polypeptide is an agonist of PD-1, CTLA4, TIM3, or LAG3.

TABLE 3
Coinhibitory Polypeptides

| Inhibitory Polypeptide | Target Receptor on T cell |
|---|---|
| B7-1 | CTLA4, B7H1 |
| B7-2 | CTLA4 |
| B7DC | PD1 |
| B7H1 | PD1, B7-1 |
| HVEM | CD160, BTLA |
| COLLAGEN | LAIR1 |
| GALECTIN9 | TIM3 |
| CD48, TIM4 | TIM4R |
| CD48 | 2B4 |
| CD155, CD112, CD113 | TIGIT |
| PDL 1 | PD1 |
| | LAG3 |

In some embodiments, an exogenous coinhibitory polypeptide is an antibody that blocks binding of a costimulatory polypeptide to its cognate costimulatory receptor. In various embodiments, the exogenous coinhibitory polypeptide is an antibody that blocks binding of 4-1BBL, LIGHT, CD80, CD86, CD70, OX40L, GITRL, TIM4, SLAM, CD48, CD58, CD83, CD155, CD112, IL-15Rα fused to IL-15, IL-2, IL-21, ICAM, a ligand for LFA-1, an anti CD3 antibody or an anti CD28 antibody, to its receptor.

In other embodiments, the coinhibitory polypeptide is selected from IL-35, IL-10, or VSIG-3.

In other embodiments, an aAPC cell targets multiple immune cell inhibitory pathways in combination (e.g., as described in Table 3, above), e.g., using ligands or antibody molecules, or both, on an aAPC.

In certain embodiments, the polypeptide is an exogenous coinhibitory polypeptide as described herein.

In some embodiments, the aAPC presents at least two, at least 3, at least 4, or at least 5 exogenous coinhibitory polypeptides.

### Treg Costimulatory and Coinhibitory Polypeptides

Regulatory T cells (“Treg”) are a specialized subpopulation of T cells which suppresses activation of the immune system and thereby maintains tolerance to self-antigens. Treg cells constitute 5-10% of CD4⁺ T cells in humans and rodents. Treg cells constitute 5-10% of CD4⁺ T cells in humans and rodents, and constitutively express CD4 and CD25, as well as the transcription factor FoxP3 (CD4+CD25+FoxP3+), which is involved in their development and function. IL-2 also appears to play an important role in Treg cell development and homeostatsis because animals deficient for IL-2 or components of its receptor develop T cell hyperproliferation and autoimmune diseases that can be corrected by adoptive transfer of Treg cells from naive animals. Similarly, a lack of signaling through CD28/CD80 interaction is associated with reduced number and functionality of Treg cells, suggesting that this receptor/ligand system plays an important role in the development and function of Treg cells.

In certain embodiments, the aAPCs described herein include an exogenous Treg costimulatory polypeptide. In certain embodiments, the present disclosure features aAPCs comprising Treg costimulatory polypeptides that are capable of expanding regulatory T-cells (Tregs) cells. In some embodiments, the Treg costimulatory polypeptides expand Treg cells by stimulating at least one of three signals involved in Treg cell development. Signal 1 involves TCR, and can be stimulated with antibodies, such as anti-CD3 antibodies, or with antigens that signals through TCR. Signal 2 can be mediated by several different molecules, including immune costimulatory molecules such as CD80 and 4-1BBL. Signal 3 is transduced via cytokines, such as IL-2, or TGFβ. In some embodiments, the Treg costimulatory polypeptides stimulate one of these signals. In some embodiments, the Treg costimulatory polypeptides stimulate two of these signals. In yet another embodiment, the Treg costimulatory polypeptides stimulate three of these signals.

Signal 1

Antigens useful as Treg costimulatory polypeptides for stimulating Signal 1 include antigens associated with a target disease or condition. For example, autoantigens and insulin (particularly suitable for treating type 1 diabetes), collagen (particularly suitable for treating rheumatoid arthritis), myelin basic protein (particularly suitable for treating multiple sclerosis) and MHC (for treating and preventing foreign graft rejection). The antigens may be administered as part of a conjugate. Optionally, the antigen is provided as part of an HLA-E or HLA-G/antigen complex. In this embodiment, the HLA-E or HLA-G and antigen can independently be foreign or syngenic. For example donor HLA-E or HLA-G and an allogenic or syngenic antigen can be used.

Signal 2

Exemplary Treg costimulatory polypeptides for stimulating Signal 2 include members of the B7 and TNF families, for example B7 and CD28 family members, shown below in Table 4, and TNF family members shown in Table 5.

TABLE 4
Treg Costimulatory Polypeptides: B7 and CD28 Family Members

| LIGAND | RECEPTOR |
|---|---|
| B7.1 (CD80) | CD28, CTLA-4 (CD152) |
| B7.2 (CD86) | CD28, CTLA-4 |
| ICOSL (B7h, B7-H2, B7RP-1, | ICOS (AILIM) |
| GL5O, LICOS) | |
| PD-L1 (B7-H1) | PD-1 |
| PD-L2 (B7-DC) | PD-1 |
| B7-H3 | Unknown |
| B7-H4 (B7x; B7S1) | Unknown (BTLA?) |
| Unknown (HVEM*) | BTLA |
| ICOSL (B7h, B7-H2, B7RP-1, | ICOS (AILIM) |

TABLE 5
Treg Costimulatory Polypeptides: TNF Family Members

| LIGAND | RECEPTOR |
|---|---|
| OX40L | OX40 (CD134) |
| 4-1BBL | 4-1BB (CD137) |
| CD40L (CD154) | CD40 |
| CD27L (CD70) | CD27 |
| CD30L | CD30 |
| LIGHT | HVEM, LTβR, DcR3 |
| GITRL | GITR |
| BAFF (BLyS) ** | BAFF-R, TACT, BCMA |
| APRIL ** | TACT, BCMA |
| VEGI/TL1A | DR3 |
| TNF alpha (mutants) | TNFR2 |

Signal 3

Exemplary Treg costimulatory polypeptides for stimulating Signal 3 include cytokines and growth factors that stimulate Signal 3, such as IL-2, IL-4, and TGF-β (including TGF-β1, TGF-β2 and TGF-β3). IL-2 and IL-4 moieties useful in immunotherapeutic methods are known in the art. See, e.g., Earle et al., 2005, supra; Thorton et al., 2004, J. Immunol. 172: 6519-23; Thorton et al., 2004, Eur. J. Immunol. 34: 366-76. In accordance with some embodiments, the mature portion of the cytokine is used.

In some embodiments, the Treg costimulatory polypeptide is IL-2, e.g., CD25-specific IL-2. In some embodiments, the Treg costimulatory polypeptide is TNF, e.g., TNFR2-specific TNF. In some embodiments, the Treg costimulatory polypeptide is an anti-DR3 agonist (VEGI/TL1A specific). In some embodiments, the Treg costimulatory peptide is 41BBL. In some embodiments, the Treg costimulatory peptide is TGFbeta. In some embodiments, the Treg costimulatory peptide is CD80. In some embodiments, the Treg costimulatory peptide is CD86.

In other embodiments, the present disclosure features Treg coinhibtory polypeptides that are exogenous polypeptides that inhibit Treg cells. In certain embodiments, Treg inhibition is useful in the treatment of cancer, for example, by targeting chemokines that are involved in Treg trafficking. Other Treg inhibitors can target any of the receptors listed in Tables 4 or 5, for example, anti-OX40, anti-GITR or anti-CTLA4, or TLR ligands.

In certain embodiments, the polypeptide is an exogenous Treg costimulatory polypeptide as described herein.

In some embodiments, the aAPC presents at least two, at least 3, at least 4, or at least 5 exogenous Treg costimulatory polypeptides.

In some embodiments, the Treg costimulatory polypeptide or Treg coinhibitory polypeptide is linked to a transmembrane domain, e.g., the transmembrane domain of an endogenous protein. In some embodiments, the Treg costimulatory polypeptide or Treg coinhibitory is present on the cell surface of the engineered erythroid cell (e.g., engineered enucleated erythroid cell) or enucleated cell (e.g., modified enucleated cell).

### Cytokines/Chemokines

In certain embodiments, the aAPCs described herein include at least one exogenous polypeptide comprising a cytokine, a chemokine, or both. Additionally, the disclosure encompasses an aAPC transduced with a nucleic acid encoding at least one cytokine, at least one chemokine, or both. Thus, the disclosure provides aAPCs comprising an exogenous polypeptide comprising a cytokine, including a full-length, fragment, homologue, variant or mutant of the cytokine. A cytokine includes a protein that is capable of affecting the biological function of another cell. A biological function affected by a cytokine can include, but is not limited to, cell growth, cell differentiation or cell death. Preferably, a cytokine of the present disclosure is capable of binding to a specific receptor on the surface of a cell, thereby affecting the biological function of a cell.

A preferred cytokine includes, among others, a hematopoietic growth factor, an interleukin, an interferon, an immunoglobulin superfamily molecule, a tumor necrosis factor family molecule and/or a chemokine. A more preferred cytokine of the disclosure includes a granulocyte macrophage colony stimulating factor (GM-CSF), tumor necrosis factor alpha (TNFα), tumor necrosis factor beta (TNFβ), macrophage colony stimulating factor (M-CSF), interleukin-1 (IL-1), interleukin-2 (IL-2), interleukin-4 (IL-4), interleukin-5 (IL-5), interleukin-6 (IL-6), interleukin-7 (IL-7), interleukin-10 (IL-10), interleukin-12 (IL-12), interleukin-15 (IL-15), interleukin-21 (IL-21), interleukin-35 (IL-35), interferon alpha (IFN-α), interferon beta (IFN-β), interferon gamma (IFN-γ), and IGIF, among many others. In some embodiments, the aAPC includes the antigen-presenting polypeptide HLA-E and is transduced with IL-15.

In some embodiments, the disclosure provides aAPCs comprising an exogenous polypeptide comprising a chemokine, including a homologue, variant, mutant or fragment thereof, such as an alpha-chemokine or a beta-chemokine, including, but not limited to, a C5a, interleukin-8 (IL-8), monocyte chemotactic protein 1 alpha (MIP 1α), monocyte chemotactic protein 1 beta (MIP1β), monocyte chemoattractant protein 1 (MCP-1), monocyte chemoattractant protein 3 (MCP-3), platelet activating factor (PAFR), N-formyl-methionyl-leucyl-[³H]phenylalanine (FMLPR), leukotriene B₄ (LTB₄R), gastrin releasing peptide (GRP), RANTES, eotaxin, lymphotactin, IP10, 1-309, ENA78, GCP-2, NAP-2 and/or MGSA/gro. One skilled in the art would appreciate, once armed with the teachings provided herein, that the disclosure encompasses a APCs comprising an exogenous polypeptide comprising a chemokine and/or a cytokine, such as those well-known in the art, as well as any discovered in the future.

In some embodiments, an engineered erythroid cell (e.g., engineered enucleated erythroid cell) or enucleated cell (e.g., modified enucleated cell) comprises one or more (e.g., 2, 3, 4, 5, or more) cytokine receptor subunits from Table 6 or cytokine-binding variants or fragments thereof. In some embodiments, an engineered erythroid cell (e.g., engineered enucleated erythroid cell) or enucleated cell (e.g., modified enucleated cell) comprises two or three (e.g., all) cytokine receptor subunits from a single row of Table 4 or cytokine-binding variants or functional fragments thereof. The cytokine receptors can be present on the surface of the engineered erythroid cell (e.g., engineered enucleated erythroid cell) or enucleated cell (e.g., modified enucleated cell). The expressed receptors typically have the wild type human receptor sequence or a variant or fragment thereof that is able to bind and sequester its target ligand. In some embodiments, two or more cytokine receptor subunits are linked to each other, e.g., as a fusion protein.

In some embodiments, one or more (e.g., 2 or all) of the cytokines are fused to a transmembrane domains (e.g., a GPA transmembrane domain or other transmembrane domain described herein), e.g., such that the cytokine is present on the surface of the engineered erythroid cell (e.g., engineered enucleated erythroid cell) or enucleated cell (e.g., modified enucleated cell). In some embodiments, the engineered erythroid cell (e.g., engineered enucleated erythroid cell) or enucleated cell (e.g., modified enucleated cell) further comprises a targeting moiety, e.g., an address moiety or targeting moiety described in WO2007030708, e.g., in pages 34-45 therein, which application is herein incorporated by reference in its entirety.

TABLE 6
Cytokines and Receptors

| Name | Cytokine Receptor(s)(Da) and Form |
|---|---|
| Interleukins | |
| IL-1-like | |
| IL-1α | CD121a, CDw121b |
| IL-1β | CD121a, CDw121b |
| IL-1RA | CD121a |
| IL-18 | IL-18Rα, β |
| Common g chain (CD132) | |
| IL-2 | CD25, 122, 132 |
| IL-4 | CD124, 213a13, 132 |
| IL-7 | CD127, 132 |
| IL-9 | IL-9R, CD132 |
| IL-13 | CD213a1, 213a2, |
| IL-15 | IL-15Ra, CD122, 132 |
| IL-21 | IL21R |
| Common b chain (CD131) | |
| IL-3 | CD123, CDw131 |
| IL-5 | CDw125, 131 |
| Also related | |
| GM-CSF | CD116, CDw131 |
| IL-6-like | |
| IL-6 | CD126, 130 |
| IL-11 | IL-11Ra, CD130 |
| Also related | |
| G-CSF | CD114 |
| IL-12 | CD212 |
| IL-35 | IL35R |
| LIF | LIFR, CD130 |
| OSM | OSMR, CD130 |
| IL-10-like | |
| IL-10 | CDw210 |
| IL-20 | IL-20Rα, β |
| Others | |
| IL-14 | IL-14R |
| IL-16 | CD4 |
| IL-17 | CDw217 |
| Interferons | |
| IFN-α | CD118 |
| IFN-β | CD118 |
| IFN-γ | CDw119 |
| TNF | |
| CD154 | CD40 |
| LT-β | LTβR |
| TNF-α | CD120a, b |
| TNF-β(LT-α) | CD120a, b |
| 4-1BBL | CD137 (4-1BB) |
| APRIL | BCMA, TACI |
| CD70 | CD27 |
| CD153 | CD30 |
| CD178 | CD95 (Fas) |
| GITRL | GITR |
| LIGHT | LTbR, HVEM |
| OX40L | OX40 |
| TALL-1 | BCMA, TACI |
| TRAIL | TRAILR1-4 |
| TWEAK | Apo3 |
| TRANCE | RANK, OPG |
| TGF-β | |
| TGF-β1 | TGF-βR1 |
| TGF-β2 | TGF-βR2 |
| TGF-β3 | TGF-βR3 |
| Miscellaneous | |
| hematopoietins | |
| Epo | EpoR |
| Tpo | TpoR |
| Flt-3L | Flt-3 |
| SCF | CD117 |
| M-CSF | CD115 |
| MSP | CDw136 |

In some embodiments, the Treg costimulatory polypeptides, costimulatory polypeptides, coinhibitory polypeptides, or cytokines described herein, or an active fragment thereof, can be linked or expressed as a fusion protein with a binding pair member for use in accordance with the present disclosure. An exemplary binding pair is biotin and streptavidin (SA) or avidin.

In some embodiments, the Treg costimulatory polypeptides, costimulatory polypeptides, coinhibitory polypeptides, or cytokines, or an active fragment thereof, is part of a fusion protein, comprising a Treg costimulatory polypeptide, costimulatory polypeptide, coinhibitory polypeptide, or cytokine and a binding pair member, such as CSA. Fusion proteins can be made by any of a number of different methods known in the art. For example, one or more of the component polypeptides of the fusion proteins can be chemically synthesized or can be generated using well known recombinant nucleic acid technology.

The conjugate may include a linker such as a peptide linker between the binding pair member and the costimulatory moiety. The linker length and composition may be chosen to enhance the activity of either functional end of the moiety. The linker may be greater than 20 amino acids long. In some embodiments, the linker is generally from about 3 to about 30 amino acids long, for example about 5 to about 20 amino acids long, about 5 to about 15 amino acids long, about a to about 10 amino acids long. However, longer or shorter linkers may be used or the linker may be dispensed with entirely. Flexible linkers (e.g. (Gly4Ser)3) such as have been used to connect heavy and light chains of a single chain antibody may be used in this regard. See, e.g., Huston et al., 1988, Proc. Nat. Acad. Sci. USA, 85: 5879-5883; U.S. Pat. Nos. 5,091,513, 5,132,405, 4,956,778; 5,258,498, and 5,482,858. Other linkers are FENDAQAPKS (SEQ ID NO: 9) or LQNDAQAPKS (SEQ ID NO: 10). One or more domains of an immunoglobulin Fc region (e.g. CH1, CH2 and/or CH3) also may be used as a linker.

### Engineered Erythroid Cells

In some aspects, the present disclosure provides an artificial antigen presenting cell (aAPC) engineered to activate or inhibit certain immune cell populations, wherein the aAPC comprises an engineered erythroid cell (e.g., engineered enucleated erythroid cell) or enucleated cell (e.g., modified enucleated cell) that is engineered to include an exogenous antigen-presenting polypeptide comprising an HLA-E polypeptide or an HLA-G polypeptide, and to activate or suppress specific populations of immune cells. In some embodiments, the cells are engineered to include an exogenous antigen-presenting polypeptide comprising an HLA-E polypeptide, and activate certain immune cells, e.g., T regulatory cells. In some embodiments, the cells are engineered to include an exogenous antigen-presenting polypeptide comprising an HLA-E polypeptide, and suppress certain immune cells, e.g., NK cells and cytotoxic CD8+ T cells. In some embodiments, the cells are engineered to include an exogenous antigen-presenting polypeptide comprising an HLA-G polypeptide, and suppress certain immune cells, e.g., natural killer (NK) cells, T cells, B cells, macrophages, and/or dentritic cells (DC).

In some embodiments, the engineered erythroid cell (e.g., engineered enucleated erythroid cell) or enucleated cell (e.g., modified enucleated cell), includes an exogenous antigen-presenting polypeptide comprising an HLA-E polypeptide or an HLA-G polypeptide, which presents an exogenous antigenic polypeptide disclosed in Table 1 or an exogenous antigenic polypeptide having the motif XI/LPXXXXXL (SEQ ID NO: 8).

In some embodiments, an enucleated cell is a cell that lacks a nucleus (e.g., due to a differentiation process such as erythropoiesis). In some embodiments, an enucleated cell is incapable of expressing a polypeptide. In some embodiments, an enucleated cell is an erythrocyte, a reticulocyte, or a platelet. Engineered erythroid cells (e.g., engineered enucleated erythroid cells) or enucleated cells (e.g., modified enucleated cells) may be advantageously used for the treatment of an autoimmune disease, an inflammatory disease, an infectious disease or an allergic disease. Specifically, the engineered erythroid cells (e.g., engineered enucleated erythroid cells) or enucleated cells (e.g., modified enucleated cells) described herein may be non-autologous (e.g., substantially lack major histocompatibility complex (MHC)), circulate for long periods of time in a subject (e.g., greater than 30 days), and may be produced in large quantities. Moreover, engineered erythroid cells (e.g., engineered enucleated erythroid cells) or enucleated cells (e.g., modified enucleated cells) may be highly deformable rendering the cells capable of accessing sites within a subject that may not be accessed by other therapeutic means.

The engineered erythroid cell (e.g., engineered enucleated erythroid cell) or enucleated cell (e.g., modified enucleated cell) comprises a first exogenous polypeptide. In some embodiments, the engineered erythroid cell (e.g., engineered enucleated erythroid cell) or enucleated cell (e.g., modified enucleated cell) further comprises a second, different, exogenous polypeptide. The engineered erythroid cell (e.g., engineered enucleated erythroid cell) or enucleated cell (e.g., modified enucleated cell) optionally further comprises second and third, different, exogenous polypeptides. The engineered erythroid cell (e.g., engineered enucleated erythroid cell) or enucleated cell (e.g., modified enucleated cell) optionally further comprises second, third and fourth, different, exogenous polypeptides. The engineered erythroid cell (e.g., engineered enucleated erythroid cell) or enucleated cell (e.g., modified enucleated cell) optionally further comprises second, third, fourth and fifth, different, exogenous polypeptides. In some embodiments, the engineered erythroid cell (e.g., engineered enucleated erythroid cell) or enucleated cell (e.g., modified enucleated cell) optionally further comprises 1, 2, 3, 4, 5, 6, 7, 8, 9, 10, 11, 12, 13, 14, 15, 16, 17, 18, 19, 20 or more different, exogenous polypeptides. In some embodiments, the engineered erythroid cell (e.g., engineered enucleated erythroid cell) or enucleated cell (e.g., modified enucleated cell) optionally further comprises between 1-100, 1-200 different, exogenous polypeptides.

In certain embodiments, the engineered erythroid cell (e.g., engineered enucleated erythroid cell) or enucleated cell (e.g., modified enucleated cell) comprising an antigen, can process and/or present the antigen in the context of an exogenous antigen-presenting polypeptide comprising an HLA-E polypeptide or an HLA-G polypeptide, thereby producing antigen-specific T cells and expanding a population thereof. Therefore, an antigen of interest can be introduced into an aAPC of the disclosure, wherein the aAPC then presents the antigen in the context of the an exogenous antigen-presenting polypeptide comprising an HLA-E polypeptide or an HLA-G polypeptide, i.e., the exogenous antigen-presenting polypeptide comprising the HLA-E polypeptide or the HLA-G polypeptide is “loaded” with or bound to the antigen, and the aAPC can be used to produce an antigen-specific T cell. Thus, in some aspects, the present disclosure provides an artificial antigen presenting cell (aAPC) engineered to modulate immune cells, wherein the aAPC comprises an engineered erythroid cell (e.g., engineered enucleated erythroid cell) or enucleated cell (e.g., modified enucleated cell), wherein the cell presents an exogenous antigen-presenting polypeptide comprising an HLA-E polypeptide or an HLA-G polypeptide, and an exogenous antigenic polypeptide.

In other aspects, the present disclosure provides an artificial antigen presenting cell (aAPC) engineered to activate T regulatory cells, wherein the aAPC comprises an engineered erythroid cell (e.g., engineered enucleated erythroid cell) or enucleated cell (e.g., modified enucleated cell), wherein the engineered erythroid cell (e.g., engineered enucleated erythroid cell) or enucleated cell (e.g., modified enucleated cell) presents an exogenous antigenic polypeptide and an exogenous costimulatory polypeptide.

In some aspects, the present disclosure provides, an artificial antigen presenting cell (aAPC) engineered to activate T regulatory cells, wherein the aAPC comprises an engineered erythroid cell (e.g., engineered enucleated erythroid cell) or enucleated cell (e.g., modified enucleated cell), wherein the cell presents an exogenous antigen-presenting polypeptide, e.g., an exogenous antigen-presenting polypeptide comprising an HLA-E polypeptide, and an exogenous antigenic polypeptide, wherein the exogenous antigen-presenting polypeptide is a single chain fusion polypeptide.

In other aspects, the present disclosure provides, an artificial antigen presenting cell (aAPC) engineered to activate and expand T regulatory cells, wherein the aAPC comprises an engineered erythroid cell (e.g., engineered enucleated erythroid cell) or enucleated cell (e.g., modified enucleated cell), wherein the cell includes an exogenous antigen-presenting polypeptide, e.g., an exogenous antigen-presenting polypeptide comprising an HLA-E polypeptide, an exogenous antigenic polypeptide, and an exogenous costimulatory polypeptide, an exogenous T regulatory cell costimulatory polypeptide, and/or an exogenous polypeptide comprising a cytokine.

In some embodiments, the aAPC is capable of stimulating an immune cell contacted with the aAPC. In other embodiments, stimulating comprises activation of CD8+ T cells, activation of CD4+ T cells, stimulation of cytotoxic activity of T cells, stimulation of cytokine secretion by T cells, and/or any combination thereof.

In other aspects, the present disclosure provides an artificial antigen presenting cell (aAPC) engineered to inhibit certain immune cell populations, e.g., natural killer cells, T cells, B cells, macrophages, dentritic cells (DC), etc., wherein the aAPC comprises an engineered erythroid cell (e.g., engineered enucleated erythroid cell) or enucleated cell (e.g., modified enucleated cell), presents an exogenous antigenic polypeptide and an exogenous coinhibitory polypeptide.

In some aspects, the present disclosure provides, an artificial antigen presenting cell (aAPC) engineered to inhibit certain immune cell populations, e.g., natural killer cells, T cells, B cells, macrophages, dentritic cells (DC), etc., wherein the aAPC comprises an engineered erythroid cell (e.g., engineered enucleated erythroid cell) or enucleated cell (e.g., modified enucleated cell), wherein the cell includes an exogenous antigen-presenting polypeptide, e.g., an exogenous antigen-presenting polypeptide comprising a HLA-G polypeptide, and an exogenous antigenic polypeptide, wherein the exogenous antigen-presenting polypeptide is an HLA-E or HLA-G single chain fusion polypeptide. In some embodiments, the single chain fusion polypeptide comprises an exogenous antigenic polypeptide linked to the HLA-E polypeptide or to the HLA-G polypeptide.

In other aspects, the present disclosure provides, an artificial antigen presenting cell (aAPC) engineered to inhibit certain immune cell populations, wherein the aAPC comprises an engineered erythroid cell (e.g., engineered enucleated erythroid cell) or enucleated cell (e.g., modified enucleated cell), wherein the cell presents an exogenous antigen-presenting polypeptide, e.g., an exogenous antigen-presenting polypeptide comprising a HLA-G polypeptide or a HLA-E polypeptide, an exogenous antigenic polypeptide, an exogenous coinhibitory polypeptide, and/or an exogenous polypeptide comprising a cytokine.

In some embodiments, the objective is to activate or to inhibit T cells. To ensure that T cells are preferentially targeted over other immune cells that may also express either activating or inhibitory receptors as described herein, one of the exogenous polypeptides on the engineered erythroid cell (e.g., engineered enucleated erythroid cell) or enucleated cell (e.g., modified enucleated cell) may comprise a targeting moiety, e.g., an antibody molecule that binds the T cell receptor (TCR) or another T cell marker. Targeting moieties are described in more detail herein below. In some embodiments, a specific T cell subtype or clone may be enhanced or inhibited. In some embodiments, one or more of the exogenous polypeptides on the engineered erythroid cell is an exogenous polypeptide comprising a HLA-E or a HLA-G polypeptide that is “loaded” with an antigenic peptide which will selectively bind to a T cell receptor in an antigen-specific manner.

In some aspects, the present disclosure provides an artificial antigen presenting cell (aAPC) engineered to suppress T cell, B cell, NK cell, macrophage, or dendritic cell activity, wherein the aAPC comprises an engineered erythroid cell (e.g., engineered enucleated erythroid cell) or enucleated cell (e.g., modified enucleated cell), wherein the erythroid cell includes an exogenous antigen-presenting polypeptide, e.g., an exogenous antigen-presenting polypeptide comprising a HLA-G polypeptide, an exogenous antigenic polypeptide, and at least one of: an exogenous coinhibitory polypeptide disclosed in Table 3, or an exogenous polypeptide comprising IL10, PDL1, or 4-1BBL.

In other aspects, the present disclosure provides an artificial antigen presenting cell (aAPC) engineered to suppress T cell, B cell, NK cell, macrophage, or dendritic cell activity, wherein the aAPC comprises an engineered erythroid cell (e.g., engineered enucleated erythroid cell) or enucleated cell (e.g., modified enucleated cell), wherein the erythroid cell includes an exogenous antigen-presenting polypeptide, e.g., an exogenous antigen-presenting polypeptide comprising a HLA-G polypeptide, an exogenous antigenic polypeptide, and at least one exogenous coinhibitory polypeptide selected from IL10, PDL1, or 4-1BBL.

In some embodiments, the aAPC is capable of suppressing T cells, B cells, NK cells, macrophages, or dendritic cells contacted with the aAPC. In other embodiments, the aAPC is capable of suppressing a T cell, B cell, NK cell, macrophage, or dendritic cell that interacts with the aAPC. In further embodiments, the suppressing comprises inhibition, anergizing, or induction of apoptosis of a cell.

In some aspects, the present disclosure provides, an artificial antigen presenting cell (aAPC) engineered to activate a regulatory T cell (Treg cell), wherein the aAPC comprises an engineered erythroid cell (e.g., engineered enucleated erythroid cell) or enucleated cell (e.g., modified enucleated cell), wherein the erythroid cell includes an exogenous antigen-presenting polypeptide, e.g., an exogenous antigen-presenting polypeptide comprising a HLA-E polypeptide, and an exogenous antigenic polypeptide. In some embodiments, the aAPC further presents an exogenous Treg expansion polypeptide or an exogenous polypeptide comprising a cytokine, e.g., IL-15.

In some embodiments, the engineered erythroid cell comprises an exogenous polypeptide (e.g., exogenous antigenic polypeptide, exogenous antigen-presenting polypeptide, exogenous costimulatory polypeptide, exogenous coinhibitory polypeptide, cytokine, or exogenous Treg costimulatory polypeptide), wherein the engineered erythroid cell (e.g., engineered enucleated erythroid cell) or enucleated cell (e.g., modified enucleated cell) optionally further comprises a second exogenous polypeptide (e.g., exogenous antigenic polypeptide, exogenous antigen-presenting polypeptide, exogenous costimulatory polypeptide, exogenous coinhibitory polypeptide, cytokine and exogenous Treg costimulatory polypeptide), wherein the second exogenous polypeptide is any exogenous polypeptide described herein.

The present disclosure should also be construed to encompass “mutants,” “derivatives,” and “variants” of the exogenous polypeptides described herein (or of the DNA encoding the same) which mutants, derivatives and variants are costimulatory ligands, cytokines, antigens (e.g., tumor cell, viral, and other antigens), which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of a costimulatory ligand, cytokine, antigen, and the like, of the present disclosure (e.g., inclusion by an aAPC where contacting the aAPC comprising the protein with a T cell, mediates proliferation of, or otherwise affects, the T cell). Any number of procedures may be used for the generation of mutant, derivative or variant forms of a protein of the disclosure using recombinant DNA methodology well known in the art such as, for example, that described in Sambrook and Russell (2001, Molecular Cloning, A Laboratory Approach, Cold Spring Harbor Press, Cold Spring Harbor, N.Y.), and Ausubel et al. (2002, Current Protocols in Molecular Biology, John Wiley & Sons, NY). Procedures for the introduction of amino acid changes in a protein or polypeptide by altering the DNA sequence encoding the polypeptide are well known in the art and are also described in these, and other, treatises.

The skilled artisan would appreciate, once armed with the teachings provided herein, that the aAPC of the disclosure is not limited in any way to any particular exogenous antigenic polypeptide, cytokine, costimulatory polypeptide, antibody that specifically binds a costimulatory molecule, and the like. Rather, the disclosure encompasses an aAPC comprising numerous molecules, either all under the control of a single promoter/regulatory sequence or under the control of more than one such sequence. Moreover, the disclosure encompasses administration of one or more aAPC of the disclosure where the various aAPCs comprise different molecules. That is, the various molecules (e.g., co stimulatory polypeptides, exogenous antigenic polypeptides, cytokines, and the like) can work in cis (i.e., in the same aAPC and/or encoded by the same contiguous nucleic acid or on separate nucleic acid molecules within the same aAPC) or in trans (i.e., the various molecules are included in different aAPCs).

### Circulation Time

In some embodiments, the engineered erythroid cell or enucleated cell (e.g., enucleated erythroid cell) of the present disclosure resides in circulation after administration to a subject for at least about 1 day to about 240 days (e.g., for at least about 1 day, 2 days, 3 days, 4 day, 5 days, 6 days, 7 days, 8 days, 9 days, 10 days, 11 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days, 183 days, 184 days, 185 days, 186 days, 187 days, 188 days, 189 days, 190 days, 191 days, 192 days, 193 days, 194 days, 195 days, 196 days, 197 days, 198 days, 199 days, 200 days, 201 days, 202 days, 203 days, 204 days, 205 days, 206 days, 207 days, 208 days, 209 days, 210 days, 211 days, 212 days, 213 days, 214 days, 215 days, 216 days, 217 days, 218 days, 219 days, 220 days, 221 days, 222 days, 223 days, 224 days, 225 days, 226 days, 227 days, 228 days, 229 days, 230 days, 231 days, 232 days, 233 days, 234 days, 235 days, 236 days, 237 days, 238 days, 239 days, or 240 days.

### Modifications

One or more of the exogenous proteins may include a post-translational modification characteristic of eukaryotic cells, e.g., mammalian cells, e.g., human cells. In some embodiments, one or more (e.g., 2, 3, 4, 5, 6, 7, 8, 9, 10, or more) of the exogenous proteins are glycosylated, phosphorylated, or both. In vitro detection of glycoproteins can be accomplished on SDS-PAGE gels and Western Blots using a modification of Periodic acid-Schiff (PAS) methods. Cellular localization of glycoproteins can be accomplished utilizing lectin fluorescent conjugates known in the art. Phosphorylation may be assessed by Western blot using phospho-specific antibodies.

Post-translation modifications also include conjugation to a hydrophobic group (e.g., myristoylation, palmitoylation, isoprenylation, prenylation, or glypiation), conjugation to a cofactor (e.g., lipoylation, flavin moiety (e.g., FMN or FAD), heme C attachment, phosphopantetheinylation, or retinylidene Schiff base formation), diphthamide formation, ethanolamine phosphoglycerol attachment, hypusine formation, acylation (e.g. O-acylation, N-acylation, or S-acylation), formylation, acetylation, alkylation (e.g., methylation or ethylation), amidation, butyrylation, gamma-carboxylation, malonylation, hydroxylation, iodination, nucleotide addition such as ADP-ribosylation, oxidation, phosphate ester (O-linked) or phosphoramidate (N-linked) formation, (e.g., phosphorylation or adenylylation), propionylation, pyroglutamate formation, S-glutathionylation, S-nitrosylation, succinylation, sulfation, ISGylation, SUMOylation, ubiquitination, Neddylation, or a chemical modification of an amino acid (e.g., citrullination, deamidation, eliminylation, or carbamylation), formation of a disulfide bridge, racemization (e.g., of proline, serine, alanine, or methionine). In embodiments, glycosylation includes the addition of a glycosyl group to arginine, asparagine, cysteine, hydroxylysine, serine, threonine, tyrosine, or tryptophan, resulting in a glycoprotein. In embodiments, the glycosylation comprises, e.g., O-linked glycosylation or N-linked glycosylation.

### Copy Number

In some embodiments, the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, from about 100:1 to 1:100 by weight or by copy number.

In some embodiments, the engineered erythroid cell (e.g., engineered enucleated erythroid cell) or enucleated cell (e.g., modified enucleated cell) comprises at least at least 10 copies, 100 copies, 1,000 copies, 5,000 copies 10,000 copies, 25,000 copies, 50,000 copies, or 100,000 copies of each of the first exogenous polypeptide and the second exogenous polypeptide. In some embodiments, the copy number of the first exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the second exogenous polypeptide. In some embodiments, the copy number of the second exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the first exogenous polypeptide.

In some embodiments, the first exogenous polypeptide comprises between about 50,000 to about 600,000 copies of the first exogenous polypeptide, for example about 50,000, 60,000, 60,000, 80,000, 90,000, 100,000, 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of the first polypeptide. In some embodiments, the engineered erythroid cell (e.g., engineered enucleated erythroid cell) or enucleated cell (e.g., modified enucleated cell) comprises between about 50,000-600,000, between about 100,000-600,000, between about 100,000-500,000, between about 100,000-400,000, between about 100,000-150,000, between about 150,000-300,000, or between 150,000-200,000 copies of the first exogenous polypeptide. In some embodiments, the engineered erythroid cell (e.g., engineered enucleated erythroid cell) or enucleated cell (e.g., modified enucleated cell) comprises at least about 75,000 copies of the first exogenous polypeptide. In some embodiments, the engineered erythroid cell comprises at least about 100,000 copies of the first exogenous polypeptide. In some embodiments, the engineered erythroid cell comprises at least about 125,000 copies of the first exogenous polypeptide. In some embodiments, the engineered erythroid cell comprises at least about 150,000 copies of the first exogenous polypeptide. In some embodiments, the engineered erythroid cell comprises at least about 175,000 copies of the first exogenous polypeptide. In some embodiments, the engineered erythroid cell comprises at least about 200,000 copies of the first exogenous polypeptide. In some embodiments, the engineered erythroid cell comprises at least about 250,000 copies of the first exogenous polypeptide. In some embodiments, the engineered erythroid cell comprises at least about 300,000 copies of the first exogenous polypeptide. In some embodiments, the engineered erythroid cell comprises at least about 400,000 copies of the first exogenous polypeptide. In some embodiments, the engineered erythroid cell comprises at least about 500,000 copies of the first exogenous polypeptide. In some embodiments, the second exogenous polypeptide comprises between about 50,000 to about 600,000 copies of the second exogenous polypeptide, for example about 50,000, 60,000, 60,000, 80,000, 90,000, 100,000, 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of the second polypeptide. In some embodiments, the engineered erythroid cell comprises between about 50,000-600,000, between about 100,000-600,000, between about 100,000-500,000, between about 100,000-400,000, between about 100,000-150,000, between about 150,000-300,000, or between 150,000-200,000 copies of the second exogenous polypeptide. In some embodiments, the engineered erythroid cell comprises at least about 75,000 copies of the second exogenous polypeptide. In some embodiments, the engineered erythroid cell comprises at least about 100,000 copies of the second exogenous polypeptide. In some embodiments, the engineered erythroid cell comprises at least about 125,000 copies of the second exogenous polypeptide. In some embodiments, the engineered erythroid cell comprises at least about 150,000 copies of the second exogenous polypeptide. In some embodiments, the engineered erythroid cell comprises at least about 175,000 copies of the second exogenous polypeptide. In some embodiments, the engineered erythroid cell (e.g., engineered enucleated erythroid cell) or enucleated cell (e.g., modified enucleated cell) comprises at least about 200,000 copies of the second exogenous polypeptide. In some embodiments, the engineered erythroid cell (e.g., engineered enucleated erythroid cell) or enucleated cell (e.g., modified enucleated cell) comprises at least about 250,000 copies of the second exogenous polypeptide. In some embodiments, the engineered erythroid cell comprises at least about 300,000 copies of the second exogenous polypeptide. In some embodiments, the engineered erythroid cell (e.g., engineered enucleated erythroid cell) or enucleated cell (e.g., modified enucleated cell) comprises at least about 400,000 copies of the second exogenous polypeptide. In some embodiments, the engineered erythroid cell (e.g., engineered enucleated erythroid cell) or enucleated cell (e.g., modified enucleated cell) comprises at least about 500,000 copies of the second exogenous polypeptide.

### Gene Editing

In some aspects, the disclosure features a method of making an immunologically compatible artificial antigen presenting cell (aAPC), wherein a suitable cell, e.g., a nucleated erythroid cell, an erythroid precursor cell, or a nucleated platelet precursor cell, expresses an exogenous antigenic polypeptide (e.g., an exogenous antigen-presenting polypeptide comprising a HLA-E polypeptide or a HLA-G polypeptide), the method comprising contacting the cell with a nuclease and at least one gRNA which cleave an endogenous nucleic acid encoding a HLA-E or HLA-G polypeptide, wherein the endogenous nucleic acid encoding a HLA-E or HLA-G polypeptide is modified by a gene editing pathway and results in a decrease in the level of the endogenous HLA-E or HLA-G polypeptide, thereby making the immunologically compatible aAPC.

In some embodiments, a suitable cell, e.g., a nucleated erythroid cell, an erythroid precursor cell, or a nucleated platelet precursor cell is genetically modified using a nuclease that is targeted to one or more selected DNA sequences. Such methods may be used to induce precise cleavage at selected sites in endogenous genomic loci. Genetic engineering in which DNA is inserted, replaced, or removed from a genome, e.g., at a defined location of interest, using targetable nucleases, may be referred to as “genome editing”. Examples of such nucleases include zinc-finger nucleases (ZFNs), Transcription activator-like effector nuclease (TALENs), engineered meganuclease homing endonucleases, and RNA directed nucleases such as CRISPR (clustered regularly interspaced short palindromic repeats)-associated (Cas) nucleases, e.g., derived from type II bacterial CRISPR/Cas systems (e.g., Cas9).

In some embodiments, an alteration is first introduced using CRISPR (i.e. increasing endogenous expression of HLA-E or HLA-G). Then, the antigen for presentation is also introduced via CRISPR and processed internally.

In some embodiments, the nuclease comprises a DNA cleavage domain and a DNA binding domain (DBD) that targets the nuclease to a particular DNA sequence, thereby allowing the nuclease to be used to engineer genomic alterations in a sequence-specific manner. The DNA cleavage domain may create a double-stranded break (DSB) or nick at or near the sequence to which it is targeted. ZFNs comprise DBDs selected or designed based on DBDs of zinc finger (ZF) proteins. DBDs of ZF proteins bind DNA in a sequence-specific manner through one or more zinc fingers, which are regions of amino acid sequence whose structure is stabilized through coordination of a zinc ion. TALENs comprise DBDs selected or designed based on DBDs of transcription activator-like (TAL) effectors (TALEs) of Xanthomonas spp. ZFN or TALEN dimers induce targeted DNA DSBs that stimulate DNA damage response pathways. The binding specificity of the designed zinc-finger domain directs the ZFN to a specific genomic site. TALEs contain multiple 33-35-amino-acid repeat domains, each of which recognizes a single base pair. Like ZFNs, TALENs induce targeted DSBs that activate DNA damage response pathways and enable custom alterations. The DNA cleavage domain of an engineered site-specific nuclease may comprise a catalytic domain from a naturally occurring endonuclease such as the Fokl endonuclease or a variant thereof. In some embodiments Fokl cleavage domain variants with mutations designed to improve cleavage specificity and/or cleavage activity may be used (see, e.g., Guo, J., et al. (2010) Journal of Molecular Biology 400 (1): 96-107; Doyon, Y., et al., (2011) Nature Methods 8: 74-79. Meganucleases are sequence-specific endonucleases characterized by a large recognition site (double-stranded DNA sequences of 12 to about 40 base pairs). The site generally occurs no more than once in a given genome. The specificity of a meganuclease can be changed by introducing changes sequence of the nuclease (e.g., in the DNA binding domain) and then selecting functional enzymes capable of cleaving variants of the natural recognition site or by associating or fusing protein domains from different nucleases.

In some embodiments, an RNA directed nuclease may be used to perform genome editing. For example, the use of CRISPR/Cas-based systems is contemplated. In some embodiments, a Cas nuclease, such as Cas9 (e.g., Cas9 of Streptococcus pyogenes, Streptococcus thermophiles, or Neisseria meningiditis, or a variant thereof), is introduced into cells along with a guide RNA comprising a sequence complementary to a sequence of interest (the RNA is sometimes termed a single guide RNA). The region of complementarity may be, e.g., about 20 nucleotides long. The Cas nuclease, e.g., Cas9, is guided to a particular DNA sequence of interest by the guide RNA. The guide RNA may be engineered to have complementarity to a target sequence of interest in the genome, e.g., a sequence in any gene or intergenic region of interest. The nuclease activity of the Cas protein, e.g., Cas9, cleaves the DNA, which can disable the gene, or cut it apart, allowing a different DNA sequence to be inserted. In some embodiments multiple sgRNAs comprising sequences complementary to different genes, e.g., 2, 3, 4, 5, or more genes, are introduced into the same cell sequentially or together. In some embodiments, alterations in multiple genes may thereby be generated in the same step.

In general, use of nuclease-based systems for genetic engineering, e.g., genome editing, entails introducing a nuclease into cells and maintaining the cells under conditions and for a time appropriate for the nuclease to cleave the cell's DNA.

For CRISP/Cas systems, a guide RNA is also introduced. The nuclease is typically introduced into the cell by introducing a nucleic acid encoding the nuclease. The nucleic acid may be operably linked to a promoter capable of directing expression in the cell and may be introduced into the cell in a plasmid or other vector. In some embodiments mRNA encoding the nuclease may be introduced. In some embodiments, the nuclease itself may be introduced. sgRNA may be introduced directly (by methods such as transfection) or by expressing it from a nucleic acid construct such as an expression vector. In some embodiments, a sgRNA and Cas protein are expressed from a single expression vector that has been introduced into the cell or, in some embodiments, from different expression vectors. In some embodiments multiple sgRNAs comprising sequences complementary to different genes, e.g., 2, 3, 4, 5, or more genes, are introduced into the same cell individually or together as RNA or by introducing one or more nucleic acid constructs encoding the sgRNAs into the cell for intracellular transcription.

Upon cleavage by a nuclease, a target locus (e.g., in the genome of a cell) may undergo one of two major pathways for DNA damage repair, namely non-homologous end joining (NHEJ) or homology-directed repair (HDR). In the absence of a suitable repair template comprising sufficient homology to the sequences flanking the cleavage site to stimulate HDR (see discussion below), DSBs are re-ligated through NHEJ, which can result in an insertion or deletion. NHEJ can be used, for example, to engineer gene knockouts or generate proteins with altered activity. For example, an insertion or deletion in an exon can lead to a frameshift mutation or premature stop codon. Two or more DSBs can be generated in order to produce larger deletions in the genome.

In some embodiments, a nucleic acid (e.g., a plasmid or linear DNA) comprising a sequence of interest to be inserted into the genome at the location of cleavage is introduced into a cell in addition to a nuclease. In some embodiments, a sequence of interest is inserted into a gene. The sequence of interest may at least in part replace the gene. In some embodiments, the nucleic acid comprises sequences that are homologous to the sequences flanking the cleavage site, so that homology-directed repair is stimulated. In some embodiments, the nucleic acid contains a desired alteration as compared to a sequence present in the cell's genome at or near the site of cleavage. A nucleic acid comprising a sequence to be at least in part introduced into the genome, e.g., a nucleic acid sequence comprising homologous sequence(s) and a desired alteration may be referred to as a “donor sequence”. The donor sequence may become at least in part physically into integrated the genome at the site of a break or may be used as a template for repair of the break, resulting in the introduction of all or part of the nucleotide sequence present in the donor into the genome of the cell. Thus, a sequence in a cell's genome can be altered and, in certain embodiments, can be converted into a sequence present in a donor nucleic acid. In some embodiments, the donor sequence may be contained in a circular DNA (e.g. a plasmid), a linear double-stranded DNA (e.g., a linearized plasmid or a PCR product), or single-stranded DNA, e.g., a single-stranded oligonucleotide. In some embodiments, the donor sequence has between about 10-25 bp and about 50-100 bp of homology to either side or each side of the target site in the genome. In some embodiments, a longer homologous sequence may be used, e.g., between about 100-500 bp up to about 1-2 kB, or more. In some embodiments, an alteration is introduced into one allele of a gene. In some embodiments, a first alteration is introduced into one allele of a gene, and a different alteration is introduced into the other allele. In some embodiments, the same alteration is introduced into both alleles. In some embodiments two alleles or target sites (or more) may be genetically modified in a single step. In some embodiments two alleles or target sites (or more) may be genetically modified in separate steps.

Methods of designing, generating and using ZFNs and/or TALENs are described in, e.g., WO2011097036; Urnov, F D, et al., Nature Reviews Genetics (2010), 11: 636-646; Miller J C, et al., Nat Biotechnol. (2011) 29(2): 143-8; Cermak, T., et al. Nucleic Acids Research (2011) 39 (12): e82, Sanjana, N. E. et al. A transcription activator-like effector toolbox for genome engineering. Nat Protoc 7, 171-192 (2012) and references in any of the foregoing. ZFN, TALEN, and CRISPR/Cas-based methods for genome engineering are reviewed in Gaj, T., et al., Trends Biotechnol. 2013 July; 31(7):397-405. Epub 2013 May 9. Use of CRISPR/Cas systems in genome engineering is described in, e.g., Cong L, et al. Multiplex genome engineering using CRISPR/Cas systems. Science. 2013; 339(6121):819-23; Mali P, et al., RNA-guided human genome engineering via Cas9. Science. 2013; 339(6121):823-6; Wang, H. et al. One-step generation of mice carrying mutations in multiple genes by CRISPR/Cas-mediated genome engineering. Cell 153, 910-918 (2013); Ran, F. A. et al. Double Nicking by RNA-Guided CRISPR Cas9 for Enhanced Genome Editing Specificity. Cell 154, 1380-1389 (2013); Mali, P., et al., Nat Methods. 2013; 10(10):957-63; Ran, FA, Nat Protoc. 2013; 8(11):2281-308). In some embodiments, a nuclease that cleaves only one strand of dsDNA (a nickase) may be used to stimulate HDR without activating the NHEJ repair pathway. Nickases may be created by inactivating the catalytic activity of one nuclease monomer in the ZFN or TALEN dimer required for double stranded cleavage or inactivating a catalytic domain of a Cas protein. For example, mutations of one of the catalytic residues (D10 in the RuvC nuclease domain and H840 in the HNH nuclease domain), e.g., to alanines (D10A, H840A) convert Cas9 into DNA nickases.

In some embodiments, a CRISP/Cas based system may be used to modulate gene expression. For example, coexpression of a guide RNA with a catalytically inactive Cas9 lacking endonuclease activity generates a DNA recognition complex that can specifically interfere with transcriptional elongation, RN A polymerase binding, or transcription factor binding. This system, sometimes referred to CRISPR interference (CRISPRi), can efficiently repress expression of targeted genes in mammalian cells (Qi, S., et al., Cell, 2013; 152(5): 1173-83; Larson, M H, et al, Nat Protoc. 2013; 8(11):2180-96). By attaching any of a variety of effector domains to a catalytically inactive Cas9 one can create a chimeric Cas9 protein that can be used to achieve sequence-specific control over gene expression and/or DNA modification. Suitable effector domains include, e.g., a transcriptional activation domain (such as those comprising the VP16 transactivation domain, e.g., VP64), a transcriptional coactivation domain, a transcriptional inhibitory or coinhibitory domain, a protein-protein interaction domain, an enzymatic domain, etc. A guide RNA guides the chimeric Cas9 protein to a site of interest in the genome (e.g., in or near an expression control element such as a promoter), whereby the effector domain exerts an effect such as activating or inhibiting transcriptional activity (see, e.g., Gilbert L A, et al. Cell. 2013; 154(2):442-51; Maeder M L, et al., Nat Methods, 2013; 10(10):977-9), Appropriate effector domains may be any of those present in naturally occurring proteins that are capable of performing the function of interest (e.g., inhibiting or activating transcription).

Cells that have been subjected to a genetic engineering process may be selected or analyzed to identify or isolate those that express a desired recombinant gene product or lack expression of an endogenous gene that has been disabled via genetic engineering or have any desired genetic alteration. For example, in some embodiments, the donor sequence or vector used to deliver the donor sequence may comprise a selectable marker, which may be used to select cells that have incorporated at least a portion of the donor sequence comprising the selectable marker into their genome. In some embodiments selection is not used. In some embodiments cells may be screened, e.g., by Southern blot to identify those cells or clones that have a desired genetic alteration. If desired, cells may be tested for expression level or activity of a recombinant gene product or endogenous gene product or for one or more functional properties associated with or conferred by a recombinant or endogenous gene product, or any other criteria of interest. Suitable methods of analysis are known to those of ordinary skill in the art and include, e.g., Western blot, flow cytometry, FAGS, immunofluorescence microscopy, ELISA assays, affinity-based methods in which cells are contacted with an agent capable of binding to a protein of interest that labels or retains cells that express the protein, etc. Functional assays may be selected based on the identity of the recombinant gene product, endogenous gene product, and/or function or property of interest. For example, a functional property may be ability to bind to an antigen of interest or ability to exert cytotoxicity towards target cells that express an antigen of interest. Cells may be analyzed, e.g., by PGR, Southern blotting, or sequencing, to determine the number of inserted DNA sequences, their location, and/or to determine whether desired genomic alterations have occurred. One or more cells that have desired alteration(s), expression level, and/or functional properties may be identified, propagated, expanded. The cells or their descendants may be used to generate a cell line, subjected to sortagging, and/or stored for future use.

### Populations of Engineered Erythroid Cells

In one aspect, the disclosure features populations of the engineered erythroid cells or enucleated cells described herein e.g., a plurality or population of the engineered enucleated erythroid cells. The terms “plurality” and “population” are used interchangeably herein. In some embodiments, a population of engineered erythroid cells or enucleated cells may comprise predominantly enucleated cells (e.g., greater than 70%), predominantly nucleated cells (e.g., greater than 70%), or any mixture of enucleated and nucleated cells. In some embodiments, a population of engineered erythroid cells or enucleated cells may comprise reticulocytes, erythrocytes, or a mixture of reticulocytes and erythrocytes. In some embodiments, a population of engineered erythroid cells or enucleated cells may predominantly comprise reticulocytes. In some embodiments, a population of engineered erythroid cells or enucleated cells may predominantly comprise erythrocytes (e.g., immature or mature erythrocytes).

In some embodiments, a population of engineered erythroid cells consists essentially of enucleated cells. In some embodiments, a population of engineered erythroid cells comprises predominantly or substantially enucleated cells. For example, in some embodiments, a population of engineered erythroid cells comprises at least about 70% or more enucleated cells. In some embodiments, the population provided herein comprises at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99, or about 100% enucleated cells. In some embodiments, the population provided herein comprises greater than about 70% enucleated cells. In some embodiments, the population of engineered erythroid cells comprises greater than about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% enucleated cells. In some embodiments, the population of engineered erythroid cells comprises between about 80% and about 100% enucleated cells, for example between about 80% and about 95%, about 80% and about 90%, about 80% and about 85%, about 85% and about 100%, about 85% and about 95%, about 85% and about 90%, about 90% and about 100%, about 90% and about 95%, or about 95% and about 100% of enucleated cells.

In some embodiments, the population of engineered erythroid cells comprises less than about 30% nucleated cells. For example, in embodiments, the population of engineered erythroid cells comprises less than about 1%, about 2%, about 3%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or less than about 30% nucleated cells. In some embodiments, the population of engineered erythroid cells comprises less than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, or about 19%, about 21%, about 22%, about 23%, about 24%, about 25%, about 26%, about 27%, about 28%, about 29%, or about 30% nucleated cells. In some embodiments, the population of engineered erythroid cells comprises between 0% and 2030% nucleated cells. In some embodiments, the populations of engineered erythroid cells comprise between about 0% and 20% nucleated cells, for example between about 0% and 19%, between about 0% and 15%, between about 0% and 10%, between about 0% and 5%, between about 0% and 4%, between about 0% and 3%, between about 0% and 2% nucleated cells, or between about 5% and 20%, between about 10% and 20%, or between about 15% and 20% nucleated cells.

In some embodiments, the disclosure features a population of the engineered erythroid cells as described herein, wherein the population of engineered erythroid cells comprises less than 30% nucleated cells and at least 70% enucleated cells, or comprises less than 20% nucleated cells and at least 80% enucleated cells, or comprises less than 15% nucleated cells and at least 85% nucleated cells, or comprises less than 10% nucleated cells and at least 90% enucleated cells, or comprises less than 5% nucleated cells and at least 95% enucleated cells. In some embodiments, the disclosure features populations of the engineered erythroid cells as described herein, wherein the population of engineered erythroid cells comprises about 0% nucleated cells and about 100% enucleated cells, about 1% nucleated cells and about 99% enucleated cells, about 2% nucleated cells and about 98% enucleated cells, about 3% nucleated cells and about 97% enucleated cells, about 4% nucleated cells and about 96% enucleated cells, about 5% nucleated cells and about 95% enucleated cells, about 6% nucleated cells and about 94% enucleated cells, about 7% nucleated cells and about 93% enucleated cells, about 8% nucleated cells and about 92% enucleated cells, about 9% nucleated cells and about 91% enucleated cells, about 10% nucleated cells and about 90% enucleated cells, about 11% nucleated cells and about 89% enucleated cells, about 12% nucleated cells and about 88% enucleated cells, about 13% nucleated cells and about 87% enucleated cells, about 14% nucleated cells and about 86% enucleated cells, about 85% nucleated cells and about 85% enucleated cells, about 16% nucleated cells and about 84% enucleated cells, about 17% nucleated cells and about 83% enucleated cells, about 18% nucleated cells and about 82% enucleated cells, about 19% nucleated cells and about 81% enucleated cells, or about 20% nucleated cells and about 80% enucleated cells.

In another embodiment, the engineered erythroid cell population comprises predominantly or substantially nucleated cells. In some embodiments, the engineered erythroid cell population consists essentially of nucleated cells. In various embodiments, the nucleated cells in the engineered erythroid cell population are erythroid precursor cells. In some embodiments, the erythroid precursor cells are selected from the group consisting of pluripotent hematopoietic stem cells (HSCs), multipotent myeloid progenitor cells, CFU-S cells, BFU-E cells, CFU-E cells, pronormoblasts, basophilic normoblasts, polychromatophilic normoblasts and orthochromatophilic normoblasts.

In certain embodiments, the population of engineered erythroid cells comprises at least about 10%, at least about 20%, at least about 30%, at least about 40%, at least about 50%, at least about 60%, at least about 70%, at least about 75%, at least about 80%, at least about 85%, at least about 90%, at least about 95%, at least about 98%, at least about 99% or 100% nucleated cells.

It will be understood that during the preparation of the engineered erythroid cells or enculeated cells of the as described herein, some fraction of cells may not include an exogenous polypeptide (e.g., due to lack of expression or transduction or conjugation with an exogenous nucleic acid). Accordingly, in some embodiments, a population of engineered erythroid cells or enucleated cells provided herein comprises a mixture of engineered erythroid cells and unmodified erythroid cells, or a mixture of modified enucleated cells and unmodified enucleated cells, i.e., some fraction of cells in the population will not include (e.g., express) an exogenous polypeptide. For example, a population of engineered erythroid cells or enucleated cells can comprise, in various embodiments, at least about 10%, 20%, 30%, 40%, 50%, 60%, 70%, 80%, 90%, 91%, 92%, 93%, 94%, 95%, 96%, 97%, 98% or 99% erythroid cells or enucleated cells that include an exogenous polypeptide, wherein the remaining erythroid cells or enucleated cells in the population are do not include an exogenous polypeptide. In some embodiments, a single unit dose of engineered erythroid cells (e.g., engineered enucleated erythroid cells) or enucleated cells comprises at least about 10%, 20%, 30%, 40%, 50%, 60%, 65%, 70%, 75%, 80%, 85%, 90%, 91%, 92%, 93%, 94%, 95%, 96%, 97%, 98% or 99% erythroid cells or enucleated cells including an exogenous polypeptide, wherein the remaining erythroid cells or enucleated cells in the dose do not include an exogenous polypeptide.

### III. Methods of Making Artificial Antigen Presenting Cells

Various methods of making aAPCs are contemplated by the present disclosure. In one aspect, the present disclosure features a method of making an immunologically compatible artificial antigen presenting cell (aAPC), wherein the aAPC comprises an engineered erythroid cell (e.g., engineered enucleated erythroid cell) or enucleated cell (e.g., modified enucleated cell) that presents an exogenous antigenic polypeptide, the method comprising contacting a nucleated cell with a nuclease and at least one gRNA which cleave an endogenous nucleic acid to result in expression of an endogenous antigen-presenting polypeptide, an endogenous membrane anchor polypeptide, or an endogenous costimulatory or coinhibitory polypeptide; introducing an exogenous nucleic acid encoding the exogenous antigenic polypeptide into the nucleated cell; and culturing the nucleated cell under conditions suitable for expression and presentation of the exogenous antigenic polypeptide by the endogenous antigen-presenting polypeptide, and enucleation, thereby making an enucleated cell, thereby making the immunologically compatible aAPC. Methods of making an aAPC are described herein, however it is to be understood that these methods are non-limiting.

The processes of making the engineered erythroid cells and enucleated cells are described in more detail below.

### Methods of Manufacturing Enucleated Erythroid Cells

Methods of manufacturing enucleated erythroid cells comprising an exogenous agent (e.g., a polypeptide) are described, e.g., in International Application Publication Nos. WO2015/073587 and WO2015/153102, each of which is incorporated by reference in its entirety.

In some embodiments, hematopoietic progenitor cells, e.g., CD34⁺ hematopoietic progenitor cells (e.g., human (e.g., adult human) or mouse cells), are contacted with a nucleic acid or nucleic acids encoding one or more exogenous polypeptides, and the cells are allowed to expand and differentiate in culture. In some embodiments, the CD34⁺ cells are immortalized, e.g., comprise a human papilloma virus (HPV; e.g., HPV type 16) E6 and/or E7 genes. In some embodiments, the immortalized CD34⁺ hematopoietic progenitor cell is a BEL-A cell line cell (see Trakarnasanga et al. (2017) Nat. Commun. 8: 14750). Additional immortalized CD34⁺ hematopoietic progenitor cells are described in U.S. Pat. Nos. 9,951,350, and 8,975,072. In some embodiments, an immortalized CD34⁺ hematopoietic progenitor cell is contacted with a nucleic acid or nucleic acids encoding one or more exogenous polypeptides, and the cells are allowed to expand and differentiate in culture.

In some embodiments, the erythroid cells described herein are made by a method comprising contacting a nucleated erythroid cell (e.g., an erythroid precursor cell) with an exogenous nucleic acid. In some embodiments, the exogenous nucleic acid is codon-optimized. For instance, the exogenous nucleic acid may comprise one or more codons that differ from the wild-type codons in a way that does not change the amino acid encoded by that codon, but that increases translation of the nucleic acid, e.g., by using a codon preferred by the host cell, e.g., a mammalian cell, e.g., an erythroid cell.

The exogenous nucleic acid may be, e.g., DNA or RNA (e.g., mRNA). A number of viruses may be used as gene transfer vehicles including retroviruses, Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example.

In some embodiments, the exogenous nucleic acid is operatively linked to a constitutive promoter. In some embodiments, a constitutive promoter is used to drive expression of the targeting moiety.

In some embodiments, the exogenous nucleic acid is operatively linked to an inducible or repressible promoter, e.g., to drive expression of the amino acid degradative enzyme. For instance, the promoter may be doxycycline-inducible, e.g., a P-TRE3GS promoter or active fragment or variant thereof. Examples of inducible promoters include, but are not limited, to a metallothionine-inducible promoter, a glucocorticoid-inducible promoter, a progesterone-inducible promoter, and a tetracycline-inducible promoter (which may also be doxycycline-inducible). In some embodiments, the inducer is added to culture media comprising cells that comprise the inducible promoter, e.g., at a specific stage of cell differentiation. In some embodiments, the inducer (e.g., doxycycline) is added at an amount of about 1-5, 2-4, or 3 μg/mL. In some embodiments, a repressor is withdrawn from to culture media comprising cells that comprise the repressible promoter, e.g., at a specific stage of cell differentiation. In some embodiments, the inducer is added, or the repressor is withdrawn, during maturation phase, e.g., between days 1-10, 2-9, 3-8, 4-6, or about day 5 of maturation phase. In some embodiments, the inducer is present, or the repressor is absent, between day 1, 2, 3, 4, 5, 6, 7,8, 9 or 10 of maturation and enucleation. In some embodiments, the inducer is present, or the repressor is absent, for at least 1, 2, 3, 4, 5, 6, 7, 8, 9, or 10 days. In some embodiments, the inducer is present, or the repressor is absent, from maturation day 5 to the end of differentiation. In embodiments, the inducer is present, or the repressor is absent at maturation day 9. In some embodiments, the inducer is added, or the repressor is withdrawn, when the population of erythroid cells comprises a plurality of normoblasts (e.g., basophilic, polychromatic, or orthochromatic normoblasts or a combination thereof), e.g., when 10-20%, 20-30%, 30-40%, 40-50%, 50-60%, 60-70%, or 70-80% of the cells in the population are normoblasts. In some embodiments, the inducer is added, or the repressor is withdrawn, when the population of erythroid cells comprises a plurality of pro-erythroblasts, e.g., when 10-20%, 20-30%, 30-40%, 40-50%, 50-60%, 60-70%, or 70-80% of the cells in the population are pro-erythroblasts. In some embodiments, the inducer is added, or the repressor is withdrawn, when the population of erythroid cells comprises a plurality of erythroblasts at terminal differentiation e.g., when 10-20%, 20-30%, 30-40%, 40-50%, 50-60%, 60-70%, or 70-80% of the cells in the population are erythroblasts at terminal differentiation. In some embodiments, the erythroid cell or population of erythroid cells comprises an additional exogenous protein, e.g., a transactivator, e.g., a Tet-inducible transactivator (e.g., a Tet-on-3G transactivator).

In some embodiments, the inducer is added, or the repressor is withdrawn, when the population of erythroid cells comprises one or more of (e.g., all of) endogenous GPA, band 3, or alpha4 integrin. In some embodiments, the inducer is added, or the repressor is withdrawn, during a time when about 84-100%, 85-100%, 90-100%, or 95-100% of the cells in the population are GPA-positive (e.g., when the population first reaches that level); during a time when 50-100%, 60-100%, 70-100%, 80-100%, 90-100%, 95-100%, or 98-100% of the cells in the population are band 3-positive (e.g., when the population first reaches that level); and/or during a time when about 70-100%, 80-90%, or about 85% of the cells in the population are alpha4 integrin-positive (e.g., when the population first reaches that level).

GPA, band 3, and alpha4 integrin can be detected, e.g., by a flow cytometry assay, e.g., a flow cytometry assay of Example 10 of International Application Publication No. WO2018/009838, incorporated herein by reference.

In some embodiments, the cells are produced using conjugation, e.g., sortagging or sortase-mediated conjugation, e.g., as described in International Application Publication Nos. WO2014/183071 or WO2014/183066, each of which is incorporated by reference in its entirety. In some embodiments, the cells are made by a method that does not comprise sortase-mediated conjunction.

In some embodiments, the cells are made by a method that does not comprise hypotonic loading. In some embodiments, the cells are made by a method that does not comprise a hypotonic dialysis step. In some embodiments, the cells are made by a method that does not comprise controlled cell deformation.

In some embodiments, the erythroid cells are expanded at least 1000, 2000, 5000, 10,000, 20,000, 50,000, or 100,000 fold (and optionally up to 100,000, 200,000, or 500,000 fold). The number of cells is measured, in some embodiments, using an automated cell counter. In some embodiments, the population of erythroid cells comprises at least about 30%, 35%, 40%, 45%, 50%, 55%, 60%, 65%, 70%, 75%, 80%, 85%, 90%, 95%, 96% or 98% (and optionally up to about 80, 90, or 100%) enucleated erythroid cells. In some embodiments, the population of erythroid cells comprises 70%-100%, 75%-100%, 80%-100%, 85%-100%, or 90%-100% enucleated cells. In some embodiments, the population of erythroid cells contains less than 1% live nucleated cells, e.g., contains no detectable live nucleated cells. Enucleation is measured, in some embodiments, by FACS using a nuclear stain. In some embodiments, at least about 30%, 35%, 40%, 45%, 50%, 55%, 60%, 65%, 70%, 75%, 80%, 85%, 90%, 95%, 96% or 98% (and optionally up to about 70, 80, 90, or 100%) of erythroid cells in the population comprise one or more (e.g., 2, 3, 4 or more) of the exogenous polypeptides. Level of the polypeptides is measured, in some embodiments, by erythroid cells using labeled antibodies against the polypeptides. In some embodiments, at least about 30%, 35%, 40%, 45%, 50%, 55%, 60%, 65%, 70%, 75%, 80%, 85%, 90%, 95%, 96% or 98% (and optionally up to about 70, 80, 90, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

### Physical Characteristics of Engineered Erythroid Cells

In some embodiments, the engineered erythroid cells (e.g., engineered enucleated erythroid cells) or enucleated cells (e.g., modified enucleated cells) described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments, an engineered erythroid cell, (e.g., an engineered enucleated erythroid cell) or an enucleated cell (e.g., modified enucleated cell), that includes an exogenous polypeptide described herein has physical characteristics that resemble a wild-type, untreated erythroid cell or enucleated cell. In contrast, a hypotonically-loaded erythroid cell may sometimes display aberrant physical characteristics such as increased osmotic fragility, altered cell size, reduced hemoglobin concentration, or increased phosphatidylserine levels on the outer leaflet of the cell membrane.

Osmotic fragility in Some Embodiments, the Engineered Erythroid Cell or Enucleated Cell Exhibits substantially the same osmotic membrane fragility as an isolated, uncultured erythroid cell that does not comprise an exogenous polypeptide. In some embodiments, the population of engineered erythroid cells or enucleated cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

Cell Size

In some embodiments, the engineered erythroid cell (e.g., engineered enucleated erythroid cell) or enucleated cell (e.g., modified enucleated cell) has approximately the diameter or volume as a wild-type, untreated enucleated erythroid cell.

In some embodiments, the population of engineered erythroid cells (e.g., engineered enucleated erythroid cells) or enucleated cells (e.g., modified enucleated cells) has an average diameter of about 4, 5, 6, 7, or 8 microns, and optionally the standard deviation of the population is less than 1, 2, or 3 microns. In some embodiments, the one or more engineered erythroid cell (e.g., engineered enucleated erythroid cell) or enucleated cell (e.g., modified enucleated cell) has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In some embodiments, the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In some embodiments, the mean corpuscular volume of the erythroid cells is between 80-100, 100-200, 200-300, 300-400, or 400-500 femtoliters (fL). In some embodiments, a population of engineered erythroid cells (e.g., engineered enucleated erythroid cells) or enucleated cells (e.g., modified enucleated cells) has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the engineered erythroid cell (e.g., engineered enucleated erythroid cell) or enucleated cell (e.g., modified enucleated cell) has a hemoglobin content similar to a wild-type, untreated enucleated erythroid cell or enucleated cell. In some embodiments, the engineered erythroid cells (e.g., engineered enucleated erythroid cells) or enucleated cells (e.g., modified enucleated cells) comprise at least about 20, 22, 24, 26, 28, or 30 pg, and optionally up to about 30 pg, of total hemoglobin. Hemoglobin levels are determined, in some embodiments, using the Drabkin's reagent method of Example 33 of International Application Publication No. WO2015/073587, which is herein incorporated by reference in its entirety.

Phosphatidylserine Content

In some embodiments, the engineered erythroid cells (e.g., engineered enucleated erythroid cells) or enucleated cells (e.g., modified enucleated cells), has approximately the same phosphatidylserine content on the outer leaflet of its cell membrane as a wild-type, untreated erythroid cell or enucleated cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of engineered erythroid cells (e.g., engineered enucleated erythroid cells) or enucleated cells (e.g., modified enucleated cells) comprises less than about 30, 25, 20, 15, 10, 9, 8, 6, 5, 4, 3, 2, or 1% of cells that are positive for annexin V staining. Phosphatidylserine exposure is assessed, in some embodiments, by staining for annexin-V-FITC, which binds preferentially to PS, and measuring FITC fluorescence by flow cytometry, e.g., using the method of Example 54 of International Application Publication Nos. WO2015/073587, which is herein incorporated by reference in its entirety.

Other Characteristics

In some embodiments, an engineered erythroid cell (e.g., engineered enucleated erythroid cell) or an enucleated cell, or a population of engineered erythroid cells or enucleated cells comprises one or more of (e.g., all of) endogenous GPA (C235a), transferrin receptor (CD71), Band 3 (CD233), or integrin alpha4 (C49d). These proteins can be measured, e.g., as described in Example 10 of International Application Publication No. WO2018/009838, which is herein incorporated by reference in its entirety. The percentage of GPA-positive cells and Band 3-positive cells typically increases during maturation of an erythroid cell, and the percentage of integrin alpha4-positive typically remains high throughout maturation.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% GPA⁺ (i.e., CD235a⁺) cells. In some embodiments, the population of engineered enucleated erythroid cells or enucleated cells comprises between about 50% and about 100% (e.g., from about 60% and about 100%, from about 65% and about 100%, from about 70% and about 100%, from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) GPA cells. The presence of GPA is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD71⁺ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD71⁺ cells. The presence of CD71 (transferrin receptor) is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD233⁺ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD233⁺ cells. The presence of CD233 (Band 3) is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD47⁺ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD47⁺ cells. The presence of CD47 (integrin associate protein) is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% C36⁻ (CD36-negative) cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) C36⁻ (CD36-negative) cells. The presence of C36 is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% C34⁻ (CD34-negative) cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) C34⁻ (CD34-negative) cells. The presence of C34 is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235PCD47⁺/CD233⁺ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235PCD47⁺/CD233⁺ cells.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235PCD47⁺/CD233⁺/C34⁻/C36⁻ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺/C34⁻/C36⁻ cells.

In some embodiments, a population of engineered enucleated erythroid cells or enucleated cells comprising erythroid cells comprises less than about 10%, 9%, 8%, 7%, 6%, 5%, 4%, 3%, 2%, or 1% echinocytes. In some embodiments, a population of engineered enucleated erythroid cells or enucleated cells comprising comprises less than about 10%, 9%, 8%, 7%, 6%, 5%, 4%, 3%, 2%, or 1% pyrenocytes.

### Universal Donor Erythroid Cells

In some embodiments, erythroid cells or enucleated cells described herein are autologous and/or allogeneic to the subject to which the cells will be administered. For example, erythroid cells allogeneic to the subject include one or more of blood type specific erythroid cells (e.g., the cells can be of the same blood type as the subject) or one or more universal donor erythroid cells. In some embodiments, the enucleated erythroid cells described herein have reduced immunogenicity compared to a reference cell, e.g., have lowered levels of one or more blood group antigens.

Where allogeneic cells are used for transfusion, a compatible ABO blood group can be chosen to prevent an acute intravascular hemolytic transfusion reaction. The ABO blood types are defined based on the presence or absence of the blood type antigens A and B, monosaccharide carbohydrate structures that are found at the termini of oligosaccharide chains associated with glycoproteins and glycolipids on the surface of the erythrocytes (reviewed in Liu et al., Nat. Biotech. 5:454-464 (2007)). Because group O erythrocytes contain neither A nor B antigens, they can be safely transfused into recipients of any ABO blood group, e.g., group A, B, AB, or O recipients. Group O erythrocytes are considered universal and may be used in all blood transfusions. Thus, in some embodiments, an erythroid cell described herein is type O. In contrast, group A erythroid cells may be given to group A and AB recipients, group B erythroid cells may be given to group B and AB recipients, and group AB erythroid cells may be given to AB recipients.

In some instances, it may be beneficial to convert a non-group O erythroid cell to a universal blood type. Enzymatic removal of the immunodominant monosaccharides on the surface of group A and group B erythrocytes may be used to generate a population of group O-like erythroid cells (See, e.g., Liu et al., Nat. Biotech. 25:454-464 (2007)). Group B erythroid cells may be converted using an α-galactosidase from green coffee beans. Alternatively or in addition, α-N-acetylgalactosaminidase and α-galactosidase enzymatic activities from E. meningosepticum bacteria may be used to respectively remove the immunodominant A and B antigens (Liu et al., Nat. Biotech. 25:454-464 (2007)), if present on the erythroid cells. In one example, packed erythroid cells isolated as described herein, are incubated in 200 mM glycine (pH 6.8) and 3 mM NaCl in the presence of either α-N-acetylgalactosaminidase and α-galactosidase (about 300 m/ml packed erythroid cells) for 60 min at 26° C. After treatment, the erythroid cells are washed by 3-4 rinses in saline with centrifugation and ABO-typed according to standard blood banking techniques.

While the ABO blood group system is the most important in transfusion and transplantation, in some embodiments it can be useful to match other blood groups between the erythroid cells to be administered and the recipient, or to select or make erythroid cells that are universal for one or more other (e.g., minor) blood groups. A second blood group is the Rh system, wherein an individual can be Rh+ or Rh−. Thus, in some embodiments, an erythroid cell described herein is Rh−. In some embodiments, the erythroid cell is Type O and Rh−.

In some embodiments, an erythroid cell described herein is negative for one or more minor blood group antigens, e.g., Le(a−b−) (for Lewis antigen system), Fy(a−b−) (for Duffy system), Jk(a−b−) (for Kidd system), M−N− (for MNS system), K−k− (for Kell system), Lu(a−b−) (for Lutheran system), and H-antigen negative (Bombay phenotype), or any combination thereof. In some embodiments, the erythroid cell is also Type O and/or Rh−. Minor blood groups are described, e.g., in Agarwal et al “Blood group phenotype frequencies in blood donors from a tertiary care hospital in north India” Blood Res. 2013 March; 48(1): 51-54 and Mitra et al “Blood groups systems” Indian J Anaesth. 2014 September-October; 58(5): 524-528, each of which is incorporated herein by reference in its entirety.

### Isolating Erythrocytes

Mature erythrocytes may be isolated using various methods such as, for example, a cell washer, a continuous flow cell separator, density gradient separation, fluorescence-activated cell sorting (FACS), Miltenyi immunomagnetic depletion (MACS), or a combination of these methods (See, e.g., van der Berg et al., Clin. Chem. 33:1081-1082 (1987); Bar-Zvi et al., J. Biol. Chem. 262:17719-17723 (1987); Goodman et al., Exp. Biol. Med. 232:1470-1476 (2007)).

Erythrocytes may be isolated from whole blood by simple centrifugation (See, e.g., van der Berg et al., Clin. Chem. 33:1081-1082 (1987)). For example, EDTA-anticoagulated whole blood may be centrifuged at 800×g for 10 min at 4° C. The platelet-rich plasma and buffy coat are removed and the red blood cells are washed three times with isotonic saline solution (NaCl, 9 g/L).

Alternatively, erythrocytes may be isolated using density gradient centrifugation with various separation mediums such as, for example, Ficoll, Hypaque, Histopaque, Percoll, Sigmacell, or combinations thereof. For example, a volume of Histopaque-1077 is layered on top of an equal volume of Histopaque-1119. EDTA-anticoagulated whole blood diluted 1:1 in an equal volume of isotonic saline solution (NaCl, 9 g/L) is layered on top of the Histopaque and the sample is centrifuged at 700×g for 30 min at room temperature. Under these conditions, granulocytes migrate to the 1077/1119 interface, lymphocytes, other mononuclear cells and platelets remain at the plasma/1077 interface, and the red blood cells are pelleted. The red blood cells are washed twice with isotonic saline solution.

Alternatively, erythrocytes may be isolated by centrifugation using a Percoll step gradient (See, e.g., Bar-Zvi et al., J. Biol. Chem. 262:17719-17723 (1987)). For example, fresh blood is mixed with an anticoagulant solution containing 75 mM sodium citrate and 38 mM citric acid and the cells washed briefly in Hepes-buffered saline. Leukocytes and platelets are removed by adsorption with a mixture of α-cellulose and Sigmacell (1:1). The erythrocytes are further isolated from reticulocytes and residual white blood cells by centrifugation through a 45/75% Percoll step gradient for 10 min at 2500 rpm in a Sorvall SS34 rotor. The erythrocytes are recovered in the pellet while reticulocytes band at the 45/75% interface and the remaining white blood cells band at the 0/45% interface. The Percoll is removed from the erythrocytes by several washes in Hepes-buffered saline. Other materials that may be used to generate density gradients for isolation of erythrocytes include OPTIPREP, a 60% solution of iodixanol in water (from Axis-Shield, Dundee, Scotland).

Erythrocytes may be separated from reticulocytes, for example, using flow cytometry (See, e.g., Goodman et al., Exp. Biol. Med. 232:1470-1476 (2007)). In this instance, whole blood is centrifuged (550×g, 20 min, 25° C.) to separate cells from plasma. The cell pellet is resuspended in phosphate buffered saline solution and further fractionated on Ficoll-Paque (1.077 density), for example, by centrifugation (400×g, 30 min, 25° C.) to separate the erythrocytes from the white blood cells. The resulting cell pellet is resuspended in RPMI supplemented with 10% fetal bovine serum and sorted on a FACS instrument such as, for example, a Becton Dickinson FACSCalibur (BD Biosciences, Franklin Lakes, N.J., USA) based on size and granularity.

Erythrocytes may be isolated by immunomagnetic depletion (See, e.g., Goodman, et al., (2007) Exp. Biol. Med. 232:1470-1476). In this instance, magnetic beads with cell-type specific antibodies are used to eliminate non-erythrocytes. For example, erythrocytes are isolated from the majority of other blood components using a density gradient as described herein followed by immunomagnetic depletion of any residual reticulocytes. The cells are pre-treated with human antibody serum for 20 min at 25° C. and then treated with antibodies against reticulocyte specific antigens such as, for example, CD71 and CD36. The antibodies may be directly attached to magnetic beads or conjugated to PE, for example, to which magnetic beads with anti-PE antibody will react. The antibody-magnetic bead complex is able to selectively extract residual reticulocytes, for example, from the erythrocyte population.

Erythrocytes may also be isolated using apheresis. The process of apheresis involves removal of whole blood from a subject or donor, separation of blood components using centrifugation or cell sorting, withdrawal of one or more of the separated portions, and transfusion of remaining components back into the subject or donor. A number of instruments are currently in use for this purpose such as for example the Amicus and Alyx instruments from Baxter (Deerfield, Ill., USA), the Trima Accel instrument from Gambro BCT (Lakewood, Colo., USA), and the MCS+9000 instrument from Haemonetics (Braintree, Mass., USA). Additional purification methods may be necessary to achieve the appropriate degree of cell purity.

Reticulocytes are immature red blood cells and compose approximately 1% of the red blood cells in the human body. Reticulocytes develop and mature in the bone marrow. Once released into circulation, reticulocytes rapidly undergo terminal differentiation to mature erythrocytes. Like mature erythrocytes, reticulocytes do not have a cell nucleus. Unlike mature erythrocytes, reticulocytes maintain the ability to perform protein synthesis. In some embodiments, the engineered erythroid cell comprises an enucleated reticulocyte.

Reticulocytes of varying age may be isolated from peripheral blood based on the differences in cell density as the reticulocytes mature. Reticulocytes may be isolated from peripheral blood using differential centrifugation through various density gradients. For example, Percoll gradients may be used to isolate reticulocytes (See, e.g., Noble et al., Blood 74:475-481 (1989)). Sterile isotonic Percoll solutions of density 1.096 and 1.058 g/ml are made by diluting Percoll (Sigma-Aldrich, Saint Louis, Mo., USA) to a final concentration of 10 mM triethanolamine, 117 mM NaCl, 5 mM glucose, and 1.5 mg/ml bovine serum albumin (BSA). These solutions have an osmolarity between 295 and 310 mOsm. Five milliliters, for example, of the first Percoll solution (density 1.096) is added to a sterile 15 ml conical centrifuge tube. Two milliliters, for example, of the second Percoll solution (density 1.058) is layered over the higher density first Percoll solution. Two to four milliliters of whole blood are layered on top of the tube. The tube is centrifuged at 250×g for 30 min in a refrigerated centrifuge with swing-out tube holders. Reticulocytes and some white cells migrate to the interface between the two Percoll layers. The cells at the interface are transferred to a new tube and washed twice with phosphate buffered saline (PBS) with 5 mM glucose, 0.03 mM sodium azide and 1 mg/ml BSA. Residual white blood cells are removed by chromatography in PBS over a size exclusion column.

Alternatively, reticulocytes may be isolated by positive selection using an immunomagnetic separation approach (See, e.g., Brun et al., Blood 76:2397-2403 (1990)). This approach takes advantage of the large number of transferrin receptors that are expressed on the surface of reticulocytes relative to erythrocytes prior to maturation. Magnetic beads coated with an antibody to the transferrin receptor may be used to selectively isolate reticulocytes from a mixed blood cell population. Antibodies to the transferrin receptor of a variety of mammalian species, including human, are available from commercial sources (e.g., Affinity BioReagents, Golden, Colo., USA; Sigma-Aldrich, Saint Louis, Mo., USA). The transferrin antibody may be directly linked to the magnetic beads. Alternatively, the transferrin antibody may be indirectly linked to the magnetic beads via a secondary antibody. For example, mouse monoclonal antibody 10D2 (Affinity BioReagents, Golden, Colo., USA) against human transferrin may be mixed with immunomagnetic beads coated with a sheep anti-mouse immunoglobulin G (Dynal/Invitrogen, Carlsbad, Calif., USA). The immunomagnetic beads are then incubated with a leukocyte-depleted red blood cell fraction. The beads and red blood cells are incubated at 22° C. with gentle mixing for 60-90 min followed by isolation of the beads with attached reticulocytes using a magnetic field. The isolated reticulocytes may be removed from the magnetic beads using, for example, DETACHaBEAD solution (from Invitrogen, Carlsbad, Calif., USA). Alternatively, reticulocytes may be isolated from in vitro growth and maturation of CD34+ hematopoietic stem cells using the methods described herein.

Terminally-differentiated enucleated erythrocytes can be separated from other cells based on their DNA content. In a non-limiting example, cells are first labeled with a vital DNA dye, such as Hoechst 33342 (Invitrogen Corp.). Hoechst 33342 is a cell-permeant nuclear counterstain that emits blue fluorescence when bound to double-stranded DNA. Undifferentiated precursor cells, macrophages or other nucleated cells in the culture are stained by Hoechst 33342, while enucleated erythrocytes are Hoechst-negative. The Hoechst-positive cells can be separated from enucleated erythrocytes by using fluorescence activated cell sorters or other cell sorting techniques. The Hoechst dye can be removed from the isolated erythrocytes by dialysis or other suitable methods.

### Vehicles for Polypeptides Described Herein

While in many embodiments herein, the one or more (e.g., two or more) exogenous polypeptides are situated on or in an erythroid cell (e.g., engineered enucleated erythroid cell) or enucleated cell (e.g., modified enucleated cell), it is understood that any polypeptide or combination of exogenous polypeptides described herein can also be situated on or in another vehicle. The vehicle can comprise, e.g., a cell, an erythroid cell, a corpuscle, a nanoparticle, a micelle, a liposome, or an exosome. For instance, in some aspects, the present disclosure provides a vehicle (e.g., a cell, an erythroid cell, a corpuscle, a nanoparticle, a micelle, a liposome, or an exosome) comprising, e.g., on its surface, one or more agents described herein. In some embodiments, the one or more agents comprise an agent selected from the exogenous polypeptides described herein, or a fragment or variant thereof. In some embodiments, the vehicle comprises two or more agents described herein, e.g., any pair of agents described herein.

In some embodiments, the vehicle comprises an engineered erythroid cell (e.g. an engineered enucleated erythroid cell) or an enucleated cell.

### Heterogeneous Populations of Cells

While in many embodiments herein, the one or more (e.g., two or more) exogenous polypeptides are situated on or in a single cell, it is understood that any polypeptide or combination of polypeptides described herein can also be situated on a plurality of cells. For instance, in some aspects, the disclosure provides a plurality of erythroid cells or enucleated cells, wherein a first cell of the plurality comprises a first exogenous polypeptide (e.g., comprising a first exogenous antigenic polypeptide, exogenous antigen-presenting polypeptide, exogenous costimulatory polypeptide, exogenous coinhibitory polypeptide, cytokine, and exogenous Treg costimulatory polypeptide, or a combination thereof) and a second cell of the plurality comprises a second exogenous polypeptide (e.g., comprising a second exogenous antigenic polypeptide, exogenous antigen-presenting polypeptide, exogenous costimulatory polypeptide, exogenous coinhibitory polypeptide, cytokine, and exogenous Treg costimulatory polypeptide, or a combination thereof). In some embodiments, the plurality of cells comprises two or more polypeptides described herein, e.g., any pair of polypeptides described herein. In some embodiments, less than 90%, 80%, 70%, 60%, 50%, 40%, 30%, 20%, 10%, 5%, 2%, or 1% of the cells in the plurality comprise both the first exogenous polypeptide and the second exogenous polypeptide.

### Cells Encapsulated in a Membrane

In some embodiments, engineered erythroid cells (e.g. engineered enucleated erythroid cells) or enucleated cells (e.g., modified enucleated cells), or other vehicles described herein are encapsulated in a membrane, e.g., semi-permeable membrane. In some embodiments, the membrane comprises a polysaccharide, e.g., an anionic polysaccharide alginate. In some embodiments, the semipermeable membrane does not allow cells to pass through, but allows passage of small molecules or macromolecules, e.g., metabolites, proteins, or DNA. In some embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety.

### Erythrocyte Precursor Cells

Provided herein are engineered erythrocyte precursor cells, and methods of making the engineered erythrocyte precursor cells.

Pluripotent stem cells give rise to erythrocytes by the process of erythropoiesis. The stem cell looks like a small lymphocyte and lacks the functional capabilities of the erythrocyte. The stem cells have the capacity of infinite division, something the mature cells lack. Some of the daughter cells arising from the stem cell acquire erythroid characters over generations and time. Most of the erythroid cells in the bone marrow have a distinct morphology but commitment to erythroid maturation is seen even in cells that have not acquired morphological features distinctive of the erythroid lineage. These cells are recognized by the type of colonies they form in vitro. Two such cells are recognized. Burst-forming unit erythroid (BFU-E) arise from the stem cell and gives rise to colony-forming unit erythroid (CFU-E). CFU-E gives rise to pronormoblast, the most immature of erythroid cells with a distinct morphology. BFU-E and CFU-E form a very small fraction of bone marrow cells. Morphologically five erythroid precursors are identifiable in the bone marrow stained with Romanovsky stains. The five stages from the most immature to the most mature are the proerythroblast, the basophilic normoblast (early erythroblast), polychromatophilic normoblast (intermediate erythroblast), orthochromatophilic normoblast (late erythroblast) and reticulocyte. BFU-E (burst forming unit-erythroid), CFU-E (erythroid colony-forming unit), pronormoblast (proerythroblast), basophilic normoblast, polychromatophilic normoblast and orthochromatophilic normoblast are lineage restricted.

Table 7 below summarizes the morphological features of erythrocyte precursor cells.

TABLE 7

| Cell | Nucleus |
|---|---|
| Hematopoietic stem cell (HSC) | Yes |
| CMP (Common myeloid progenitor) | Yes |
| CFU-S (spleen colony forming cell; | Yes; Can differentiate into |
| myeloid precursor cell) | erythrocytes, platelets, |
| | macrophages. |
| BFU-E (burst forming unit-erythroid) | Yes |
| CFU-E (erythroid colony-forming | Yes |
| unit) | |
| Pronormoblast (proerythroblast) | Yes; fine chromatin, many nucleoli |
| Basophilic Normoblast | Yes; granular chromatin, no |
| | nucleoli |
| Polychromatophilic Normoblast | Yes; chromatin is visibly clumped |
| | with dark staining areas |
| Orthochromatophilic normoblast | Yes; featureless nucleus with dense |
| | chromatin |

Normal human erythrocytes express CD36, an adhesion molecule of monocytes, platelets, and endothelial cells (van Schravendijk M R et al., Blood. 1992 Oct. 15; 80(8):2105-14). Accordingly, in some embodiments, an anti-CD36 antibody can be used to identify human erythrocytes.

Any type of cell known in the art that is capable of differentiating into an erythrocyte, i.e., any erythrocyte precursor cell, can be modified in accordance with the methods described herein to produce engineered erythrocyte precursor cells. In certain embodiments, the erythrocyte precursor cells modified in accordance with the methods described herein are cells that are in the process of differentiating into an erythrocyte, i.e., the cells are of a type known to exist during mammalian erythropoiesis. For example, the cells may be pluripotent hematopoietic stem cells (HSCs) or CD34+ cells, multipotent myeloid progenitor cells, CFU-S cells, BFU-E cells, CFU-E cells, pronormoblasts (proerythroblast), basophilic normoblasts, polychromatophilic normoblasts, or orthochromatophilic normoblasts. The modified erythrocyte precursor cells provided herein can be differentiated into engineered enucleated erythroid cells (e.g., reticulocytes or erythrocytes) in vitro using methods known in the art, i.e., using molecules known to promote erythropoiesis, e.g., SCF, Erythropoietin, IL-3, and/or GM-CSF, described herein below. Alternatively, the modified erythrocyte precursor cells are provided in a composition as described herein, and are capable of differentiating into erythrocytes upon administration to a subject in vivo.

### Culturing

Sources for generating engineered erythroid cells described herein include circulating erythroid cells. A suitable cell source may be isolated from a subject as described herein from subject-derived hematopoietic or erythroid progenitor cells, derived from immortalized erythroid cell lines, or derived from induced pluripotent stem cells, optionally cultured and differentiated. Methods for generating erythrocytes using cell culture techniques are well known in the art, e.g., Giarratana et al., Blood 2011, 118:5071, Huang et al. (2014) Mol. Ther. 22(2): 451-63, or Kurita et al., PLOS One 2013, 8:e59890. Protocols vary according to growth factors, starting cell lines, culture period, and morphological traits by which the resulting cells are characterized. Culture systems have also been established for blood production that may substitute for donor transfusions (Fibach et al. 1989 Blood 73:100). Recently, CD34+ cells were differentiated to the reticulocyte stage, followed by successful transfusion into a human subject (Giarratana et al., Blood 2011, 118:5071).

Provided herein are culturing methods for erythroid cells and engineered erythroid cells. Erythroid cells can be cultured from hematopoietic progenitor cells, including, for example, CD34+ hematopoietic progenitor cells (Giarratana et al., Blood 2011, 118:5071), induced pluripotent stem cells (Kurita et al., PLOS One 2013, 8:e59890), and embryonic stem cells (Hirose et al. 2013 Stem Cell Reports 1:499). Cocktails of growth and differentiation factors that are suitable to expand and differentiate progenitor cells are known in the art. Examples of suitable expansion and differentiation factors include, but are not limited to, stem cell factor (SCF), an interleukin (IL) such as IL-1, IL-2, IL-3, IL-4, IL-5, IL-6, IL-7, IL-8, IL-9, IL-11, IL-12, CSF, G-CSF, thrombopoietin (TPO), GM-CSF, erythropoietin (EPO), Flt3, Flt2, PIXY 321, and leukemia inhibitory factor (LIF).

Erythroid cells can be cultured from hematopoietic progenitors, such as CD34⁺ cells, by contacting the progenitor cells with defined factors in a multi-step culture process. For example, in some embodiments, erythroid cells can be cultured from hematopoietic progenitors in a three-step process, outlined below.

The first step may comprise contacting the cells in culture with stem cell factor (SCF) at 1-1000 ng/mL, erythropoietin (EPO) at 1-100 U/mL, and interleukin-3 (IL-3) at 0.1-100 ng/mL. The first step optionally comprises contacting the cells in culture with a ligand that binds and activates a nuclear hormone receptor, such as e.g., the glucocorticoid receptor, the estrogen receptor, the progesterone receptor, the androgen receptor, or the pregnane×receptor. The ligands for these receptors include, for example, a corticosteroid, such as, e.g., dexamethasone at 10 nM-100 μM or hydrocortisone at 10 nM-100 μM; an estrogen, such as, e.g., beta-estradiol at 10 nM-100 μM; a progestogen, such as, e.g., progesterone at 10 nM-100 μM, hydroxyprogesterone at 10 nM-100 μM, 5a-dihydroprogesterone at 10 nM-100 μM, 11-deoxycorticosterone at 10 nM-100 μM, or a synthetic progestin, such as, e.g., chlormadinone acetate at 10 nM-100 μM; an androgen, such as, e.g., testosterone at 10 nM-100 μM, dihydrotestosterone at 10 nM-100 μM or androstenedione at 10 nM-100 μM; or a pregnane×receptor ligand, such as, e.g., rifampicin at 10 nM-100 μM, hyperforin at 10 nM-100 St. John's Wort (hypericin) at 10 nM-100 μM, or vitamin E-like molecules, such as, e.g., tocopherol at 10 nM-100 The first step may also optionally comprise contacting the cells in culture with an insulin-like molecule, such as, e.g., insulin at 1-50 μ·g/mL, insulin-like growth factor 1 (IGF-1) at 1-50 μg/mL, insulin-like growth factor 2 (IGF-2) at 1-50 μg/mL, or mechano-growth factor at 1-50 μg/mL. The first step further may optionally comprise contacting the cells in culture with transferrin at 0.1-5 mg/mL.

The first step may optionally comprise contacting the cells in culture with one or more interleukins (IL) or growth factors such as, e.g., IL-1, IL-2, IL-4, IL-5, IL-6, IL-7, IL-8, IL-9, IL-11, IL-12, granulocyte colony-stimulating factor (G-CSF), macrophage colony-stimulating factor (M-CSF), granulocyte-macrophage colony-stimulating factor (GM-CSF), thrombopoietin, fibroblast growth factor (FGF), platelet-derived growth factor (PDGF), transforming growth factor beta (TGF-B), tumor necrosis factor alpha (TNF-A), megakaryocyte growth and development factor (MGDF), leukemia inhibitory factor (LIF), and Flt3 ligand. Each interleukin or growth factor may typically be supplied at a concentration of 0.1-100 ng/mL. The first step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The second step may comprise contacting the cells in culture with stem cell factor (SCF) at 1-1000 ng/mL and erythropoietin (EPO) at 1-100 U/mL. The second step may also optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The second step may further optionally comprise contacting the cells in culture with transferrin at 0.1-5 mg/mL. The second may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The third step may comprise contacting the cells in culture with erythropoietin (EPO) at 1-100 U/mL. The third step may optionally comprise contacting the cells in culture with stem cell factor (SCF) at 1-1000 ng/mL. The third step may further optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The third step may also optionally comprise contacting the cells in culture with transferrin at 0.1-5 mg/mL. The third step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

In some embodiments, methods of expansion and differentiation of the engineered erythroid cells presenting one or more exogenous polypeptides, do not include culturing the engineered erythroid cells in a medium comprising a myeloproliferative receptor (mpl) ligand.

The culture process may optionally comprise contacting cells by a method known in the art with a molecule, e.g., a DNA molecule, an RNA molecule, a mRNA, an siRNA, a microRNA, a lncRNA, a shRNA, a hormone, or a small molecule, that activates or knocks down one or more genes. Target genes can include, for example, genes that encode a transcription factor, a growth factor, or a growth factor receptor, including but not limited to, e.g., GATA1, GATA2, CMyc, hTERT, p53, EPO, SCF, insulin, EPO—R, SCF-R, transferrin-R, insulin-R.

In some embodiments, CD34+ cells are placed in a culture containing varying amounts of IMDM, FBS, glutamine, BSA, holotransferrin, insulin, dexamethasone, .beta.-estradiol, IL-3, SCF, and erythropoietin, in three separate differentiation stages for a total of 22 days.

In some embodiments, CD34+ cells are placed in a culture containing varying amounts of IMDM, FBS, glutamine, BSA, holotransferrin, insulin, dexamethasone, f3-estradiol, IL-3, SCF, and thrombopoietin, in three separate differentiation stages for a total of 14 days.

In some embodiments, CD34+ cells are placed in a culture containing varying amounts of IMDM, FBS, glutamine, BSA, holotransferrin, insulin, dexamethasone, f3-estradiol, IL-3, SCF, and GCSF, in three separate differentiation stages for a total of 15 days.

In some embodiments, the erythroid cells are expanded at least 100, 1000, 2000, 5000, 10,000, 20,000, 50,000, or 100,000 fold (and optionally up to 100,000, 200,000, or 500,000 fold). Number of cells is measured, in some embodiments, using an automated cell counter.

In some embodiments, it may be desirable during culturing to only partially differentiate the erythroid progenitor cells, e.g., hematopoietic stem cells, in vitro, allowing further differentiation, e.g., differentiation into reticulocytes or fully mature erythrocytes, to occur upon introduction to a subject in vivo (See, e.g., Neildez-Nguyen et al., Nature Biotech. 20:467-472 (2002)). It will be understood that, in various embodiments, as described herein, maturation and/or differentiation in vitro may be arrested at any stage desired. For example, isolated CD34+hematopoietic stem cells may be expanded in vitro as described elsewhere herein, e.g., in medium containing various factors, including, for example, interleukin 3, Flt3 ligand, stem cell factor, thrombopoietin, erythropoietin, transferrin, and insulin growth factor, to reach a desired stage of differentiation. The resulting engineered erythroid cells may be characterized by the surface expression of CD36 and GPA, and other characteristics specific to the particular desired cell type, and may be transfused into a subject where terminal differentiation to mature erythrocytes is allowed to occur.

In some embodiments, engineered erythroid cells are partially expanded from erythroid progenitor cells to any stage of maturation prior to but not including enucleation, and thus remain nucleated cells, e.g., erythrocyte precursor cells. In certain embodiments, the resulting cells are nucleated and erythroid lineage restricted. In certain embodiments, the resulting cells are selected from multipotent myeloid progenitor cells, CFU-S cells, BFU-E cells, CFU-E cells, pronormoblasts (proerythroblast), basophilic normoblasts, polychromatophilic normoblasts and orthochromatophilic normoblasts. The final differentiation steps, including enucleation, occur only after administration of the engineered erythroid cell to a subject, that is, in such embodiments, the enucleation step occurs in vivo. In another embodiment, engineered erythroid cells are expanded and differentiated in vitro through the stage of enucleation to become, e.g., reticulocytes. In such embodiments where the engineered erythroid cells are differentiated to the stage of reticuloyctes, the final differentiation step to become erythrocytes occurs only after administration of the engineered erythroid cell to a subject, that is, the terminal differentiation step occurs in vivo. In another embodiment, engineered erythroid cells are expanded and differentiated in vitro through the terminal differentiation stage to become erythrocytes.

It will be further recognized that in some embodiments, the engineered erythroid cells may be expanded and differentiated from erythroid progenitor cells, e.g., hematopoietic stem cells, to become hematopoietic cells of different lineage, such as, for example, to become platelets. Methods for maturing and differentiating hematopoietic cells of various lineages, such as platelets, are well known in the art to the skilled artisan. Such engineered platelets expressing exogenous polypeptides as described herein are considered to be encompassed by the present disclosure.

### Expression of Exogenous Polypeptides

In some embodiments, the engineered erythroid cells described herein are generated by contacting a suitable isolated cell, e.g., a nucleated erythroid cell, an erythroid precursor cell, or a nucleated platelet precursor cell, with an exogenous nucleic acid encoding a polypeptide of the disclosure (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides, or a combination thereof).

In some embodiments, the exogenous polypeptide is encoded by a DNA, which is contacted with a nucleated erythroid precursor cell or a nucleated platelet precursor cell. In some embodiments, the exogenous polypeptide is encoded by an RNA (e.g., an mRNA), which is contacted with a nucleated erythroid cell, an erythroid precursor cell, a nucleated platelet precursor cell.

In some embodiments, the exogenous polypeptide is contacted with a platelet, a nucleated erythroid cell, an erythroid precursor cell, a nucleated platelet precursor cell, a reticulocyte, or an erythrocyte.

In some embodiments, the exogenous polypeptide comprises an epitope tag sequence, which may be one, or a combination of, an; HA-tag, Green fluorescent protein tag, Myc-tag, chitin binding protein, maltose binding protein, glutathione-S-transferase, poly(His)tag, thioredoxin, poly(NANP), FLAG-tag, V5-tag, AviTag, Calmodulin-tag, polyglutamate-tag, E-tag, S-tag, SBP-tag, Softag-1, Softag-3, Strep-tag, TC-tag, VSV-tag, Xpress-tag, Isopeptag, SpyTag, biotin carboxyl carrier protein, Nus-tag, Fc-tag, or Ty-tag. In some embodiments, the exogenous nucleic acid encoding an exogenous polypeptide comprises the 3′ end of a gene sequence for the exogenous polypeptide that is fused to an epitope tag sequence (e.g., at the N-terminus or C-terminus), of which may be one, or a combination of: an; an HA-tag, gGreen fluorescent protein tag, Myc-tag, chitin binding protein, maltose binding protein, glutathione-S-transferase, poly(His)tag, thioredoxin, poly(NANP), FLAG-tag, V5-tag, AviTag, Calmodulin-tag, polyglutamate-tag, E-tag, S-tag, SBP-tag, Softag-1, Softag-3, Strep-tag, TC-tag, VSV-tag, Xpress-tag, Isopeptag, SpyTag, biotin carboxyl carrier protein, Nus-tag, Fc-tag, or Ty-tag. In some embodiments, the exogenous polypeptide comprises an epitope tag, such as an HA epitope tag (YPYDVPDYA (SEQ ID NO: 1)), a cMyc tag (EQKLISEEDL (SEQ ID NO:2)), or a Flag tag (DYKDDDDK (SEQ ID NO: 3)). The epitope tag may be used for the easy detection and quantification of expression using antibodies against the epitope tag by flow cytometry, western blot, or immunoprecipitation.

An exogenous polypeptide may be expressed from a transgene introduced into an erythroid cell by electroporation, chemical or polymeric transfection, viral transduction, mechanical membrane disruption, or other method; an exogenous polypeptide that is expressed from mRNA that is introduced into a cell by electroporation, chemical or polymeric transfection, viral transduction, mechanical membrane disruption, or other method; an exogenous polypeptide that is over-expressed from the native locus by the introduction of an external factor, e.g., a transcriptional activator, transcriptional repressor, or secretory pathway enhancer; and/or a polypeptide that is synthesized, extracted, or produced from a production cell or other external system and incorporated into the erythroid cell.

In certain embodiments, the introducing step comprises viral transduction. In some embodiments, the introducing step comprises electroporation. In another embodiment, the introducing step comprises utilizing one or more of liposome mediated transfer, adenovirus, adeno-associated virus, herpes virus, a retroviral based vector, lipofection, and a lentiviral vector.

In some embodiments, the introducing step comprises introducing the first exogenous nucleic acid encoding the first exogenous polypeptide by transfection of a lentiviral vector.

Exogenous nucleic acids (e.g., comprising DNA or RNA) encoding an exogenous polypeptide (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides) can be introduced by transfection of single or multiple copies of genes, transduction with a virus, or electroporation. Methods for expression of exogenous proteins in mammalian cells are well known in the art. For example, expression of exogenous factor IX in hematopoietic cells is induced by viral transduction of CD34⁺ progenitor cells, see Chang et al., Nat Biotechnol 2006, 24:1017.

In some embodiments, the DNA or RNA is codon optimized.

In some embodiments, the two or more polypeptides are encoded in a single nucleic acid, e.g. a single vector. In some embodiments, the single vector has a separate promoter for each gene, has two proteins that are initially transcribed into a single polypeptide having a protease cleavage site in the middle, so that subsequent proteolytic processing yields two proteins, or any other suitable configuration. In some embodiments, when there are more than one polypeptides (e.g. two or more) the polypeptides may be encoded in a single nucleic acid, e.g. a single vector. When exogenous antigenic polypeptides, exogenous antigen-presenting polypeptides, exogenous costimulatory polypeptides, exogenous coinhibitory polypeptides, cytokines, and/or exogenous Treg costimulatory polypeptides are encoded by the same exogenous nucleic acid (e.g., a vector), there are multiple possible sub-strategies useful for co-expression of the polypeptides. In some embodiments, the single exogenous nucleic acid (e.g., vector) has a separate promoter for each gene encoding an exogenous nucleic acid. In some embodiments, the exogenous nucleic acid encodes the two (or more) exogenous polypeptides whereby a “self-cleaving” 2A element is disposed between the cistrons encoding the exogenous polypeptides. The 2A element is believed to function by making the ribosome skip the synthesis of a peptide bond at the C-terminus of a 2A element, leading to separation between the end of the 2A sequence and the next polypeptide downstream (see, e.g., Holst et al. (2008) Nat. Immunol. 6:658-66). In some embodiments, the recombinant nucleic acid comprises a gene encoding a first exogenous polypeptide, wherein the first exogenous polypeptide is an antigen-presenting polypeptide, or a variant thereof, and a gene encoding a second exogenous polypeptide, wherein the second exogenous polypeptide is an exogenous antigenic polypeptide, or a variant thereof, wherein the second gene is separated from the gene encoding the first exogenous polypeptide by a viral-derived 2A element (gagggcagaggaagtcttctaacatgcggtgacgtggaggsgsstcccggccct (SEQ ID NO: 4). Multiple 2A elements are known in the art and can be used as described herein including T2A, P2A, E2A, and F2A (see, e.g., Liu et al. (2017) Sci. Rep. 7(1): 2193.

For dual expression via two promoters, the MSCV promoter may be used as a first promoter and the EF1 promoter as a second promoter, although the disclosure is not to be limited by these two exemplary promoters. Another strategy is to express both two or more exogenous polypeptides by inserting an internal ribosome entry site (IRES) between the two genes encoding the polypeptides. Still another strategy is to express two or more exogenous polypeptides as direct peptide fusions separated by a linker.

In some embodiments, the two or more polypeptides are encoded by two or more exogenous nucleic acids, e.g., each vector encodes one of the exogenous polypeptides.

For dual expression via two promoters, the MSCV promoter may be used as a first promoter and the EF1 promoter as a second promoter, although the disclosure is not to be limited by these two exemplary promoters. Another strategy is to express both two or more exogenous polypeptides by inserting an internal ribosome entry site (IRES) between the two genes encoding the polypeptides. Still another strategy is to express two or more exogenous polypeptides as direct peptide fusions separated by a linker.

In some embodiments, the two or more polypeptides are encoded by two or more exogenous nucleic acids, e.g., each vector encodes one of the exogenous polypeptides.

In certain embodiments, the lentiviral vector is used which comprises a promoter selected from the group consisting of beta-globin promoter, murine stem cell virus (MSCV) promoter, Gibbon ape leukemia virus (GALV) promoter, human elongation factor 1alpha (EF1alpha) promoter, CAG CMV immediate early enhancer and the chicken beta-actin (CAG), and human phosphoglycerate kinase 1 (PGK) promoter.

In some embodiments, the two or more polypeptides are encoded in two or more nucleic acids, e.g., each vector encodes one of the polypeptides.

Nucleic acids such as DNA expression vectors or mRNA for producing the exogenous polypeptides may be introduced into progenitor cells (e.g., an erythroid cell progenitor or a platelet progenitor and the like) that are suitable to produce the exogenous polypeptides described herein. The progenitor cells can be isolated from an original source or obtained from expanded progenitor cell population via routine recombinant technology as provided herein. In some instances, the expression vectors can be designed such that they can incorporate into the genome of cells by homologous or non-homologous recombination by methods known in the art.

In some embodiments, hematopoietic progenitor cells, e.g., CD34+hematopoietic stem cells, are contacted with a nucleic acid or nucleic acids encoding one or more exogenous polypeptides, and the cells are allowed to expand and differentiate in culture.

In some instances, e.g., for an aAPC that is an erythroid cell comprising one or more exogenous polypeptides (e.g. exogenous antigenic polypeptides, exogenous antigen-presenting polypeptides, exogenous costimulatory polypeptides, exogenous coinhibitory polypeptides, exogenous regulatory T cell expansion polypeptides, cytokines, and exogenous placeholder polypeptides), a nucleic acid encoding a polypeptide that can selectively target and cut the genome, for example a CRISPR/Cas9, transcriptional activator-like effector nuclease (TALEN), or zinc finger nuclease, is used to direct the insertion of the exogenous nucleic acid of the expression vector encoding the exogenous polypeptide to a particular genomic location, for example the CR1 locus (1q32.2), the hemoglobin locus (11p15.4). Thus, in one aspect, the present disclosure features a method of making an immunologically compatible artificial antigen presenting cell (aAPC), wherein the aAPC comprises an engineered erythroid cell that includes an exogenous antigenic polypeptide, the method comprising contacting the nucleated erythroid cell, an erythroid precursor cell, or a nucleated platelet precursor cell with a nuclease and at least one gRNA which cleaves an endogenous HLA-E or HLA-G nucleic acid, wherein the endogenous HLA-E or HLA-G nucleic acid is repaired by a gene editing pathway and results in a decrease in the level of expression of the endogenous HLA-E or HLA-G nucleic acid.

In some embodiments, one or more exogenous polypeptides (e.g. exogenous antigenic polypeptides, exogenous antigen-presenting polypeptides, exogenous costimulatory polypeptides, exogenous coinhibitory polypeptides, cytokines, and exogenous Treg costimulatory polypeptides) may be cloned into plasmid constructs for transfection. Methods for transferring expression vectors into cells that are suitable to produce the aAPCs described herein include, but are not limited to, viral mediated gene transfer, liposome mediated transfer, transformation, gene guns, transfection and transduction, e.g., viral mediated gene transfer such as the use of vectors based on DNA viruses such as adenovirus, adenoassociated virus and herpes virus, as well as retroviral based vectors. Examples of modes of gene transfer include e.g., naked DNA, CaPO₄ precipitation, DEAE dextran, electroporation, protoplast fusion, lipofection, and cell microinjection.

In some embodiments, recombinant DNA encoding each exogenous polypeptide may be cloned into a lentiviral vector plasmid for integration into erythroid cells. In some embodiments, the lentiviral vector comprises DNA encoding a single exogenous polypeptide for integration into erythroid cells. In other embodiments, the lentiviral vector comprises two, three, four or more exogenous polypeptides as described herein for integration into erythroid cells. In some embodiments, recombinant DNA encoding the one or more exogenous polypeptides may be cloned into a plasmid DNA construct encoding a selectable trait, such as an antibiotic resistance gene. In some embodiments, recombinant DNA encoding the exogenous polypeptides may be cloned into a plasmid construct that is adapted to stably express each recombinant protein in the erythroid cells.

In some embodiments, the lentiviral system may be employed where the transfer vector with exogenous polypeptides sequences (e.g., one, two, three, four or more exogenous polypeptide sequences), an envelope vector, and/or one or more packaging vectors are each transfected into host cells for virus production. In some embodiments, the lentiviral vectors may be transfected into host cells by any of calcium phosphate precipitation transfection, lipid-based transfection, or electroporation, and incubated overnight. For embodiments where the exogenous polypeptide sequence may be accompanied by a fluorescence reporter, inspection of the host cells for florescence may be checked after overnight incubation. The culture medium of the host cells comprising virus particles may be harvested 2 or 3 times every 8-12 hours and centrifuged to sediment detached cells and debris. The culture medium may then be used directly, frozen or concentrated as needed.

A progenitor cell subject to transfer of an exogenous nucleic acid that encodes an exogenous polypeptide can be cultured under suitable conditions allowing for differentiation and enucleation, e.g., the in vitro culturing process described herein.

Isolated erythroid precursor cells (e.g., a CD34⁺ hematopoietic stem cells) may be transfected with mRNA encoding one or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides) to generate an aAPC. Messenger RNA may be derived from in vitro transcription of a cDNA plasmid construct containing the coding sequence corresponding to the one or more exogenous polypeptides. For example, the cDNA sequence corresponding to the exogenous polypeptide may be inserted into a cloning vector containing a promoter sequence compatible with specific RNA polymerases. For example, the cloning vector ZAP EXPRESS pBK-CMV (Stratagene, La Jolla, Calif., USA) contains T3 and T7 promoter sequence compatible with T3 and T7 RNA polymerase, respectively. For in vitro transcription of sense mRNA, the plasmid is linearized at a restriction site downstream of the stop codon(s) corresponding to the end of the coding sequence of the exogenous polypeptide. The mRNA is transcribed from the linear DNA template using a commercially available kit such as, for example, the RNAMAXX High Yield Transcription Kit (from Stratagene, La Jolla, Calif., USA). In some instances, it may be desirable to generate 5′-m7GpppG-capped mRNA. As such, transcription of a linearized cDNA template may be carried out using, for example, the mMESSAGE mMACHINE High Yield Capped RNA Transcription Kit from Ambion (Austin, Tex., USA). Transcription may be carried out in a reaction volume of 20-100 μl at 37° C. for 30 min to 4 h. The transcribed mRNA is purified from the reaction mix by a brief treatment with DNase I to eliminate the linearized DNA template followed by precipitation in 70% ethanol in the presence of lithium chloride, sodium acetate or ammonium acetate. The integrity of the transcribed mRNA may be assessed using electrophoresis with an agarose-formaldehyde gel or commercially available Novex pre-cast TBE gels (e.g., Novex, Invitrogen, Carlsbad, Calif., USA).

Messenger RNA encoding the one or more exogenous polypeptides (e.g., exogenous antigenic polypeptides, exogenous antigen-presenting polypeptides, exogenous costimulatory polypeptides, exogenous coinhibitory polypeptides, cytokines and exogenous Treg costimulatory polypeptides) may be introduced into erythroid precursor cells (e.g., a CD34⁺ hematopoietic stem cell) using a variety of approaches including, for example, lipofection and electroporation (van Tandeloo et al., Blood 98:49-56 (2001)). For lipofection, for example, 5 μg of in vitro transcribed mRNA in Opti-MEM (Invitrogen, Carlsbad, Calif., USA) is incubated for 5-15 min at a 1:4 ratio with the cationic lipid DMRIE-C (Invitrogen). Alternatively, a variety of other cationic lipids or cationic polymers may be used to transfect cells with mRNA including, for example, DOTAP, various forms of polyethylenimine, and polyL-lysine (Sigma-Aldrich, Saint Louis, Mo., USA), and Superfect (Qiagen, Inc., Valencia, Calif., USA; See, e.g., Bettinger et al., Nucleic Acids Res. 29:3882-3891 (2001)). The resulting mRNA/lipid complexes are incubated with cells (1-2×10⁶ cells/ml) for 2 h at 37° C., washed and returned to culture. For electroporation, for example, about 5 to 20×10⁶ cells in 500 μl of Opti-MEM (Invitrogen, Carlsbad, Calif., USA) are mixed with about 20 μg of in vitro transcribed mRNA and electroporated in a 0.4-cm cuvette using, for example, and Easyject Plus device (EquiBio, Kent, United Kingdom). In some instances, it may be necessary to test various voltages, capacitances and electroporation volumes to determine the useful conditions for transfection of a particular mRNA into a. In general, the electroporation parameters required to efficiently transfect cells with mRNA appear to be less detrimental to cells than those required for electroporation of DNA (van Tandeloo et al., Blood 98:49-56 (2001)).

Alternatively, mRNA may be transfected into an erythroid precursor cells (e.g., a CD34⁺ cell) using a peptide-mediated RNA delivery strategy (see, e.g., Bettinger et al., Nucleic Acids Res. 29:3882-3891 (2001)). For example, the cationic lipid polyethylenimine 2 kDA (Sigma-Aldrich, Saint Louis, Mo., USA) may be combined with the melittin peptide (Alta Biosciences, Birmingham, UK) to increase the efficiency of mRNA transfection, particularly in post-mitotic primary cells. The mellitin peptide may be conjugated to the PEI using a disulfide cross-linker such as, for example, the hetero-bifunctional cross-linker succinimidyl 3-(2-pyridyldithio) propionate. In vitro transcribed mRNA is preincubated for 5 to 15 min with the mellitin-PEI to form an RNA/peptide/lipid complex. This complex is then added to cells in serum-free culture medium for 2 to 4 h at 37° C. in a 5% CO₂ humidified environment and then removed and the transfected cells allowed to continue growing in culture.

In some embodiments, the aAPC is generated by contacting a suitable isolated erythroid precursor cell or a platelet precursor cell with an exogenous nucleic acid encoding one or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides). In some embodiments, the exogenous polypeptide is encoded by a DNA, which is contacted with a nucleated erythroid precursor cell or a nucleated platelet precursor cell. In some embodiments, the exogenous polypeptide is encoded by an RNA, which is contacted with a platelet, a nucleated erythroid cell, or a nucleated platelet precursor cell.

The one or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides) may be genetically introduced into erythroid precursor cells, platelet precursor, or nucleated erythroid cells prior to terminal differentiation using a variety of DNA techniques, including transient or stable transfections and gene therapy approaches. The exogenous polypeptides may be expressed on the surface and/or in the cytoplasm of erythroid cell or platelet.

Viral gene transfer may be used to transfect the cells with DNA encoding one or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides). A number of viruses may be used as gene transfer vehicles including Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example (See, e.g., Osten et al., HEP 178:177-202 (2007)). Retroviruses, for example, efficiently transduce mammalian cells including human cells and integrate into chromosomes, conferring stable gene transfer.

One or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides) may be transfected into an erythroid precursor cell, a platelet precursor, or a nucleated erythroid cell, expressed and subsequently retained and exhibited in an engineered erythroid cell (e.g., an engineered enucleated erythroid cell) or an enucleated cell (e.g., modified enucleated cell), as described herein. A suitable vector is the Moloney murine leukemia virus (MMLV) vector backbone (Malik et al., Blood 91:2664-2671 (1998)). Vectors based on MMLV, an oncogenic retrovirus, are currently used in gene therapy clinical trials (Hossle et al., News Physiol. Sci. 17:87-92 (2002)). For example, a DNA construct containing the cDNA encoding an exogenous polypeptide can be generated in the MMLV vector backbone using standard molecular biology techniques. The construct is transfected into a packaging cell line such as, for example, PA317 cells and the viral supernatant is used to transfect producer cells such as, for example, PG13 cells. The PG13 viral supernatant is incubated with an erythroid precursor cell, a platelet precursor, or a nucleated erythroid cell that has been isolated and cultured or has been freshly isolated as described herein. The expression of the exogenous polypeptide may be monitored using FACS analysis (fluorescence-activated cell sorting), for example, with a fluorescently labeled antibody directed against the exogenous polypeptide, if it is located on the surface of the aAPC. Similar methods may be used to express an exogenous polypeptide that is located in the inside of the aAPC.

Optionally, a fluorescent tracking molecule such as, for example, green fluorescent protein (GFP) may be transfected using a viral-based approach (Tao et al., Stem Cells 25:670-678 (2007)). Ecotopic retroviral vectors containing DNA encoding the enhanced green fluorescent protein (EGFP) or a red fluorescent protein (e.g., DsRed-Express) are packaged using a packaging cell such as, for example, the Phoenix-Eco cell line (distributed by Orbigen, San Diego, Calif.). Packaging cell lines stably express viral proteins needed for proper viral packaging including, for example, gag, pol, and env. Supernatants from the Phoenix-Eco cells into which viral particles have been shed are used to transduce e.g., erythroid precursor cells, platelet precursors, or a nucleated erythroid cells. In some instances, transduction may be performed on a specially coated surface such as, for example, fragments of recombinant fibronectin to improve the efficiency of retroviral mediated gene transfer (e.g., RetroNectin, Takara Bio USA, Madison, Wis.). Cells are incubated in RetroNectin-coated plates with retroviral Phoenix-Eco supernatants plus suitable co-factors. Transduction may be repeated the next day. In this instance, the percentage of cells expressing EGFP or DsRed-Express may be assessed by FACS. Other reporter genes that may be used to assess transduction efficiency include, for example, beta-galactosidase, chloramphenicol acetyltransferase, and luciferase as well as low-affinity nerve growth factor receptor (LNGFR), and the human cell surface CD24 antigen (Bierhuizen et al., Leukemia 13:605-613 (1999)).

Nonviral vectors may be used to introduce genetic material into suitable erythroid cells, platelets or precursors thereof to generate aAPCs. Nonviral-mediated gene transfer differs from viral-mediated gene transfer in that the plasmid vectors contain no proteins, are less toxic and easier to scale up, and have no host cell preferences. The “naked DNA” of plasmid vectors is by itself inefficient in delivering genetic material encoding a polypeptide to a cell and therefore is combined with a gene delivery method that enables entry into cells. A number of delivery methods may be used to transfer nonviral vectors into suitable erythroid cells, platelets or precursors thereof including chemical and physical methods.

A nonviral vector encoding one or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides) may be introduced into suitable erythroid cells, platelets or precursors thereof using synthetic macromolecules such as cationic lipids and polymers (Papapetrou et al., Gene Therapy 12:S118-S130 (2005)). Cationic liposomes, for example form complexes with DNA through charge interactions. The positively charged DNA/lipid complexes bind to the negative cell surface and are taken up by the cell by endocytosis. This approach may be used, for example, to transfect hematopoietic cells (See, e.g., Keller et al., Gene Therapy 6:931-938 (1999)). For erythroid cells, platelets or precursors thereof the plasmid DNA (approximately 0.5 μg in 25-100 μL of a serum free medium, such as, for example, OptiMEM (Invitrogen, Carlsbad, Calif.)) is mixed with a cationic liposome (approximately 4 μ·g in 25 μ·L of serum free medium) such as the commercially available transfection reagent Lipofectamine™ (Invitrogen, Carlsbad, Calif.) and allowed to incubate for at least 20 min to form complexes. The DNA/liposome complex is added to suitable erythroid cells, platelets or precursors thereof and allowed to incubate for 5-24 h, after which time transgene expression of the polypeptide may be assayed. Alternatively, other commercially available liposome transfection agents may be used (e.g., In vivo GeneSHUTTLE, Qbiogene, Carlsbad, Calif.).

Optionally, a cationic polymer such as, for example, polyethylenimine (PEI) may be used to efficiently transfect erythroid cell progenitor cells, for example hematopoietic and umbilical cord blood-derived CD34+ cells (See, e.g., Shin et al., Biochim. Biophys. Acta 1725:377-384 (2005)). Human CD34+ cells are isolated from human umbilical cord blood and cultured in Iscove's modified Dulbecco's medium supplemented with 200 ng/ml stem cell factor and 20% heat-inactivated fetal bovine serum. Plasmid DNA encoding the exogenous polypeptide is incubated with branched or linear PEIs varying in size from 0.8 K to 750 K (Sigma Aldrich, Saint Louis, Mo., USA; Fermetas, Hanover, Md., USA). PEI is prepared as a stock solution at 4.2 mg/ml distilled water and slightly acidified to pH 5.0 using HCl. The DNA may be combined with the PEI for 30 min at room temperature at various nitrogen/phosphate ratios based on the calculation that 1 μg of DNA contains 3 nmol phosphate and 1 μl of PEI stock solution contains 10 nmol amine nitrogen. The isolated CD34+ cells are seeded with the DNA/cationic complex, centrifuged at 280×g for 5 min and incubated in culture medium for 4 or more h until gene expression of the polypeptide is assessed.

A plasmid vector may be introduced into suitable erythroid cells, platelets or precursors thereof using a physical method such as particle-mediated transfection, “gene gun”, biolistics, or particle bombardment technology (Papapetrou, et al., (2005) Gene Therapy 12:S118-S130). In this instance, DNA encoding the polypeptide is absorbed onto gold particles and administered to cells by a particle gun. This approach may be used, for example, to transfect erythroid progenitor cells, e.g., hematopoietic stem cells derived from umbilical cord blood (See, e.g., Verma et al., Gene Therapy 5:692-699 (1998)). As such, umbilical cord blood is isolated and diluted three fold in phosphate buffered saline. CD34+ cells are purified using an anti-CD34 monoclonal antibody in combination with magnetic microbeads coated with a secondary antibody and a magnetic isolation system (e.g., Miltenyi MiniMac System, Auburn, Calif., USA). The CD34+enriched cells may be cultured as described herein. For transfection, plasmid DNA encoding the polypeptide is precipitated onto a particle, for example gold beads, by treatment with calcium chloride and spermidine. Following washing of the DNA-coated beads with ethanol, the beads may be delivered into the cultured cells using, for example, a Biolistic PDS-1000/He System (Bio-Rad, Hercules, Calif., USA). A reporter gene such as, for example, beta-galactosidase, chloramphenicol acetyltransferase, luciferase, or green fluorescent protein may be used to assess efficiency of transfection.

Optionally, electroporation methods may be used to introduce a plasmid vector into suitable erythroid cells, platelets or precursors thereof. Electroporation creates transient pores in the cell membrane, allowing for the introduction of various molecules into the cells including, for example, DNA and RNA as well as antibodies and drugs. As such, CD34+ cells are isolated and cultured as described herein. Immediately prior to electroporation, the cells are isolated by centrifugation for 10 min at 250×g at room temperature and resuspended at 0.2-10×10⁶ viable cells/ml in an electroporation buffer such as, for example, X-VIVO 10 supplemented with 1.0% human serum albumin (HSA). The plasmid DNA (1-50 μg) is added to an appropriate electroporation cuvette along with 500 μl of cell suspension. Electroporation may be done using, for example, an ECM 600 electroporator (Genetronics, San Diego, Calif., USA) with voltages ranging from 200 V to 280 V and pulse lengths ranging from 25 to 70 milliseconds. A number of alternative electroporation instruments are commercially available and may be used for this purpose (e.g., Gene Pulser XCELL, BioRad, Hercules, Calif.; Cellject Duo, Thermo Science, Milford, Mass.). Alternatively, efficient electroporation of isolated CD34+ cells may be performed using the following parameters: 4 mm cuvette, 1600 μF, 550 V/cm, and 10 μg of DNA per 500 μl of cells at 1×10⁵ cells/ml (Oldak et al., Acta Biochimica Polonica 49:625-632 (2002)).

Nucleofection, a form of electroporation, may also be used to transfect suitable erythroid cells, platelets or precursors thereof. In this instance, transfection is performed using electrical parameters in cell-type specific solutions that enable DNA (or other reagents) to be directly transported to the nucleus thus reducing the risk of possible degradation in the cytoplasm. For example, a Human CD34 CELL NYCLEOFECTOR Kit (from Amaxa Inc.) may be used to transfect suitable erythroid cells, platelets or precursors thereof. In this instance, 1-5×10⁶ cells in Human CD34 Cell NUCLEOFECTOR Solution are mixed with 1-5 μg of DNA and transfected in the NUCLEOFECTOR instrument using preprogrammed settings as determined by the manufacturer.

Erythroid cells, platelets or precursors thereof may be non-virally transfected with a conventional expression vector which is unable to self-replicate in mammalian cells unless it is integrated in the genome. Alternatively, erythroid cells, platelets or precursors thereof may be transfected with an episomal vector which may persist in the host nucleus as autonomously replicating genetic units without integration into chromosomes (Papapetrou et al., Gene Therapy 12:S118-S130 (2005)). These vectors exploit genetic elements derived from viruses that are normally extrachromosomally replicating in cells upon latent infection such as, for example, EBV, human polyomavirus BK, bovine papilloma virus-1 (BPV-1), herpes simplex virus-1 (HSV) and Simian virus 40 (SV40). Mammalian artificial chromosomes may also be used for nonviral gene transfer (Vanderbyl et al., Exp. Hematol. 33:1470-1476 (2005)).

Exogenous nucleic acids encoding one or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides) may be assembled into expression vectors by standard molecular biology methods known in the art, e.g., restriction digestion, overlap-extension PCR, and Gibson assembly.

Exogenous nucleic acids may comprise a gene encoding one or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides) that are not normally expressed on the cell surface, e.g., of an erythroid cell, fused to a gene that encodes an endogenous or native membrane protein, such that the exogenous polypeptide is expressed on the cell surface. For example, a exogenous gene encoding an exogenous antigenic polypeptide can be cloned at the N terminus following the leader sequence of a type 1 membrane protein, at the C terminus of a type 2 membrane protein, or upstream of the GPI attachment site of a GPI-linked membrane protein. In some embodiments, an exogenous nucleic acid encoding a membrane-localized exogenous polypeptide (e.g., a polypeptide comprising a Type I membrane protein transmembrane domain) provided herein comprises a leader sequence (also known as a signal peptide, e.g., a β2M leader sequence or a GPA leader sequence) for targeting of the exogenous polypeptide to the membrane of a cell. In some embodiments, the leader sequence is cleaved off of the mature exogenous polypeptide by a cellular signal peptidase.

Standard cloning methods can be used to introduce flexible amino acid linkers between two fused genes. For example, the flexible linker is a poly-glycine poly-serine linker such as [Gly₄Ser]₃ (SEQ ID NO: 5) commonly used in generating single-chain antibody fragments from full-length antibodies (Antibody Engineering: Methods & Protocols, Lo 2004), or ala-gly-ser-thr polypeptides such as those used to generate single-chain Arc repressors (Robinson & Sauer, PNAS 1998). In some embodiments, the flexible linker provides the polypeptide with more flexibility and steric freedom than the equivalent construct without the flexible linker.

An epitope tag may be placed between two fused genes, such as, e.g., a nucleic acid sequence encoding an HA epitope tag—amino acids YPYDVPDYA (SEQ ID NO: 1), a CMyc tag—amino acids EQKLISEEDL (SEQ ID NO: 2), or a Flag tag—amino acids DYKDDDDK (SEQ ID NO: 3). The epitope tag may be used for the facile detection and quantification of expression using antibodies against the epitope tag by flow cytometry, western blot, or immunoprecipitation.

In some embodiments, the aAPC comprises one or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides) and at least one other heterologous polypeptide. The at least one other heterologous polypeptide can be a fluorescent protein. The fluorescent protein can be used as a reporter to assess transduction efficiency. In some embodiments, the fluorescent protein is used as a reporter to assess expression levels of the exogenous polypeptide if both are made from the same transcript. In some embodiments, the at least one other polypeptide is heterologous and provides a function, such as, e.g., multiple antigens, multiple capture targets, enzyme cascade. In some embodiments, the recombinant nucleic acid comprises a gene encoding an antigenic polypeptide and a second gene, wherein the second gene is separated from the gene encoding the antigenic polypeptide by a viral-derived T2A sequence (gagggcagaggaagtcttctaacatgcggtgacgtggaggsgsstcccggccct (SEQ ID NO: 4)) that is post-translationally cleaved into two mature proteins.

In some embodiments, the exogenous nucleic acid encoding an exogenous antigen-presenting polypeptide comprises a gene sequence for an HLA-E polypeptide or a HLA-G polypeptide that is fused to the 3′ end of the sequence for Kell and amplified using PCR. In some embodiments, the exogenous nucleic acid encoding an exogenous antigen-presenting polypeptide comprises a gene sequence for an HLA-E polypeptide or a HLA-G polypeptide that is fused to a poly-glycine/serine linker, followed by the 3′ end of the sequence for Kell, and amplified using PCR. In some embodiments, the exogenous nucleic acid encoding an exogenous antigen-presenting polypeptide comprises the 3′ end of a gene sequence for an HLA-E polypeptide or a HLA-G polypeptide that is fused to an epitope tag sequence, of which may be one, or a combination of, an; HA-tag, Green fluorescent protein tag, Myc-tag, chitin binding protein, maltose binding protein, glutathione-S-transferase, poly(His)tag, thioredoxin, poly(NANP), FLAG-tag, V5-tag, AviTag, Calmodulin-tag, polyglutamate-tag, E-tag, S-tag, SBP-tag, Softag-1, Softag-3, Strep-tag, TC-tag, VSV-tag, Xpress-tag, Isopeptag, SpyTag, biotin carboxyl carrier protein, Nus-tag, Fc-tag, or Ty-tag. The entire construct is fused to the 3′ end of the sequence for Kell and then amplified using PCR. The exogenous gene constructs encoding the various exogenous antigen-presenting polypeptides are, for example, subsequently loaded into a lentiviral vector and used to transduce a cell population.

In some embodiments, a population of erythroid cells is incubated with lentiviral vectors comprising exogenous nucleic acid encoding one or more exogenous polypeptides (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides), specific plasmids of which may include; pLKO.1 puro, PLKO.1-TRC cloning vector, pSico, FUGW, pLVTHM, pLJM1, pLion11, pMD2.G, pCMV-VSV-G, pCI-VSVG, pCMV-dR8.2 dvpr, psPAX2, pRSV-Rev, and pMDLg/pRRE to generate an aAPC. The vectors may be administered at 10, 100, 1,000, 10,000 pfu and incubated for 12 hrs.

In certain embodiments, the aAPC is an erythroid cell that presents an exogenous antigen-presenting polypeptide that is conjugated to one or more exogenous antigenic polypeptides. In other embodiments, the aAPC is an erythroid cell that includes an exogenous antigen-presenting polypeptide, an exogenous antigenic polypeptide and at least one exogenous costimulatory polypeptide that is part of a conjugate pair. In other embodiments, the aAPC is an erythroid cell that includes an exogenous antigen-presenting polypeptide, an exogenous antigenic polypeptide and at least one exogenous coinhibitory polypeptide that is part of a conjugate pair. In some embodiments, the erythroid cell is an enucleated cell. In some embodiments, the erythroid cell is a nucleated cell.

Conjugation may be achieved chemically or enzymatically. Chemical conjugation may be accomplished by covalent bonding of the exogenous antigen-presenting polypeptide to one or more exogenous antigenic polypeptides, with or without the use of a linker. Chemical conjugation may be accomplished by the covalent bonding of a costimulatory polypeptide and a binding pair member, with or without the use of a linker. Chemical conjugation may be accomplished by the covalent bonding of a coinhibitory polypeptide and a binding pair member, with or without the use of a linker. The formation of such conjugates is within the skill of artisans and various techniques are known for accomplishing the conjugation, with the choice of the particular technique being guided by the materials to be conjugated. The addition of amino acids to the polypeptide (C- or N-terminal) which contain ionizable side chains, e.g., aspartic acid, glutamic acid, lysine, arginine, cysteine, histidine, or tyrosine, and are not contained in the active portion of the polypeptide sequence, serve in their unprotonated state as a potent nucleophile to engage in various bioconjugation reactions with reactive groups attached to polymers, e.g., homo- or hetero-bi-functional PEG (e.g., Lutolf and Hubbell, Biomacromolecules 2003; 4:713-22, Hermanson, Bioconjugate Techniques, London. Academic Press Ltd; 1996).

In an embodiment, the exogenous antigen-presenting polypeptide may be bound to one or more exogenous antigenic polypeptides through a biotin-streptavidin bridge. In other embodiments, the costimulatory polypeptide and a binding pair member are bound through a biotin-streptavidin bridge. In other embodiments, the coinhibitory polypeptide and a binding pair member are bound through a biotin-streptavidin bridge.

For example, a biotinylated antigenic polypeptide may be linked to a non-specifically biotinylated surface of the exogenous antigen-presenting polypeptide through a streptavidin bridge. Biotin conjugation can occur by a number of chemical means (See, e.g., Hirsch et al., Methods Mol. Biol. 295: 135-154 (2004)). The exogenous antigen-presenting polypeptide may be biotinylated using an amine reactive biotinylation reagent such as, for example, EZ-Link Sulfo-NHS—SS-Biotin (sulfosuccinimidyl 2-(biotinamido)-ethyl-1,3-dithiopropionate; Pierce-Thermo Scientific, Rockford, Ill., USA; See, e.g., Jaiswal et al., Nature Biotech. 21:47-51 (2003)). For example, isolated erythroid cells may be incubated for 30 min at 4° C. in 1 mg/ml solution of sulfo-NHS-SS in phosphate-buffered saline. Excess biotin reagent is removed by washing the cells with Tris-buffered saline. The biotinylated cells are then reacted with the biotinylated exogenous antigenic polypeptide in the presence of streptavidin to form an aAPC presenting an exogenous antigen-presenting polypeptide that is bound to one or more exogenous antigenic polypeptides through a biotin-streptavidin bridge.

Erythroid cells described herein can also be produced using coupling reagents to link an exogenous polypeptide to a cell. For instance, click chemistry can be used. Coupling reagents can be used to couple an exogenous polypeptide to a cell, for example, when the exogenous polypeptide is a complex or difficult to express polypeptide, e.g., a polypeptide, e.g., a multimeric polypeptide; large polypeptide; polypeptide derivatized in vitro; an exogenous polypeptide that may have toxicity to, or which is not expressed efficiently in, the erythroid cells. Click chemistry and other conjugation methods for functionalizing erythroid cells is described in International Application No. PCT/US2018/000042, which claims priority to U.S. Provisional Application No. 62/460,589, filed Feb. 17, 2017 and U.S. Provisional Application No. 62/542,142, filed Jul. 8, 2017, the entire contents of which are incorporated herein by reference.

Thus, in some embodiments, an erythroid cell described herein comprises many as, at least, more than, or about 5,000, 10,000, 50,000, 100,000, 200,000, 300,000, 400,000, 500,000 coupling reagents per cell. In some embodiments, the erythroid cells are made by a method comprising a) coupling a first coupling reagent to an erythroid cell, thereby making a pharmaceutical preparation, product, or intermediate. In an embodiment, the method further comprises: b) contacting the cell with an exogenous polypeptide coupled to a second coupling reagent e.g., under conditions suitable for reaction of the first coupling reagent with the second coupling reagent. In some embodiments, two or more exogenous polypeptides are coupled to the cell (e.g., using click chemistry). In some embodiments, a first exogenous polypeptide is coupled to the cell (e.g., using click chemistry) and a second exogenous polypeptide comprises a polypeptide expressed from an exogenous nucleic acid.

In some embodiments, the coupling reagent comprises an azide coupling reagent. In some embodiments, the azide coupling reagent comprises an azidoalkyl moiety, azidoaryl moiety, or an azidoheteroaryl moiety. Exemplary azide coupling reagents include 3-azidopropionic acid sulfo-NHS ester, azidoacetic acid NHS ester, azido-PEG-NHS ester, azidopropylamine, azido-PEG-amine, azido-PEG-maleimide, bis-sulfone-PEG-azide, or a derivative thereof. Coupling reagents may also comprise an alkene moiety, e.g., a transcycloalkene moiety, an oxanorbornadiene moiety, or a tetrazine moiety. Additional coupling reagents can be found in Click Chemistry Tools (available on the world wide web at clickchemistrytools.com) or Lahann, J (ed) (2009) Click Chemistry for Biotechnology and Materials Science, each of which is incorporated herein by reference in its entirety.

In some embodiments, the exogenous antigenic polypeptide is attached to the cell, e.g., an erythroid cell, via a covalent attachment to generate an aAPC comprising an erythroid cell presenting one or more exogenous antigenic polypeptides (e.g. a first exogenous antigenic polypeptide, or a first antigenic polypeptide and a second exogenous antigenic polypeptide). For example, the antigenic polypeptide may be derivatized and bound to the erythroid cell or platelet using a coupling compound containing an electrophilic group that will react with nucleophiles on the erythroid cell or platelet to form the interbonded relationship. Representative of these electrophilic groups are αβ unsaturated carbonyls, alkyl halides and thiol reagents such as substituted maleimides. In addition, the coupling compound can be coupled to an antigenic polypeptide via one or more of the functional groups in the polypeptide such as amino, carboxyl and tryosine groups. For this purpose, coupling compounds should contain free carboxyl groups, free amino groups, aromatic amino groups, and other groups capable of reaction with enzyme functional groups. Highly charged antigenic polypeptides can also be prepared for immobilization on, e.g., erythroid cells or platelets through electrostatic bonding to generate a modified enucleated cell. Examples of these derivatives would include polylysyl and polyglutamyl enzymes.

The choice of the reactive group embodied in the derivative depends on the reactive conditions employed to couple the electrophile with the nucleophilic groups on the erythroid cell or platelet for immobilization. A controlling factor is the desire not to inactivate the coupling agent prior to coupling of the exogenous polypeptide immobilized by the attachment to the erythroid cell or platelet. Such coupling immobilization reactions can proceed in a number of ways. Typically, a coupling agent can be used to form a bridge between the exogenous polypeptide and the erythroid cell or platelet. In this case, the coupling agent should possess a functional group such as a carboxyl group which can be caused to react with the exogenous polypeptide. One way of preparing the exogenous polypeptide for conjugation includes the utilization of carboxyl groups in the coupling agent to form mixed anhydrides which react with the exogenous polypeptide, in which use is made of an activator which is capable of forming the mixed anhydride. Representative of such activators are isobutylchloroformate or other chloroformates which give a mixed anhydride with coupling agents such as 5,5′-(dithiobis(2-nitrobenzoic acid) (DTNB), p-chloromercuribenzoate (CMB), or m-maleimidobenzoic acid (MBA). The mixed anhydride of the coupling agent reacts with the exogenous polypeptide to yield the reactive derivative which in turn can react with nucleophilic groups on the erythroid cell or platelet to immobilize the exogenous polypeptide.

Functional groups on an exogenous antigenic polypeptide, such as carboxyl groups can be activated with carbodiimides and the like activators. Subsequently, functional groups on the bridging reagent, such as amino groups, will react with the activated group on the exogenous polypeptide to form the reactive derivative. In addition, the coupling agent should possess a second reactive group which will react with appropriate nucleophilic groups on the erythroid cell or platelet to form the bridge. Typical of such reactive groups are alkylating agents such as iodoacetic acid, αβ unsaturated carbonyl compounds, such as acrylic acid and the like, thiol reagents, such as mercurials, substituted maleimides and the like.

Alternatively, functional groups on the exogenous antigenic polypeptide can be activated so as to react directly with nucleophiles on, e.g., erythroid cells or platelets to obviate the need for a bridge-forming compound. For this purpose, use is made of an activator such as Woodward's Reagent K or the like reagent which brings about the formation of carboxyl groups in the exogenous polypeptide into enol esters, as distinguished from mixed anhydrides. The enol ester derivatives of exogenous polypeptides subsequently react with nucleophilic groups on, e.g., an erythroid cell or platelet to effect immobilization of the antigenic polypeptide, thereby creating a modified enucleated cell.

In some embodiments, the exogenous polypeptide (e.g. a first and/or a second exogenous polypeptide) is linked to a membrane anchor, such as GPA transmembrane domain (GPA), as a N-terminal or C-terminal fusion, e.g., such that the exogenous polypeptide is on the surface of the enucleated cell (e.g. modified enucleated cell).

In some embodiments, the aAPC comprising an erythroid cell or enucleated cell presenting one or more exogenous antigenic polypeptides is generated by contacting an erythroid cell or enucleated cell with an antigenic polypeptide and optionally a payload, wherein contacting does not include conjugating the antigenic polypeptide to the erythroid cell using an attachment site comprising Band 3 (CD233), aquaporin-1, Glut-1, Kidd antigen, RhAg/R1i50 (CD241), Rli (CD240), Rh30CE (CD240CE), Rh30D (CD240D), Kx, glycophorin B (CD235b), glycophorin C (CD235c), glycophorin D (CD235d), Kell (CD238), Duffy/DARCi (CD234), CR1 (CD35), DAF (CD55), Globoside, CD44, ICAM-4 (CD242), Lu/B-CAM (CD239), XG1/XG2 (CD99), EMMPRIN/neurothelin (CD147), JMH, Glycosyltransferase, Cartwright, Dombrock, C4A/CAB, Scianna, MER2, stomatin, BA-1 (CD24), GPIV (CD36), CD108, CD139, or H antigen (CD173).

In some embodiments, the aAPC comprises an engineered erythroid cell or enucleated cell presenting one or more exogenous antigenic polypeptides, wherein the one or more exogenous antigenic polypeptides are enzymatically conjugated onto the cell and/or to an exogenous antigen-presenting polypeptide.

In some embodiments, the engineered erythroid cell (e.g., engineered enucleated erythroid cell) or enucleated cell (e.g., modified enucleated cell) presents one or more exogenous antigenic polypeptides, wherein the one or more exogenous antigenic polypeptides are enzymatically conjugated to the exogenous antigen-presenting polypeptide (e.g., an exogenous antigen-presenting polypeptide comprising an HLA-E polypeptide or an HLA-G polypeptide).

In specific embodiments, the exogenous antigenic polypeptide can be conjugated to the exogenous antigen-presenting polypeptide by various chemical and enzymatic means provided herein, including but not limited to, chemical conjugation with bifunctional cross-linking agents such as, e.g., an NHS ester-maleimide heterobifunctional crosslinker to connect a primary amine group with a reduced thiol group. These methods also include enzymatic strategies such as, e.g., transpeptidase reaction mediated by a sortase enzyme to connect one polypeptide containing an acceptor sequence (e.g., LPXTG (SEQ ID NO: 6) or LPXTA (SEQ ID NO: 7)) with a polypeptide containing an N-terminal donor sequence (e.g., GG or GGG), see, e.g., Swee et al., Proc. Nat'l. Acad. Sci. USA 110 (4) 1428-1433 2013, the contents of which are hereby incorporated herein by reference. Additional acceptor sequences and donor sequences that may be used for sortase-mediated conjugation reactions, and methods utilizing sortagging are described in Antos et al. 2016, Curr Opin Struct Biol. 38; 111-118, the contents of which are hereby incorporated herein by reference. In some embodiments, the N-terminus of the exogenous antigen-presenting polypeptide comprises an N-terminal donor sequence GG or GGG. In some embodiments, N-terminal donor sequence GG or GGG on the exogenous antigen-presenting polypeptide is connected to an exogenous antigenic polypeptide containing the acceptor sequence LPXTG (SEQ ID NO: 6) or LPXTA (SEQ ID NO: 7), via a sortase-mediated reaction (e.g., a sortase A-mediated reaction).

In some embodiments, the exogenous antigenic polypeptide can be conjugated to the exogenous antigen-presenting polypeptide using Butelase 1, as described e.g., in Nguyen et al. 2016 Nature Protocols 11: 1977-88. Butelase 1 is a ligase from Clitoria ternatea (see, e.g., UniProtKB Accession No. A0A060D9Z7). Butelase 1-mediated cyclization requires a tripeptide recognition sequence Asx-His-Val (wherein Asx is Asp or Asn) at the C-terminus of one of the polypeptides being conjugated. Butelase 1 displays very loose specificity for the N-terminus of the second polypeptide being conjugated, wherein the N-terminus of the second polypeptide comprises X₁X₂, wherein X1 is any amino acid and X₂ is I, L, V, or C.

The methods provided herein to conjugate an exogenous antigenic polypeptide to a exogenous antigen-presenting polypeptide also include combination methods, such as e.g., sortase-mediated conjugation of Click Chemistry handles (an azide and an alkyne) on the antigen and the cell, respectively, followed by a cyclo-addition reaction to chemically bond the exogenous antigenic polypeptide to the antigen-presenting polypeptide, see e.g., Neves et al., Bioconjugate Chemistry, 24 (6), pp 934-941 2013. Sortase-mediated modification of proteins is described in International Application No. PCT/US2014/037545 and International Application No. PCT/US2014/037554, both of which are incorporated by reference in their entireties herein.

In some embodiments, a catalytic bond-forming polypeptide, such as a SpyTag/SpyCatcher system, can be used to attach the exogenous antigenic polypeptide to the exogenous antigen-presenting polypeptide. The SpyTag polypeptide sequence can be included on the extracellular surface of the engineered erythroid cells or enucleated cells. The SpyTag polypeptide can be, for example, fused to the N terminus of a exogenous antigen-presenting polypeptide. An antigenic polypeptide can be fused to SpyCatcher. Upon reaction of the SpyTag and SpyCatcher polypeptides, a covalent bond will be formed that attaches the exogenous antigenic polypeptide to the exogenous antigen-presenting polypeptide.

The engineered erythroid cells (e.g., engineered enucleated erythroid cells) or enucleated cells (e.g., modified enucleated cells), described herein can also be produced using one or more of the coupling reagents, as described herein, for e.g., click chemistry, to attach an exogenous antigenic polypeptide to an exogenous antigen-presenting polypeptide. In some embodiments, coupling reagents, for e.g., transglutaminase-mediated conjugation and fucosylation-mediated conjugation can be used to couple an exogenous antigenic polypeptide to an exogenous antigen-presenting polypeptide, wherein thereafter the complex of the antigen-presenting polypeptide bound to an exogenous antigenic polypeptide, can be attached to the surface of the cell, using one of the coupling reagents described herein, for e.g., click chemistry.

In specific embodiments, any of the exogenous polypeptides described herein (e.g., the exogenous antigenic polypeptide) can be conjugated to the surface of, e.g., an engineered erythroid cell or enucleated cell by various chemical and enzymatic means, including but not limited to chemical conjugation with bifunctional cross-linking agents such as, e.g., an NHS ester-maleimide heterobifunctional crosslinker to connect a primary amine group with a reduced thiol group. These methods also include enzymatic strategies such as, e.g., transpeptidase reaction mediated by a sortase enzyme to connect one polypeptide containing the acceptor sequence LPXTG (SEQ ID NO: 6) or LPXTA (SEQ ID NO: 7) with a polypeptide containing the N-terminal donor sequence GGG, see e.g., Swee et al., PNAS 2013. The methods also include combination methods, such as e.g., sortase-mediated conjugation of Click Chemistry handles (an azide and an alkyne) on the antigen and the cell, respectively, followed by a cyclo-addition reaction to chemically bond the antigen to the cell, see e.g., Neves et al., Bioconjugate Chemistry, 2013. Sortase-mediated modification of proteins is described in International Application No. PCT/US2014/037545 and International Application No. PCT/US2014/037554, both of which are incorporated by reference in their entireties herein.

In some embodiments, a protein is modified by the conjugation of a sortase substrate comprising an amino acid, a peptide, a protein, a polynucleotide, a carbohydrate, a tag, a metal atom, a contrast agent, a catalyst, a non-polypeptide polymer, a recognition element, a small molecule, a lipid, a linker, a label, an epitope, an antigen, a therapeutic agent, a toxin, a radioisotope, a particle, or moiety comprising a reactive chemical group, e.g., a click chemistry handle.

If desired, a catalytic bond-forming polypeptide domain can be expressed on or in e.g., an erythroid cell or platelet, either intracellularly or extracellularly. Many catalytic bond-forming polypeptides exist, including transpeptidases, sortases, and isopeptidases, including those derived from Spy0128, a protein isolated from Streptococcus pyogenes.

It has been demonstrated that splitting the autocatalytic isopeptide bond-forming subunit (CnaB2 domain) of Spy0128 results in two distinct polypeptides that retain catalytic activity with specificity for each other. The polypeptides in this system are termed SpyTag and SpyCatcher. Upon mixing, SpyTag and SpyCatcher undergo isopeptide bond formation between Asp117 on SpyTag and Lys31 on SpyCatcher (Zakeri and Howarth, JACS 2010, 132:4526). The reaction is compatible with the cellular environment and highly specific for protein/peptide conjugation (Zakeri, B. et al.; Fierer, J. O.; Celik, E.; Chittock, E. C.; Schwarz-Linek, U.; Moy, V. T.; Howarth, M. Proc. Natl. Acad. Sci. U.S.A. 2012, 109, E690-E697). SpyTag and SpyCatcher has been shown to direct post-translational topological modification in elastin-like protein. For example, placement of SpyTag at the N-terminus and SpyCatcher at the C-terminus directs formation of circular elastin-like proteins (Zhang et al, Journal of the American Chemical Society, 2013).

The components SpyTag and SpyCatcher can be interchanged such that a system in which molecule A is fused to SpyTag and molecule B is fused to SpyCatcher is functionally equivalent to a system in which molecule A is fused to SpyCatcher and molecule B is fused to SpyTag. For the purposes of this document, when SpyTag and SpyCatcher are used, it is to be understood that the complementary molecule could be substituted in its place.

A catalytic bond-forming polypeptide, such as a SpyTag/SpyCatcher system, can be used to attach an exogenous polypeptide (e.g., an antigenic polypeptide to the surface of, e.g., an erythroid cell, such as an engineered erythroid cell, to generate an aAPC. The SpyTag polypeptide sequence can be expressed on the extracellular surface of the erythroid cell. The SpyTag polypeptide can be, for example, fused to the N terminus of a type-1 or type-3 transmembrane protein, e.g., glycophorin A, fused to the C terminus of a type-2 transmembrane protein, e.g., Kell, inserted in-frame at the extracellular terminus or in an extracellular loop of a multi-pass transmembrane protein, e.g., Band 3, fused to a GPI-acceptor polypeptide, e.g., CD55 or CD59, fused to a lipid-chain-anchored polypeptide, or fused to a peripheral membrane protein. The nucleic acid sequence encoding the SpyTag fusion can be expressed within an aAPC. An antigenic polypeptide can be fused to SpyCatcher. The nucleic acid sequence encoding the SpyCatcher fusion can be expressed and secreted from the same erythroid cell that expresses the SpyTag fusion. Alternatively, the nucleic acid sequence encoding the SpyCatcher fusion can be produced exogenously, for example in a bacterial, fungal, insect, mammalian, or cell-free production system. Upon reaction of the SpyTag and SpyCatcher polypeptides, a covalent bond will be formed that attaches the antigenic polypeptide to the surface of the erythroid cell to form an aAPC. An engineered erythroid cell or enucleated cell comprising the antigenic polypeptide fusion is an example of an aAPC that comprises a conjugated antigenic polypeptide.

In some embodiments, the SpyTag polypeptide may be expressed as a fusion to the N terminus of glycophorin A under the control of the Gatal promoter in an engineered erythroid cell or enucleated cell. An exogenous polypeptide (e.g., exogenous antigenic polypeptides, exogenous antigen-presenting polypeptides, exogenous costimulatory polypeptides, exogenous coinhibitory polypeptides, cytokines, and exogenous Treg costimulatory polypeptides), fused to the SpyCatcher polypeptide sequence can be expressed under the control of the Gatal promoter in the same erythroid cell. Upon expression of both fusion polypeptides, an isopeptide bond will be formed between the SpyTag and SpyCatcher polypeptides, forming a covalent bond between the erythroid cell surface and the exogenous polypeptide.

In some embodiments, the SpyTag polypeptide may be expressed as a fusion to the N terminus of glycophorin A under the control of the Gatal promoter in an erythroid cell. An exogenous polypeptide (e.g., exogenous antigenic polypeptides, cytokines, exogenous costimulatory polypeptides, exogenous coinhibitory polypeptides, and exogenous Treg costimulatory polypeptides) fused to the SpyCatcher polypeptide sequence can be expressed in a suitable mammalian cell expression system, for example HEK293 cells. Upon expression of the SpyTag fusion polypeptide on the erythroid cell, the SpyCatcher fusion polypeptide can be brought in contact with the cell. Under suitable reaction conditions, an isopeptide bond will be formed between the SpyTag and SpyCatcher polypeptides, forming a covalent bond between the erythroid cell surface and the exogenous polypeptide. An engineered erythroid cell or enucleated cell comprising the antigenic polypeptide fusion is an example of an aAPC that comprises a conjugated antigenic polypeptide.

### IV. Methods of Using Artificial Antigen Presenting Cells

The present disclosure contemplates various methods of using the aAPCs described herein. As would be understood by one skilled in the art, based upon the disclosure provided herein, the dose and timing of administration of the aAPCs can be specifically tailored for each application described herein. More specifically, where it is desirable to provide immune modulation using certain molecules included on an aAPC, or several aAPCs. The skilled artisan, armed with the teachings provided herein and the knowledge available in the art, can readily determine the desired approach.

In one aspect, the disclosure features a method for activating an immune cell, e.g., a Treg cell, the method comprising contacting the cell with an aAPC engineered to activate an immune cell, e.g., a regulatory T cell (Treg), thereby activating the immune cell. In some embodiments, the aAPC comprises an exogenous antigen-presenting polypeptide comprising an HLA-E polypeptide and an exogenous antigenic polypeptide, e.g., a polypeptide selected from those listed in Table 1, e.g., an HSP60 peptide or a peptide derived from HSP60 (e.g., a leader sequence of HSP60).

In another aspect, the disclosure features a method of suppressing an immune cell, the method comprising contacting an immune cell with an aAPC engineered to suppress immune cell activity, thereby suppressing the immune cell. In some embodiments, the immune cell is a T cell, a B cell, an NK cell, a macrophage, or a dendritic cell. In some embodiments, the aAPC comprises an exogenous antigen-presenting polypeptide comprising an HLA-G polypeptide and an exogenous antigenic polypeptide. In some embodiments, the aAPC comprises an exogenous antigen-presenting polypeptide comprising an HLA-E polypeptide and an exogenous antigenic polypeptide.

The disclosure also encompasses a method for specifically inducing proliferation of an immune cell, e.g., a T cell, expressing a known costimulatory molecule. The method comprises contacting a population of immune cells comprising, e.g., at least one immune cell expressing the known costimulatory molecule, with an aAPC engineered to present a ligand of the costimulatory molecule. As disclosed elsewhere herein, where an aAPC expresses at least one costimulatory ligand that specifically binds with a costimulatory molecule on an immune cell, binding of the costimulatory molecule with its cognate costimulatory ligand induces proliferation of the immune cell. Thus, the immune cell of interest is induced to proliferate without having to first purify the cell from the population of cells. Also, this method provides a rapid assay for determining whether any cells in the population are expressing a particular costimulatory molecule of interest, since contacting the cells with the aAPC will induce proliferation and detection of the growing cells thereby identifying that an immune cell expressing a costimulatory molecule of interest was present in the sample. In this way, any immune cell of interest where at least one costimulatory molecule on the surface of the cell is known, can be expanded and isolated.

The disclosure also includes a method for specifically expanding an immune cell, e.g., a T cell, population subset. More particularly, the method comprises contacting a population of immune cells comprising at least one immune cell of a subset of interest with an aAPC capable of expanding that immune cell, or at least an aAPC expressing at least one costimulatory ligand that specifically binds with a cognate costimulatory molecule on the surface of the immune cell. Binding of the costimulatory molecule with its binding partner costimulatory ligand induces proliferation of the immune cell, thereby specifically expanding an immune cell population subset, e.g., a T cell population subset. One skilled in the art would understand, based upon the disclosure provided herein, that T cell subsets include T helper (T_{H1} and T_{H2}) CD4 expressing, cytotoxic T lymphocyte (CTL) (Tc1 or Tc2) T regulatory (TReg), T_{C/S}, naive, memory, central memory, effector memory, and γΔT cells. In some embodiments, the immune cell population is a T cell population, e.g., a T regulatory cell population. Therefore, cell populations enriched for a particular immune cell subset can be readily produced using the method of the disclosure.

In certain embodiments, immune cells, e.g., T cells, are expanded to between about 100 and about 1,000,000 fold, or between about 1,000 and about 1,000,000 fold, e.g., between 1,000 and about 100,000 fold.

The disclosure also includes a method for identifying a costimulatory ligand, or combination thereof, which specifically induces activation of an immune cell subset. Briefly, the method comprises contacting a population of immune cells with an aAPC presenting at least one costimulatory ligand, and comparing the level of proliferation of the immune cell subset contacted with the aAPC with the level of proliferation of an otherwise identical immune cell subset not contacted with the aAPC. A greater level of proliferation of the immune cell subset contacted with the aAPC compared with the level of proliferation of the otherwise identical immune cell subset which was not contacted with the aAPC is an indication that at the costimulatory ligand specifically induces activation of the immune cell subset to which that immune cell belongs.

The method permits the identification of a costimulatory ligand that specifically expands an immune cell subset where it is not previously known which factor(s) expand that immune cell subset. The skilled artisan would appreciate that in order to minimize the number of screenings, it is preferable to transduce as many nucleic acids encoding costimulatory ligands such that the number of assays can be reduced. Further, the method allows, by combining the various proteins (e.g., stimulatory ligand, costimulatory ligand, antigen, cytokine, and the like), to assess which combination(s) of factors will make the most effective aAPC, or combination of aAPCs, to expand the immune cell subset. In this way, the various requirements for growth and activation for each immune cell subset can be examined. Further, to evaluate immune cell expansion, CFSE staining can be used. In some embodiments, aAPCs are mixed with CD8+ T cells (e.g. from a subject suffering from a disease, such an autoimmune disease, an infectious disease, e.g., a viral infectious disease e.g., herpes stromal keratitis, Epstein Barr Virus, or HIV, or an allergic disease). To compare the initial rate of cell expansion, the cells are subject to CFSE staining to determine how well each aAPC induced the proliferation of all immune cells. CFSE staining provides a much more quantitative endpoint and allows simultaneous phenotyping of the expanded cells. Every day after stimulation, an aliquot of cells is removed from each culture and analyzed by flow cytometry. CFSE staining makes cells highly fluorescent. Upon cell division, the fluorescence is halved and thus the more times a cell divides the less fluorescent it becomes. The ability of each aAPC to induce immune cell, e.g., T cell proliferation is quantitated by measuring the number of cells that divided once, twice, three times and so on. The aAPC that induces the most number of cell divisions at a particular time point is deemed as the most potent expander.

To determine how well these aAPCs promote long-term growth of immune cells, cell growth curves are generated. These experiments are set up exactly as the CFSE experiments, but no CFSE is used. Every 2-3 days of culture, immune cells are removed from the respective cultures and counted using a Coulter counter which measures how many cells are present and the mean volume of the cells. The mean cell volume is the best predicator of when to restimulate the cells. In general, when immune cells are properly stimulated they triple their cell volume. When this volume is reduced to more than about half of the initial blast, it may be necessary to restimulate the immune cells to maintain a log linear expansion (Levine et al., 1996, Science 272:1939-1943; Levine et al., 1997, J. Immunol. 159:5921-5930). The time it takes each aAPC to induce 20 population doublings is calculated. The relative differences of each aAPC to induce this level of immune cell expansion is an important criteria on which a particular aAPC is used to move forward to clinical trials.

The phenotypes of the cells expanded by each aAPC are characterized to determine whether a particular subset is preferentially expanded. Prior to each restimulation, a phenotype analysis of the expanding immune cell populations is performed to define the differentiation state of the expanded immune cells using the CD27 and CD28 definitions proposed by Appay et al. (2002, Nature Med. 8, 379-385) and CCR7 definitions proposed by Sallusto et al. (1999, Nature 401:708-712). Perforin and Granzyme B intracellular staining are used to perform a gross measure to estimate cytolytic potential.

In one aspect, the method comprises contacting various aAPCs with the immune cell subset without first characterizing the costimulatory molecules on the surface of the immune cell subset. Also, the disclosure encompasses a method where the costimulatory molecule(s) present on the surface of the immune cell subset are examined prior to contacting the aAPCs with the cell. Thus, the present disclosure provides a novel assay for determining the growth requirements for various immune cell subsets, e.g., T cell subsets.

In another aspect, the disclosure features a method of treating a subject in need of an modulated or altered immune response, the method comprising contacting immune cells of the subject with an aAPC as described herein, thereby treating the subject in need of a modulated immune response. In some embodiments, the contacting is in vitro or in vivo. In some embodiments, the contacting is in vitro. In some embodiments, the contacting is in vivo.

In another aspect, the disclosure features a method of treating a subject in need of a modulated immune response, the method comprising a) determining an HLA status of the subject, b) selecting an artificial antigen presenting cell (aAPC) that is immunologically compatible with the subject, wherein the aAPC is an engineered erythroid cell expressing a first exogenous antigenic polypeptide, and c) administering the aAPC to the subject, thereby treating the subject in need of the modulated immune response.

In another aspect, the disclosure features a method of treating a subject in need of a modulated immune response, the method comprising a) determining an expression profile of an antigen in the subject, b) selecting an artificial antigen presenting cell (aAPC), wherein the aAPC is an engineered enucleated erythroid cell or enucleated cell including an exogenous antigen-presenting polypeptide and a first exogenous antigenic polypeptide, and c) administering the aAPC to the subject, thereby treating the subject in need of the modulated immune response.

In further aspects the present disclosure also provides methods for expanding a population of immune cells by cell surface moiety ligation. In addition, it is an object to provide a method of inducing a population of immune cells from a subject to rapidly proliferate exponentially for a long term to sufficient numbers for research purposes, comprising isolating a population of immune cells from a subject, activating the population of immune cells by contacting the immune cells ex vivo with at least one exogenous polypeptide that provides a primary activation signal to the immune cells; and stimulating the activated immune cells with at least one second exogenous polypeptide that provides a costimulatory signal, such that immune cells that have received a primary activation signal are stimulated to rapidly proliferate. In particular, it is an object to provide such a method when the subject is human, and wherein the method further comprises using the activated immune cells to identify antigens in the subject. Moreover, when the subject is infected with a disease or condition, having at least one antigen related thereto, the provided method further comprises using the activated immune cells to identify the at least one antigen. The antigen may comprise, e.g., and without limitation, a tumor antigen, an antigen relating to an autoimmune disease, or an infectious disease or pathogen. The method further comprises screening the at least one antigen as a target molecule for research purposes, or for developing a vaccine based upon the at least one antigen.

Methods of administering engineered enucleated erythroid cells and enucleated cells comprising (e.g., presenting) exogenous agent (e.g., polypeptides) are described, e.g., in WO2015/073587 and WO2015/153102, each of which is incorporated by reference in its entirety.

The methods described herein may include preparing a pharmaceutical composition comprising a plurality of the engineered enucleated erythroid cells or enucleated cells described herein.

Treatment of Conditions that would Benefit from Modulation of an Immune Cell Response

In some embodiments, the engineered erythroid cells (e.g., engineered enucleated erythroid cells) or enucleated cells (e.g., modified enucleated cells) are administered to a patient every 1, 2, 3, 4, 5, or 6 months.

In some embodiments, a dose of engineered erythroid cells (e.g., engineered enucleated erythroid cells) or enucleated cells (e.g., modified enucleated cells) comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, the engineered erythroid cells (e.g., engineered enucleated erythroid cells) or enucleated cells (e.g., modified enucleated cells) are administered to a patient in a dosing regimen (dose and periodicity of administration) sufficient to maintain function of the administered erythroid cells in the bloodstream of the patient over a period of 2 weeks to a year, e.g., one month to one year or longer, e.g., at least 2 weeks, 4 weeks, 6 weeks, 8 weeks, 3 months, 6 months, a year, 2 years.

In some embodiments, the engineered erythroid cells (e.g., engineered enucleated erythroid cells) or enucleated cells (e.g., modified enucleated cells) are administered to a patient in two doses or more (e.g., 2, 3, 4 or more doses). In further embodiments, the engineered erythroid cells (e.g., engineered enucleated erythroid cells) or enucleated cells (e.g., modified enucleated cells) are administered to a patient in two doses or more, wherein the second dose is administered at a time after the first dose when T-cell proliferation is determined to be at a peak. Peak T-cell proliferation can be determined using methods known to the skilled artisan. For example, peak T-cell proliferation can be determined by 3H-thymidine incorporation by proliferating T-cells, or by labelling proliferating T-cells with the fluorescent dye 5,6-carboxyfluorescein diacetate succinimidyl ester (CFSE).

In some aspects, the present disclosure provides a method of treating a disease or condition described herein, comprising administering to a subject in need thereof a composition described herein, e.g., an aAPC described herein. In some embodiments, the disease or condition is an autoimmune disease. In some embodiments, the disease or condition is an inflammatory disease. In some embodiments, the disease or condition is an infectious disease.

In some aspects, the disclosure provides a use of an aAPC described herein for treating a disease or condition described herein, e.g., an autoimmune disease, an inflammatory disease, an allergic disease, or an infectious disease. In some aspects, the disclosure provides a use of an aAPC described herein for manufacture of a medicament for treating a disease or condition described herein, e.g., an autoimmune disease, an inflammatory disease, an allergic disease, or an infectious disease.

Autoimmune Diseases

Over the past two decades, considerable progress has been made in the treatment of a range of autoimmune diseases with many patients enjoying an improvement in quality of life as a result. Despite their success, current therapeutic approaches to autoimmune diseases are either generally or specifically immunosuppressive, and expose patients to an increased risk of opportunistic infection and hematological cancers, as is the case with JAK inhibitors, anti-TNF antibodies and anti-CD20 targeted antibodies. In up to one-third of cases, patients with autoimmune diseases fail to respond to treatment, and most responding patients ultimately lose response over time.

While the triggers of most autoimmune diseases remain unknown, it is generally understood that clinical disease is the result of a loss of tolerance to one's own cells. The accepted model of disease assumes a genetic susceptibility triggered by an environmental event, which leads to a breakdown of T-cell-mediated immune suppression. In principle, restoration of peripheral tolerance should provide patients with a complete or partial cure.

A range of competitive approaches to peripheral tolerance restoration have been investigated over the years. These include the oral administration and direct injection of a protein or peptide with or without immunosuppression, the creation of peptide bearing nanoparticles and the adoptive transfer of engineered regulatory T cells. Thus far, these approaches have not proven to be successful in late stage clinical trials, but the field continues to progress. Direct administration of peptides and nanoparticles suffer biodistribution, stability, presentation and orientation challenges which limit the effectiveness of cell-to-cell signaling. To date, adoptive transfer approaches are all autologous and are hampered by some of the same handling and scalability issues that limit the application of other cellular therapies.

Thus, in certain embodiments, the aAPCs of the present disclosure provide a novel and improved method of treating autoimmune diseases. The methods provided for treating autoimmune diseases have numerous advantages, including, for example, effective presentation of an antigen-presenting polypeptide, e.g., an exogenous antigen-presenting polypeptide comprising an HLA-E polypeptide or an HLA-G polypeptide loaded with an exogenous antigenic polypeptide, and the extremely long half-life of aAPCs in the circulation, thus providing extended exposure to the properly presented antigen.

Accordingly, in certain embodiments, the present disclosure provides an aAPC comprising an erythroid cell (e.g. an enucleated erythroid cell) genetically engineered to include an exogenous antigen-presenting polypeptide comprising either a HLA-E polypeptide or a HLA-G polypeptide on the cell surface. In some embodiments, the antigen-presenting polypeptide comprises an HLA-E polypeptide bound to one or more exogenous antigenic polypeptides, wherein one of the one or more exogenous antigenic polypeptides comprises an antigenic polypeptide set forth in Table 1. In some embodiments, one of the one or more exogenous antigenic polypeptides comprises an HSP60 self antigen, or portion thereof, e.g., a leader sequence. In some embodiments, the antigen-presenting polypeptide comprises an HLA-G polypeptide bound to one or more exogenous antigenic polypeptides, wherein one of the one or more exogenous antigenic polypeptides comprises a peptide having the amino acid sequence motif XI/LPXXXXXL (SEQ ID NO: 8).

In another aspect, the aAPCs are designed to stimulate T regulatory cells, thereby biasing the immune system back to a more tolerogenic state. Thus, in some embodiments, the aAPC further comprises at least one costimulatory polypeptide as described herein. In this aspect, in preferred embodiments, the at least one exogenous costimulatory polypeptide expands regulatory T-cells (Tregs) and is, e.g., an exogenous Treg costimulatory polypeptide as described herein.

In yet another aspect, the aAPCs are designed to expand and stimulate T cells, e.g., cytotoxic CD8+ T cells. In this aspect, the autoimmune disease is preferably an autoimmune disease modulated or triggered by Tfh, Th1 and Th17 CD4 cells. In some embodiments, the aAPC further comprises at least one exogenous costimulatory polypeptide as described herein. In some embodiments, the at least one exogenous costimulatory polypeptide expands cytotoxic CD8+ T cells.

In some aspects, the disclosure provides a method of treating a subject having an autoimmune disease, comprising administering to the subject an effective number of the erythroid cells described herein to the subject, thereby treating the autoimmune disease. In such methods, the aAPC useful for treating the autoimmune disease comprises an exogenous antigen-presenting polypeptide and an antigenic polypeptide, wherein the antigenic polypeptide may be an antigenic polypeptide listed in Table 1, or antigenic-portion thereof, or a peptide having the amino acid sequence motif XI/LPXXXXXL (SEQ ID NO: 8).

Diseases of immune activation also include inflammatory diseases, such as, e.g. Crohn's disease, ulcerative colitis, celiac disease, or other idiopathic inflammatory bowel disease. Diseases of immune activation also include allergic diseases, such as, e.g. asthma, peanut allergy, shellfish allergy, pollen allergy, milk protein allergy, insect sting allergy, and latex allergy, animal dander allergy, black and English walnut allergy, brazil nut allergy, cashew nut allergy, chestnut allergy, dust mite allergy, egg allergy, fish allergy, hazelnut allergy, mold allergy, pollen allergy, grass allergy, shellfish allergy, soy allergy, tree nut allergy and wheat allergy, or an allergic disease mediated by T helper 2 (Th2) cells.

Diseases of immune activation also include immune activation in response to a therapeutic protein administered to treat a primary condition, that lessens the efficacy of the therapeutic protein, such as, e.g., clotting factor VIII in hemophilia A, clotting factor IX in hemophilia B, antitumor necrosis factor alpha (TNFα) antibodies in rheumatoid arthritis and other inflammatory diseases, glucocerebrosidase in Gaucher's disease, any recombinant protein used for enzyme replacement therapy, or asparaginase in acute lymphoblastic leukemia (ALL).

In some embodiments, a patient is suffering from an autoimmune disease or condition or a self-antibody mediated disease or condition, in which the patient's immune system is active against an endogenous (self) molecule, for example a protein antigen, such that the immune system attacks the endogenous molecule, induces inflammation, damages tissue, and otherwise causes the symptoms of the autoimmune or self-antibody disease or conditions. The immune response might be driven by antibodies that bind to the endogenous molecule, or it may be driven by overactive T cells that attack cells expressing the endogenous molecule, or it may be driven by other immune cells such as regulatory T cells, NK cells, NKT cells, or B cells. In these embodiments, an antigenic protein or a fragment thereof corresponding to the endogenous (self) molecule may be expressed on an aAPC comprising an erythroid cell, presenting one or more exogenous polypeptides, as described herein. The aAPCs, when administered once or more to the patient suffering from the disease or condition, would be sufficient to induce tolerance to the antigenic protein such that it no longer induced activation of the immune system, and thus would treat or ameliorate the symptoms of the underlying disease or condition. In certain embodiments, the aAPCs are used to stimulate T regulatory cells, thereby biasing the immune system back to a more tolerogenic state for the endogenous (self) molecule.

In other embodiments, a patient is suffering from an allergic disease, for example an allergy to animal dander, black walnut, brazil nut, cashew nut, chestnut, dust mites, egg, english walnut, fish, hazelnut, insect venom, latex, milk, mold, peanuts, pollen, grass, shellfish, soy, tree nuts, or wheat. A patient suffering from an allergy may mount an immune response upon contact with the antigenic fragment of the allergen, for example through diet, skin contact, injection, or environmental exposure. The immune response may involve IgE antibody, sensitized mast cells, degranulation, histamine release, and anaphylaxis, as well as canonical immune cells like T cells, B cells, dendritic cells, T regulatory cells, NK cells, neutrophils, and NKT cells. The allergic reaction may cause discomfort or it may be severe enough to cause death, and thus requires constant vigilance on the part of the sufferer as well as his or her family and caretakers. In these embodiments, the antigenic protein or a fragment thereof may be presented on an erythroid cell of the aAPC of the present disclosure. A population of these cells, when administered once or more to the patient suffering from the allergic disease or condition, would be sufficient to induce tolerance to the antigenic protein such that it no longer induced activation of the immune system upon exposure, and thus would treat or ameliorate the symptoms of the underlying allergic disease or condition.

In some embodiments, a patient is suffering from an infectious disease, e.g., a viral infectious disease.

### Subjects

The methods described herein are intended for use with any subject that may experience the benefits of these methods. Thus, “subjects,” “patients,” and “individuals” (used interchangeably) include humans as well as non-human subjects, particularly domesticated animals.

In some embodiments, the subject and/or animal is a mammal, eg., a human, mouse, rat, guinea pig, dog, cat, horse, cow, pig, rabbit, sheep, or non-human primate, such as a monkey, chimpanzee, or baboon. In other embodiments, the subject and/or animal is a non-mammal. In some embodiments, the subject and/or animal is a human. In some embodiments, the human is a pediatric human. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the disclosure pertains to veterinary use. In a specific embodiment, the non-human animal is a household pet. In another specific embodiment, the non-human animal is a livestock animal. In certain embodiments, the subject is a human cancer patient that cannot receive chemotherapy, e.g. the patient is unresponsive to chemotherapy or too ill to have a suitable therapeutic window for chemotherapy (e.g. experiencing too many dose- or regimen-limiting side effects). In certain embodiments, the subject is a human cancer patient having advanced and/or metastatic disease.

In some embodiments, the subject is selected for treatment with an aAPC comprising an engineered erythroid cell (e.g., engineered enucleated erythroid cell) or enucleated cell (e.g., modified enucleated cell) including one or more exogenous polypeptides of the present disclosure, e.g., an antigen-presenting polypeptide, e.g., HLA-E or HLA-G. In some embodiments, the subject is selected for treatment of an autoimmune disease, an inflammatory disease, an allergic disease, or an infectious disease, with an aAPC comprising an engineered erythroid cell (e.g., engineered enucleated erythroid cell) or enucleated cell (e.g., modified enucleated cell) including one or more exogenous polypeptides, e.g., an exogenous antigen-presenting polypeptide, e.g., an exogenous antigen-presenting polypeptide comprising a HLA-E polypeptide or a HLA-G polypeptide, of the present disclosure.

### V. Pharmaceutical Compositions

The present disclosure encompasses the preparation and use of pharmaceutical compositions comprising an aAPC of the disclosure as an active ingredient. Such a pharmaceutical composition may consist of the active ingredient alone, as a combination of at least one active ingredient (e.g., an effective dose of an aAPC) in a form suitable for administration to a subject, or the pharmaceutical composition may comprise the active ingredient and one or more pharmaceutically acceptable carriers, one or more additional (active and/or inactive) ingredients, or some combination of these.

In some embodiments, the disclosure features a pharmaceutical composition comprising a plurality of the aAPCs described herein, and a pharmaceutical carrier. In other embodiments, the disclosure features a pharmaceutical composition comprising a population of aAPCs as described herein, and a pharmaceutical carrier. It will be understood that any single aAPCs, plurality of aAPCs, or population of aAPCs as described elsewhere herein may be present in a pharmaceutical composition as described herein.

In some embodiments, the pharmaceutical compositions provided herein comprise engineered or modified erythroid cells and non-engineered or unmodified erythroid cells. For example, a single unit dose of aAPCs can comprise, in various embodiments, about, at least, or no more than 10%, 20%, 30%, 40%, 50%, 60%, 70%, 75%, 80%), 85%, 90%, 95%, or 99% engineered erythroid cells or modified erythroid ells, wherein the remaining erythroid cells in the composition are not engineered or not modified.

In some embodiments, the pharmaceutical compositions provided herein comprise aAPCs comprising engineered enuclated erythroid cells or enucleated cells (e.g., modified enucleated cells) together with nucleated erythroid cells. For example, a single unit dose of engineered erythroid cells (e.g., enucleated and nucleated erythroid cells) can comprise, in various embodiments, about, or at least 10%, 20%, 30%, 40%, 50%, 60%, 70%, 75%, 80%, 85%, 90%, 91%, 92%, 93%, 94%, 95%, 96%, 97%, 98%, or 99% enucleated erythroid cells, wherein the remaining erythroid cells in the composition are nucleated.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The administration of the pharmaceutical compositions may be carried out in any convenient manner, including by aerosol inhalation, injection, ingestion, transfusion, implantation or transplantation. The compositions of the present disclosure may be administered to a patient subcutaneously, intradermally, intramuscularly, by intravenous (i.v.) injection, or intraperitoneally. The pharmaceutical compositions may be injected directly into a tumor or lymph node.

As used herein, the term “pharmaceutically acceptable carrier” means a chemical composition with which the active ingredient may be combined and which, following the combination, can be used to administer the active ingredient to a subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the disclosure may be prepared, packaged, or sold in formulations suitable for oral, rectal, vaginal, parenteral, topical, pulmonary, intranasal, intra-lesional, buccal, ophthalmic, intravenous, intra-organ or another route of administration. Other contemplated formulations include projected nanoparticles, liposomal preparations, resealed erythrocytes containing the active ingredient, and immunologically-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents. Particularly contemplated additional agents include anti-emetics and scavengers such as cyanide and cyanate scavengers and AZT, protease inhibitors, reverse transcriptase inhibitors, interleukin-2, interferons, cytokines, and the like.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The aAPC of the disclosure and/or T cells expanded using the aAPC, can be administered to an animal, preferably a human. When the T cells expanded using an aAPC of the disclosure are administered, the amount of cells administered can range from about 1 million cells to about 300 billion. Where the aAPCs themselves are administered, either with or without T cells expanded thereby, they can be administered in an amount ranging from about 100,000 to about one billion cells wherein the cells are infused into the animal, preferably, a human patient in need thereof. While the precise dosage administered will vary depending upon any number of factors, including but not limited to, the type of animal and type of disease state being treated, the age of the animal and the route of administration.

The aAPC may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

An aAPC (or cells expanded thereby) may be co-administered with the various other compounds (cytokines, chemotherapeutic and/or antiviral drugs, among many others). Alternatively, the compound(s) may be administered an hour, a day, a week, a month, or even more, in advance of the aAPC (or cells expanded thereby), or any permutation thereof. Further, the compound(s) may be administered an hour, a day, a week, or even more, after administration of aAPC (or cells expanded thereby), or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds and the aAPC (or cells expanded thereby), and the like.

Further, it would be appreciated by one skilled in the art, based upon the disclosure provided herein, that where the aAPC is to be administered to a mammal, the cells are treated so that they are in a “state of no growth”; that is, the cells are incapable of dividing when administered to a mammal. As disclosed elsewhere herein, the cells can be irradiated to render them incapable of growth or division once administered into a mammal. Other methods, including haptenization (e.g., using dinitrophenyl and other compounds), are known in the art for rendering cells to be administered, especially to a human, incapable of growth, and these methods are not discussed further herein. Moreover, the safety of administration of aAPC that have been rendered incapable of dividing in vivo has been established in Phase I clinical trials using aAPC transfected with plasmid vectors encoding some of the molecules discussed herein.

### Combination Therapies

In some embodiments, the disclosure provides methods that further comprise administering an additional agent to a subject. In some embodiments, the disclosure pertains to co-administration and/or co-formulation.

In some embodiments, administration of the aAPC acts synergistically when co-administered with another agent and is administered at doses that are lower than the doses commonly employed when such agents are used as monotherapy.

In some embodiments of the above aspects and embodiments, the erythroid cell is an enucleated erythroid cell. In some embodiments of the above aspects and embodiments, the erythroid cell is a nucleated erythroid cell.

In some embodiments, the disclosure features a pharmaceutical composition comprising a plurality of the engineered erythroid cells described herein, and a pharmaceutical carrier. In other embodiments, the disclosure features a pharmaceutical composition comprising a population of engineered erythroid cells as described herein, and a pharmaceutical carrier. It will be understood that any single engineered erythroid cell, plurality of engineered erythroid cells, or population of engineered erythroid cells as described elsewhere herein may be present in a pharmaceutical composition of the as described herein.

In some embodiments, the pharmaceutical compositions provided herein comprise engineered (i.e., modified) erythroid cells and unmodified erythroid cells. For example, a single unit dose of erythroid cells (e.g., modified and unmodified erythroid cells) can comprise, in various embodiments, about, at least, or no more than 10%, 20%, 30%, 40%, 50%, 60%, 70%, 75%, 80%), 85%, 90%, 95%, or 99% engineered erythroid cells, wherein the remaining erythroid cells in the composition are not engineered.

In some embodiments, the pharmaceutical compositions provided herein comprise engineered enucleated erythroid cells and nucleated erythroid cells. For example, a single unit dose of engineered erythroid cells (e.g., enucleated and nucleated erythroid cells) can comprise, in various embodiments, about, or at least 10%, 20%, 30%, 40%, 50%, 60%, 70%, 75%, 80%, 85%, 90%, 91%, 92%, 93%, 94%, 95%, 96%, 97%, 98%, or 99% enucleated erythroid cells, wherein the remaining erythroid cells in the composition are nucleated.

### VI. Kits

The disclosure includes various kits which comprise an aAPC of the disclosure, an applicator, and instructional materials which describe use of the kit to perform the methods of the disclosure. Although exemplary kits are described below, the contents of other useful kits will be apparent to the skilled artisan in light of the present disclosure. Each of these kits is included within the disclosure.

The disclosure includes a kit for specifically inducing activation of T regulatory cells using aAPCs including an exogenous antigen-presenting polypeptide comprising an HLA-E polypeptide, or inhibition of other immune cells, e.g., T cells, B cells, NK cells, macrophages, and/or dendritic cells using aAPCs including an exogenous antigen-presenting polypeptide comprising a HLA-E polypeptide or a HLA-G polypeptide. The kit is used pursuant to the methods disclosed in the disclosure. Briefly, the kit may be used to administer an aAPC of the disclosure to an immune cell, e.g., T regulatory cells, T cells, B cells, NK cells, macrophages, and/or dendritic cells. Further, the immune cells produced using this kit can be administered to an animal to achieve therapeutic results.

The kit further comprises an applicator useful for administering the aAPC to the immune cells. The particular applicator included in the kit will depend on, e.g., the method used to administer the aAPC, as well as the cells expanded by the aAPC, and such applicators are well-known in the art and may include, among other things, a pipette, a syringe, a dropper, and the like. Moreover, the kit comprises an instructional material for the use of the kit. These instructions simply embody the disclosure provided herein.

The kit includes a pharmaceutically-acceptable carrier. The composition is provided in an appropriate amount as set forth elsewhere herein. Further, the route of administration and the frequency of administration are as previously set forth elsewhere herein.

The kit encompasses an aAPC comprising a wide plethora of molecules, such as, but not limited to, those set forth herein. However, the skilled artisan armed with the teachings provided herein, would readily appreciate that the disclosure is in no way limited to these, or any other, combination of molecules. Rather, the combinations set forth herein are for illustrative purposes and they in no way limit the combinations encompassed by the present disclosure.

All publications and patent applications cited in this specification are herein incorporated by reference in their entirety for all purposes as if each individual publication or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior disclosure or for any other reason.

### EXAMPLES

### Example 1. Generation of Erythroid Cells Genetically Engineered to Include an HLA-E-GPA Fusion Protein

Erythroid cells are transduced to include a fusion protein comprising an exogenous antigen-presenting polypeptide, specifically an exogenous antigen-presenting polypeptide comprising an HLA-E polypeptide fused to full length glycophorin A (GPA) protein, which serves as a membrane anchor (HLA-E-GPA fusion protein). For example, the exogenous antigen-presenting polypeptide comprising an HLA-E polypeptide fused to GPA could be arranged as depicted in FIG. 1, which provides an exemplary design for including one or more alpha domains (e.g., alpha1, alpha2, and alpha3 domains) of an HLA-E polypeptide linked to β2M polypeptide and to the GPA membrane anchor.

Cell culture and transduction is performed as described in the “Methods” section below to yield erythroid cells including HLA-E on the cell surface, anchored with GPA.

Binding of an APC-labelled or PE-labelled anti-HLA-E antibody is used to validate inclusion of the exogenous antigen-presenting polypeptide comprising an HLA-E polypeptide in the engineered erythroid cells.

### Methods

Production of Lentiviral Vector

The gene encoding the HLA-E-GPA fusion protein is cloned into the multiple cloning site of lentivirus vector pCDH with the MSCV promoter sequence from System Biosciences. Lentivirus is produced in 293T cells by transfecting the cells with pPACKH1 (System Biosciences) and pCDH lentivirus vector containing the nucleic acid encoding the HLA-E-GPA fusion protein. Cells are then placed in fresh culturing medium. The virus supernatant is collected 48 hours post-medium change by centrifugation at 1,500 rpm for 5 minutes. The supernatant is collected and frozen in aliquots at −80° C.

Expansion and Differentiation of Erythroid Cells

Human CD34+ cells derived from mobilized peripheral blood cells from normal human donors are purchased frozen from AllCells Inc. The expansion/differentiation procedure comprises 3 stages. In the first stage, thawed CD34+ erythroid precursors are cultured in Iscove's MDM medium comprising recombinant human insulin, human transferrin, recombinant human recombinant human stem cell factor, and recombinant human interleukin 3. In the second stage, erythroid cells are cultured in Iscove's MDM medium supplemented with serum albumin, recombinant human insulin, human transferrin, human recombinant stem cell factor, human recombinant erythropoietin, and L-glutamine. In the third stage, erythroid cells are cultured in Iscove's MDM medium supplemented with human transferrin, recombinant human insulin, human recombinant erythropoietin, and heparin. The cultures are maintained at 37° C. in 5% CO2 incubator.

Transduction of Erythroid Precursor Cells Erythroid precursor cells are transduced during step 1 of the culture process described above. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

Antibody Binding Binding of an APC-labelled or PE-labelled anti-HLA-E antibody is used to validate inclusion of the exogenous antigen-presenting polypeptide comprising an HLA-E polypeptide in the engineered erythroid cells. Binding of the antibody is measured by flow cytometry for APC fluorescence or PE fluorescence. A gate is set based on stained untransduced cells.

### Example 2. Generation of Erythroid Cells Genetically Engineered to Include an HLA-G-GPA Fusion Protein

### Results

Erythroid cells are transduced to include a fusion protein comprising an exogenous antigen-presenting polypeptide, specifically an exogenous antigen-presenting polypeptide comprising an HLA-G polypeptide fused to GPA protein, which serves as a membrane anchor (HLA-G-GPA fusion protein). For example, the exogenous antigen-presenting polypeptide comprising an HLA-G polypeptide fused to GPA could be arranged as depicted in FIG. 2B, which provides an exemplary design for including the one or more alpha domains (e.g., alpha1, alpha2, and alpha3 domains) of an HLA-G polypeptide linked to the GPA membrane anchor and to a β2M polypeptide.

Cell culture and transduction is performed as described in the “Methods” section below to yield erythroid cells including an exogenous antigen-presenting polypeptide comprising an HLA-G polypeptide on the cell surface, anchored with a GPA transmembrane domain.

Binding of an APC-labelled or PE-labelled anti-HLA-G antibody is used to validate inclusion of the exogenous antigen-presenting polypeptide comprising an HLA-G polypeptide in the engineered erythroid cells.

### Methods

Production of Lentiviral Vector

The gene encoding the HLA-G-GPA fusion protein is cloned into the multiple cloning site of lentivirus vector pCDH with the MSCV promoter sequence from System Biosciences. Lentivirus is produced in 293T cells by transfecting the cells with pPACKH1 (System Biosciences) and pCDH lentivirus vector containing the nucleic acid encoding the HLA-G-GPA fusion protein. Cells are then placed in fresh culturing medium. The virus supernatant is collected 48 hours post-medium change by centrifugation at 1,500 rpm for 5 minutes. The supernatant is collected and frozen in aliquots at −80° C.

Expansion and Differentiation of Erythroid Cells

Human CD34+ cells derived from mobilized peripheral blood cells from normal human donors are purchased frozen from AllCells Inc. The expansion/differentiation procedure comprises 3 stages. In the first stage, thawed CD34+erythroid precursors are cultured in Iscove's MDM medium comprising recombinant human insulin, human transferrin, recombinant human recombinant human stem cell factor, and recombinant human interleukin 3. In the second stage, erythroid cells are cultured in Iscove's MDM medium supplemented with serum albumin, recombinant human insulin, human transferrin, human recombinant stem cell factor, human recombinant erythropoietin, and L-glutamine. In the third stage, erythroid cells are cultured in Iscove's MDM medium supplemented with human transferrin, recombinant human insulin, human recombinant erythropoietin, and heparin. The cultures are maintained at 37° C. in 5% CO2 incubator.

Transduction of Erythroid Precursor Cells

Erythroid precursor cells are transduced during step 1 of the culture process described above. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

Antibody Binding

Binding of an APC-labelled or PE-labelled anti-HLA-G antibody is used to validate inclusion of the exogenous antigen-presenting polypeptide comprising a HLA-G polypeptide in the engineered erythroid cells. Binding of the antibody is measured by flow cytometry for APC fluorescence or PE fluorescence. A gate is set based on stained untransduced cells.

### Example 3. Generation of Erythroid Cells Genetically Engineered to Include an HSP60-HLA-E-GPA Fusion Protein

### Results

Erythroid cells are transduced to include a fusion protein comprising an exogenous antigenic polypeptide, the leader sequence of HSP60, fused to an exogenous antigen-presenting polypeptide, specifically an exogenous antigen-presenting polypeptide comprising an HLA-E polypeptide fused to GPA, which serves as a membrane anchor (HSP60-HLA-E-GPA fusion protein). FIG. 1 shows a schematic of an exemplary design for a antigenic polypeptide, e.g., the HSP60 leader sequence peptide, and an exogenous antigen-presenting polypeptide comprising an HLA-E polypeptide as a single chain fusion, where the exogenous peptide (HSP60 leader sequence) is linked to a β2M polypeptide, which is linked to one or more alpha domains (e.g., alpha1, alpha2, and alpha3 domains) of an HLA-E polypeptide, which is linked to the GPA membrane anchor. Cell culture and transduction is performed as described in the “Methods” section below to yield erythroid cells including the leader sequence of HSP60 presented by an exogenous antigen-presenting polypeptide comprising an HLA-E polypeptide on the cell surface, anchored with a GPA transmembrane domain.

Binding of an APC-labelled or PE-labelled anti-HLA-E antibody is used to validate inclusion of the exogenous antigen-presenting polypeptide comprising a HLA-E polypeptide in the engineered erythroid cells.

### Methods

Production of Lentiviral Vector

The gene encoding the HSP60-HLA-E-GPA fusion protein is cloned into the multiple cloning site of lentivirus vector pCDH with the MSCV promoter sequence from System Biosciences. Lentivirus is produced in 293T cells by transfecting the cells with pPACKH1 (System Biosciences) and pCDH lentivirus vector containing the HSP60-HLA-E-GPA gene. Cells are then placed in fresh culturing medium. The virus supernatant is collected 48 hours post-medium change by centrifugation at 1,500 rpm for 5 minutes. The supernatant is collected and frozen in aliquots at −80° C.

Expansion and Differentiation of Erythroid Cells

Human CD34+ cells derived from mobilized peripheral blood cells from normal human donors are purchased frozen from AllCells Inc. The expansion/differentiation procedure comprises 3 stages. In the first stage, thawed CD34+erythroid precursors are cultured in Iscove's MDM medium comprising recombinant human insulin, human transferrin, recombinant human recombinant human stem cell factor, and recombinant human interleukin 3. In the second stage, erythroid cells are cultured in Iscove's MDM medium supplemented with serum albumin, recombinant human insulin, human transferrin, human recombinant stem cell factor, human recombinant erythropoietin, and L-glutamine. In the third stage, erythroid cells are cultured in Iscove's MDM medium supplemented with human transferrin, recombinant human insulin, human recombinant erythropoietin, and heparin. The cultures are maintained at 37° C. in 5% CO2 incubator.

Transduction of Erythroid Precursor Cells

Erythroid precursor cells are transduced during step 1 of the culture process described above. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

Antibody Binding

Binding of an APC-labelled or PE-labelled anti-HLA-E antibody is used to validate inclusion of the exogenous antigen-presenting polypeptide comprising an HLA-E polypeptide in the engineered erythroid cells. Binding of the antibody is measured by flow cytometry for APC fluorescence or PE fluorescence. A gate is set based on stained untransduced cells.

### Example 4. Activation of CD8+ T Regulatory Cells In Vitro by Erythroid Cells Including an HLA-E-HSP60 Fusion Protein

Erythroid cells are transduced to include a fusion protein comprising an exogenous antigenic polypeptide, the leader sequence of HSP60, fused to an exogenous antigen-presenting polypeptide comprising an HLA-E polypeptide fused to the GPA transmembrane domain (HSP60-HLA-E-GPA fusion protein), as described in Example 3.

Functional activity is assessed using a human CD8+ T-regulatory-cell-based proliferative assay. Cells are labeled with the fluorescent dye 5,6-carboxyfluorescein diacetate succinimidyl ester (CFSE). Those cells that proliferate in response to the engineered erythrocytes show a reduction in CFSE fluorescence intensity, which is measured directly by flow cytometry. Alternatively radioactive thymidine incorporation can be used to assess the rate of growth of the CD8+ T regulatory cells stimulated with erythroid cells engineered to include a HSP60-HLA-E-GPA fusion protein.

Functional activity is also assessed using an in vitro Treg suppression assay. Such an assay is described in Collinson and Vignali (Methods Mol Biol. 2011; 707: 21-37, incorporated by reference in its entirety herein).

### Example 5. Testing Erythroid Cells Genetically Engineered to Include an HLA-E-HSP60-GPA Fusion Protein in an In Vitro Model of Type 1 Diabetes

Erythroid cells are transduced to include a fusion protein comprising an exogenous antigenic polypeptide, the leader sequence of HSP60, fused to an exogenous antigen-presenting polypeptide comprising an HLA-E polypeptide fused to the GPA transmembrane domain (HSP60-HLA-E-GPA fusion protein), as described in Example 3.

Functional activity is assessed using an in vitro self/non-self-discrimination assay, using the cells from Type 1 diabetes (T1D) patients with a defect in their CD8+ T regulatory cells, and compared to a normal control group with regard to their ability to specifically inhibit HLA-E-including targets loaded with HSP60. The assay is described in Jiang et al. (J. Clin Invest., 2010, 120(10):3641-3650, incorporated by reference in its entirety herein). Briefly, CD8+ T regulatory cells from T1D patients are boosted in vitro by priming with erythroid cells including an HLA-E-HSP60-GPA fusion protein, or a similar construct lacking the exogenous antigenic polypeptide (control). The boosted CD8+ T regulatory cell lines are tested for their specificity in a CD8+ T cell inhibition assay.

### Example 6. Testing Erythroid Cells Engineered to Include an HLA-E-HSP60-GPA Fusion Protein in a Mouse CIA Model

Rheumatoid arthritis (RA) is an autoimmune disease characterized primarily by joint inflammation and erosion. More effective approaches to RA may require elimination of autoreactive T cells, which, in turn depends on a precise definition of the T regulatory cells that may target and eliminate the pathogenic T cell subsets that induce disease. The contribution of T regulatory and effector cells to autoimmune arthritis, can be studied in the well-established collagen-induced arthritis (CIA) mouse model. This murine disease model shares several similarities with human RA, including breach of self-tolerance, generation of autoantibodies, inflammatory changes in multiple joints, and erosion of bone and cartilage accompanied by pannus formation.

Murine erythroid cells are conjugated with an exogenous antigen-presenting polypeptide HLA-E presenting the leader sequence of HSP60 peptide (HLA-E-HSP60-GPA fusion protein), using the click methodology (click chemistry for functionalizing erythroid cells is described in International Application No. PCT/US2018/000042, which claims priority to U.S. Provisional Application No. 62/460,589, filed Feb. 17, 2017 and U.S. Provisional Application No. 62/542,142, filed Jul. 8, 2017, incorporated by reference in their entireties herein).

Functional activity is assessed in vivo using a mouse CIA model, as described in Leavenworth et al. (J Clin Invest. 2013; 123(3):1382-1389, incorporated by reference in its entirety herein). Briefly, CIA would be induced in B6 mice ages 8-12 weeks by intraderminally injecting 150 μg of cCII emulsified in CFA followed by a boost 21 days later with 100 μg of cCII emulsified in IFA. Dosing with erythroid cells conjugated with the exogenous antigen-presenting polypeptide comprising an HLA-E polypeptide presenting the leader sequence of the HPS60 peptide would initiate on day 27 (7 days after boost) through tail vein intravenous administration. Doses are administered 1 or more times over the span of the study which can range from 2-14 days between dosing. CIA arthritis scoring and autoantibody production are used as measurements of disease progression. Ex vivo analysis of CD8+ T regulatory cell numbers and phenotype also performed. CD8+ T regulatory cells profiled by CD8+CD122+Ly49+ and HSP60 tetramer positivity.

An alternative method of evaluation would be through adoptive transfer of in vitro expanded CD8+ T regulatory cells. Briefly, erythroid cells including an HLA-E-HSP60-GPA fusion protein are used to expand the HSP60-specific CD8+ T regulatory cells in vitro. Adoptive transfer of a small number (5×10⁴/mouse) of HSP60-HLA-E-GPA specific CD8+ T regulatory cells, into CIA mice are used to monitor the autoantibody production and progression of autoimmune arthritis.

CIA scoring is performed 2-3 times per week with the following scoring system: 0, normal; 1, mild swelling and/or erythema of the mid-foot or ankle joint, 2, moderate edematous swelling extending from ankle to the metatarsal joints; and 3, pronounced swelling encompassing the ankle, foot, and digits. Each limb is graded with a maximum score of 12 per mouse.

### Example 7. Testing Erythroid Cells Engineered to Include an HLA-E-HSP60-GPA Fusion Protein in a Mouse EAE Model

Experimental autoimmune encephalomyelitis (EAE) is the model most commonly used to study efficacy of potential drugs for treatment of multiple sclerosis (MS). Because of its many similarities to MS, EAE is used to study pathogenesis of autoimmunity, CNS inflammation, demyelination, cell trafficking and tolerance induction. EAE is characterized by paralysis (in some models the paralysis is relapsing-remitting), CNS inflammation and demyelination. Hooke Kits™ for EAE Induction in C57BL/6 Mice (Hooke Laboratories) are used to induce EAE in female C57BL/6 mice. Using this method, EAE is induced in C57BL/6 mice by immunization with an emulsion of MOG35-55 or MOG1-125 in complete Freund's adjuvant (CFA), followed by administration of pertussis toxin in PBS, first on the day of immunization and then again the following day. Typical EAE onset is 9 to 14 days after immunization, with peak of disease 3 to 5 days after onset for each mouse. The peak lasts 1 to 3 days, followed by partial recovery. Around 25% of mice will show an increase in EAE severity (relapse) after initial partial recovery. This usually occurs 20-27 days after immunization. Groups of 10 to 12 mice, with 4 to 6 mice per cage are used.

Erythroid cells comprising HSP60-HLA-E-GPA fusion protein are made as described in Example 6, and are formulated in a buffer appropriate for erythroid cells. Treatment with the enucleated erythroid cells begins at the time of EAE onset. If the erythroid cells are being tested for their ability to reverse the course of chronic EAE, treatment is initiated 7-14 days after disease onset. Mice are assigned to treatment groups as they develop EAE (rolling enrollment) or at a fixed time after immunization, but always in a balanced manner to achieve groups with similar time of EAE onset and similar EAE onset scores. If enrollment is after EAE onset, mice are also balanced for maximum score before enrollment.

Dosing occurs at a clinical score=1, as set out in Table 8, below.

Dosing of the animals may be carried out a total of 1 to 3 times (e.g., 2-14 days between doses.

Assessment of the effects of engineered enucleated erythroid cells including HSP60-HLA-E-GPA are determined using one or more of (1) EAE scoring; (2) change in body weight and (3) histological analysis, as described in detail below.

EAE Scoring

Typically, EAE is scored on scale of 0 to 5, including “in-between” scores (i.e. 0.5, 1.5, 2.5, 3.5) when the clinical picture lies between two defined scores. The scoring method differs slightly depending on the stage of disease (onset/peak vs. recovery), for each individual mouse. To avoid unconscious bias in scoring, mice are scored blind, by a person unaware of which mice have received which treatment.

TABLE 8
Mouse EAE scoring- onset and peak

| Score | Clinical observations |
|---|---|
| 0.0 | No obvious changes in motor function compared to non- |
| | immunized mice. When picked up by base of tail, the tail has |
| | tension and is erect. Hind legs are usually spread apart. When |
| | the mouse is walking, there is no gait or head tilting. |
| 0.5 | Tip of tail is limp. |
| | When picked up by base of tail, the tail has tension except for |
| | the tip. Muscle straining is felt in the tail, while the tail |
| | continues to move. |
| 1.0 | Limp tail. |
| | When picked up by base of tail, instead of being erect, the whole |
| | tail drapes over finger. Hind legs are usually spread apart. |
| | No signs of tail movement are observed. |
| 1.5 | Limp tail and hind leg inhibition. |
| | When picked up by base of tail, the whole tail drapes over finger. |
| | When the mouse is dropped on a wire rack, at least one hind leg |
| | falls through consistently. Walking is very slightly wobbly. |
| 2.0 | Limp tail and weakness of hind legs. |
| | When picked up by base of tail, the legs are not spread apart, but |
| | held closer together. When the mouse is observed walking, it has a |
| | clearly apparent wobbly walk. One foot may have toes dragging, |
| | but the other leg has no apparent inhibitions of movement. |
| | - OR - |
| | Mouse appears to be at score 0.0, but there are obvious signs of |
| | head tilting when the walk is observed. The balance is poor. |
| 2.5 | Limp tail and dragging of hind legs. |
| | Both hind legs have some movement, but both are dragging at the |
| | feet (mouse trips on hind feet). |
| | - OR - |
| | No movement in one leg/completely dragging one leg, but |
| | movement in the other leg. |
| | - OR - |
| | EAE severity appears mild when picked up (as score 0.0-1.5), but |
| | there is a strong head tilt that causes the mouse to occasionally fall |
| | over. |
| 3.0 | Limp tail and complete paralysis of hind legs (most common). |
| | - OR - |
| | Limp tail and almost complete paralysis of hind legs. One or both |
| | hind legs are able to paddle, but neither hind leg is able to move |
| | forward of the hind hip. |
| | - OR - |
| | Limp tail with paralysis of one front and one hind leg. |
| | - OR - |
| | ALL of: |
| | Severe head tilting, |
| | Walking only along the edges of the cage, |
| | Pushing against the cage wall, |
| | Spinning when picked up by base of tail. |
| 3.5 | Limp tail and complete paralysis of hind legs. In addition to: |
| | Mouse is moving around the cage, but when placed on its side, is |
| | unable to right itself. Hind legs are together on one side of body. |
| | - OR - |
| | Mouse is moving around the cage, but the hind quarters are flat |
| | like a pancake, giving the appearance of a hump in the front |
| | quarters of the mouse. |
| 4.0 | Limp tail, complete hind leg and partial front leg paralysis. |
| | Mouse is minimally moving around the cage but appears alert and |
| | feeding. Often euthanasia is recommended after the mouse scores |
| | 4.0 for 2 days. However, with daily s.c. fluids most C57BL/6 mice |
| | may recover to 3.5 or 3.0, while SJL mice may fully recover even |
| | if they reach score 4.0 at the peak of disease. When the mouse is |
| | euthanized because of severe paralysis, a score of 5.0 is entered |
| | for that mouse for the rest of the experiment. |
| 4.5 | Complete hind and partial front leg paralysis, no movement |
| | around the cage. Mouse is not alert. |
| | Mouse has minimal movement in the front legs. The mouse |
| | barely responds to contact. |
| | Euthanasia is recommended. When the mouse is euthanized |
| | because of severe paralysis, a score of 5.0 is entered for that |
| | mouse for the rest of the experiment. |
| 5.0 | Mouse is spontaneously rolling in the cage |
| | (euthanasia is recommended). |
| | - OR - |
| | Mouse is found dead due to paralysis. |
| | - OR - |
| | Mouse is euthanized due to severe paralysis. |

In the recovery stage of EAE, most mice will have a tail that is no longer limp but is not normal either; it feels rigid and is “hooked”. The hind legs may start moving (pedaling), but the mouse cannot walk. Either change makes scoring difficult. Therefore, if this is the case, the following modifications to the above scoring criteria are used for these mice:

TABLE 9
Mouse EAE scoring - modified

| Score | Clinical observations |
|---|---|
| 0.0 | When held by the base of tail, tail is somewhat “hooked” and |
| | rigid, but tail makes complete rotations around the body axis |
| | (“helicopter”). Mouse is healthy. No signs of wobbling. |
| 0.5 | Mouse appears normal but tail is “hooked” and rigid. Tail does not |
| | make complete rotations around the body axis (“helicopter”). |
| | Mouse is healthy. No signs of wobbling. |
| 3.0 | Mouse is found on its side (as described for score 3.5 above), |
| | but there is excessive hind leg movement. Mouse cannot walk. |
| | - OR - |
| | Mouse has a wobbly walk (as described for score 2.5 above), and |
| | is unable to take more than two steps without falling on its side. |
| | The mouse is unable to right itself. |
| | - OR - |
| | Mouse has poor movement in the hind legs (as described for score |
| | 2.5 above), and has partial front leg paralysis evidenced by head |
| | held lower than normal and mouse's inability to right |
| | itself when placed on its side. |
| All | Subtract 0.5 from the score of all mice with either a rigid, |
| other | “hooked” tail or pedaling of hind legs. |
| scores | |

Body Weight

During the course of EAE, changes in body weight reflect disease severity. Mice often lose a small amount of weight on the day following immunization. This appears to be due to effects of the administered adjuvant and pertussis toxin. Mice then steadily increase their body weight until disease onset. On the day of EAE onset, mice consistently lose 1-2 g of their body weight (5-10% of body weight). The weight loss continues with the progression of EAE severity, with the loss reaching around 20% of their pre-onset body weight at the peak of disease. The weight loss is most likely due to both paralysis and reduced food intake as well as high production of pro-inflammatory cytokines such as TNF during the acute phase of inflammation. After the peak of disease is reached, mice slowly gain weight, even if their clinical score does not improve. This increase in weight may be due to down regulation of inflammation which results in lower levels of pro-inflammatory cytokines in blood. Untreated or vehicle-treated mice usually have around 90% of their pre-immunization body weight 28 days after immunization.

Histology

Histological analysis is performed either at the end of the study (usually around 28 days after immunization) or at the time when the vehicle group reaches peak of disease (usually 14-18 days after immunization). Inflammation in EAE normally starts in the lumbar region of the spinal cord, spreading to the entire spinal cord by the peak of disease.

At onset of disease, the number of inflammatory foci correlates strongly with disease severity. The number of foci increases somewhat until the peak of disease, when 6-15 inflammatory foci/section are typically found throughout the spinal cord. In the chronic stage of EAE (starting several days after the peak of disease), many inflammatory foci resolve, typically resulting in 3-4 inflammatory foci in each spinal cord section by approximately 28 days after immunization.

Because the largest numbers of inflammatory foci are present early in the course of disease, if histological analysis is performed at the end of the study, mice which have late EAE onset often have more inflammatory foci in their spinal cords than might be expected from their clinical score. For example, in a 28 day study a mouse with EAE onset on 27 days after immunization and an end clinical score of 2 will likely have more inflammatory foci than a mouse with EAE onset 9 days after immunization and an end score of 3 5 Similarly, a mouse which relapses shortly before the end of the study (relapse is defined as 1 or more points of increase in clinical score) will usually have more inflammatory foci at the end of the study than a mouse with stable chronic disease, even if the two have the same clinical score at the end of the study.

Demyelination is usually not found during the first two days after disease onset, but is found at the peak of disease (4-5 days after EAE onset) and continues during the chronic phase of EAE. Demyelination scores do not change much between the peak and 28 days after immunization and usually average between 1.2 and 2.5.

Demyelination is scored in both Luxol fast blue stained sections (LFB) and in H&E sections. In LFB sections, spinal cord white matter stains dark blue and demyelinated areas are a lighter blue color, and are associated with large vacuoles. In H&E stained sections disruption of normal structure with large vacuoles is indicative of demyelination.

Apoptotic cells are identified in H&E sections, and are usually not found during the first two days of disease development. They are found at the peak and during the chronic stage of EAE. The average number of apoptotic cells is usually between 2 and 4 per section.

Administration of the erythroid cells including HSP60-HLA-E-GPA fusion protein will lead to one or more of an improvement in EAE scoring or a reduction in the number of inflammatory foci compared to vehicle treated control mice.

1. An artificial antigen presenting cell (aAPC) comprising an engineered enucleated erythroid cell comprising an exogenous antigen-presenting polypeptide on the cell surface, wherein the exogenous antigen-presenting polypeptide comprises a human leukocyte antigen-E (HLA-E) polypeptide, and an exogenous antigenic polypeptide that is specifically bound to the exogenous antigen-presenting polypeptide.

2. The aAPC of claim 1, wherein the HLA-E polypeptide comprises an allele selected from the group consisting of: E*01:01:01:01, E*01:01:01:02, E*01:01:01:03, E*01:01:01:04, E*01:01:01:05, E*01:01:01:06, E*01:01:01:07, E*01:01:01:08, E*01:01:01:09, E*01:01:01:10, E*01:01:02, E*01:03:01:01, E*01:03:01:02, E*01:03:01:03, E*01:03:01:04, E*01:03:02:01, E*01:03:02:02, E*01:03:03, E*01:03:04, E*01:03:05, E*01:04, E*01:05, E*01:06, E*01:07, E*01:08N, E*01:09 and E*01:10.

3. (canceled)

4. The aAPC of claim 1, wherein the exogenous antigenic polypeptide comprises a self-peptide, a tolerogenic polypeptide, or an autoimmune disease antigen.

5-7. (canceled)

8. The aAPC of claim 1, wherein the exogenous antigenic polypeptide comprises a polypeptide listed in Table 1.

9. The aAPC of claim 1, wherein the HLA-E polypeptide comprises one or more HLA-E α domains and a β2M polypeptide, or a fragment thereof.

10. The aAPC of claim 9, wherein the HLA-E polypeptide is linked to a membrane anchor.

11. The aAPC of claim 9, wherein the HLA-E polypeptide comprises a single chain fusion protein comprising the exogenous antigenic polypeptide linked to the HLA-E polypeptide via a linker.

12.-17. (canceled)

18. The aAPC of claim 1, wherein the engineered enucleated erythroid cell further comprises an exogenous T regulatory costimulatory polypeptide, an exogenous costimulatory polypeptide, or an exogenous coinhibitory polypeptide on the cell surface.

19-23. (canceled)

24. A method for activating a T regulatory (Treg) cell, the method comprising contacting the Treg cell with the aAPC of claim 1, thereby activating the Treg cell.

25. A method of inhibiting an immune cell, the method comprising contacting the immune cell with the aAPC of claim 1, thereby inhibiting the immune cell.

26. (canceled)

27. A method of treating a subject in need of a modulated immune response, the method comprising contacting an immune cell of the subject with the aAPC of claim 1, thereby treating the subject in need of a modulated immune response.

28-29. (canceled)

30. A method of treating a subject having an autoimmune disease or inflammatory disease, the method comprising:
a) selecting an artificial antigen presenting cell (aAPC), wherein the aAPC is a engineered enucleated erythroid cell comprising an antigen-presenting polypeptide and at least one exogenous antigenic polypeptide that is specifically bound to the antigen-presenting polypeptide, wherein the antigen-presenting polypeptide comprises an HLA-E polypeptide, and
b) administering the aAPC to the subject,
thereby treating the subject having an autoimmune disease or inflammatory disease.

31-32. (canceled)

33. The method of claim 30, wherein the autoimmune disease is selected from the group consisting of type I diabetes, rheumatoid arthritis, graft versus host disease (GVHD), nephritis, multiple sclerosis, mixed connective tissue disorder, pemphigus vulgaris, bullous pemphigoid, membranous glomerulonephritis, neuromyelitis optica, autoimmune encephalomyelitis, autoimmune hepatitis, chronic inflammatory demyelinating polyradiculoneuropathy, dermatomyositis, giant cell arteritis, granulomatosis with polyangiitis, Kawasaki disease, lupus nephritis, polyarteritis nodosa, pyoderma gangrenosum, spondylarthritis, systemic lupus erythematosus, Takayasus arteritis.

34. (canceled)

35. The method of claim 27, wherein the subject has an allergic disorder or an inflammatory disease.

36. (canceled)

37. The method of claim 30, wherein the inflammatory disease is cardiac inflammatory disease, hepatic inflammatory disease, pancreatic inflammatory disease, inflammatory disease of the skin, and/or inflammatory disease of the gastrointestinal (GI) tract. For example, an inflammatory disease includes, but is not limited to, myocarditis, cardiomyopathy, endocarditis, pericarditis, cirrhosis, asthma (eosinophilic or non-eosinophilic), chronic obstructive pulmonary disease (COPD), asthma and COPD overlap syndrome (ACOS), atopic dermatitis, nasal polyps, an allergic response, chronic bronchitis, emphysema, hypersensitivity pneumonitis, allergic rhinitis, chronic rhinosinusitis with or without nasal polyps, inflammatory bowel disease, irritable bowel syndrome, ileitis, chronic inflammatory intestinal disease, fibrosis, eosinophilic esophagitis, vasculitis, urticaria, Churg Strauss syndrome, and inflammatory pain.

38. A method of expanding a population of regulatory T (Treg) cells, the method comprising:
obtaining a population of cells from a subject, wherein the population comprises a Treg cell,
contacting the population with the aAPC of claim 1, wherein contacting the population with the aAPC induces proliferation of the Treg cell,
thereby expanding the population of Treg cells.

39-40. (canceled)

41. A method of making the aAPC of claim 1, the method comprising:
introducing an exogenous nucleic acid encoding the exogenous antigen-presenting polypeptide into a nucleated erythroid precursor cell; and
culturing the nucleated erythroid precursor cell under conditions suitable for enucleation and for production of the exogenous antigen-presenting polypeptide, thereby making the aAPC.

42-48. (canceled)

49. An artificial antigen presenting cell (aAPC) comprising an engineered enucleated erythroid cell comprising an exogenous antigen-presenting polypeptide on the cell surface, wherein the exogenous antigen-presenting polypeptide comprises a human leukocyte antigen-G (HLA-G) polypeptide, and an exogenous antigenic polypeptide that is specifically bound to the exogenous antigen-presenting polypeptide.

50. The aAPC of claim 49, wherein the HLA-G polypeptide is selected from the group consisting of: HLA-G1, HLA-G2, HLA-G3, HLA-G4, HLA-G5, HLA-G6, and HLA-G7.

51. (canceled)

52. The aAPC of claim 49, wherein the exogenous antigenic polypeptide comprises the motif XI/LPXXXXXL (SEQ ID NO: 8).

53. The aAPC of claim 49, wherein the HLA-G polypeptide comprises one or more HLA-G a domains and a β2M polypeptide, or a fragment thereof.

54. The aAPC of claim 53, wherein the HLA-G polypeptide is linked to a membrane anchor.

55. The aAPC of claim 53, wherein the HLA-G polypeptide comprises a single chain fusion protein comprising Drill the exogenous antigenic polypeptide linked to the HLA-G polypeptide via a linker.

56-74. (canceled)

75. A method of suppressing activity of an immune cell, the method comprising contacting the immune cell with the aAPC of claim 49, thereby suppressing activity of the immune cell.

76. The method of claim 75, wherein the immune cell is selected from the group consisting of a T cell, B cell, NK cell, macrophage, and dendritic cell.

77-81. (canceled)

82. A method of treating a subject in need of a reduced immune response, the method comprising contacting an immune cell of the subject with the aAPC of claim 49, thereby treating the subject in need of a reduced immune response.

83. The method of claim 82, wherein the subject has an autoimmune disease, an inflammatory disease or an allergic disease, or wherein the subject is in need of or has undergone a transplantation.

84. The method of claim 83, wherein the autoimmune disease is selected from the group consisting of type I diabetes, rheumatoid arthritis, GVHD, nephritis and multiple sclerosis.

85-87. (canceled)

88. A method of making the aAPC of claim 49, the method comprising:
introducing an exogenous nucleic acid encoding the exogenous antigenic polypeptide into a nucleated erythroid precursor cell; and
culturing the nucleated erythroid precursor cell under conditions suitable for enucleation and production of the exogenous antigenic polypeptide, thereby making an engineered enucleated erythroid cell,
thereby making the aAPC.

89-95. (canceled)


SEQUENCE LISTING (130 sequences)

SEQ ID NO: 1 - protein, 9 aa, Artificial Sequence
  Description of Artificial Sequence Synthetic peptide
YPYDVPDYA

SEQ ID NO: 2 - protein, 10 aa, Artificial Sequence
  Description of Artificial Sequence Synthetic peptide
EQKLISEEDL

SEQ ID NO: 3 - protein, 8 aa, Artificial Sequence
  Description of Artificial Sequence Synthetic peptide
DYKDDDDK

SEQ ID NO: 4 - DNA, 54 bp, Artificial Sequence
  Description of Artificial Sequence Synthetic oligonucleotide
gagggcagaggaagtcttctaacatgcggtgacgtggaggsgsstcccggccct

SEQ ID NO: 5 - protein, 15 aa, Artificial Sequence
  Description of Artificial Sequence Synthetic peptide
GGGGSGGGGSGGGGS

SEQ ID NO: 6 - protein, 5 aa, Artificial Sequence
  Description of Artificial Sequence Synthetic peptide
  MOD_RES (3)..(3): Any amino acid
LPXTG

SEQ ID NO: 7 - protein, 5 aa, Artificial Sequence
  Description of Artificial Sequence Synthetic peptide
  MOD_RES (3)..(3): Any amino acid
LPXTA

SEQ ID NO: 8 - protein, 9 aa, Artificial Sequence
  Description of Artificial Sequence Synthetic peptide
  MOD_RES (1)..(1): Any amino acid
  MOD_RES (2)..(2): Ile or Leu
  MOD_RES (4)..(8): Any amino acid
XXPXXXXXL

SEQ ID NO: 9 - protein, 10 aa, Artificial Sequence
  Description of Artificial Sequence Synthetic peptide
FENDAQAPKS

SEQ ID NO: 10 - protein, 10 aa, Artificial Sequence
  Description of Artificial Sequence Synthetic peptide
LQNDAQAPKS

SEQ ID NO: 11 - protein, 9 aa, Artificial Sequence
  Description of Artificial Sequence Synthetic peptide
SQQPYLQLQ

SEQ ID NO: 12 - protein, 9 aa, Artificial Sequence
  Description of Artificial Sequence Synthetic peptide
ALALVRMLI

SEQ ID NO: 13 - protein, 10 aa, Artificial Sequence
  Description of Artificial Sequence Synthetic peptide
YLLPRRGPRL

SEQ ID NO: 14 - protein, 9 aa, Artificial Sequence
  Description of Artificial Sequence Synthetic peptide
AISPRTLNA

SEQ ID NO: 15 - protein, 9 aa, Artificial Sequence
  Description of Artificial Sequence Synthetic peptide
VMAPRTLIL

SEQ ID NO: 16 - protein, 9 aa, Artificial Sequence
  Description of Artificial Sequence Synthetic peptide
SQAPLPCVL

SEQ ID NO: 17 - protein, 9 aa, Artificial Sequence
  Description of Artificial Sequence Synthetic peptide
QMRPVSRVL

SEQ ID NO: 18 - protein, 17 aa, Artificial Sequence
  Description of Artificial Sequence Synthetic peptide
HAVSEGTKAVTKYTSSK

SEQ ID NO: 19 - protein, 17 aa, Artificial Sequence
  Description of Artificial Sequence Synthetic peptide
PAETATPAPVEKSPAKK

SEQ ID NO: 20 - protein, 17 aa, Artificial Sequence
  Description of Artificial Sequence Synthetic peptide
AYVRLAPDYDALDVANK

SEQ ID NO: 21 - protein, 17 aa, Artificial Sequence
  Description of Artificial Sequence Synthetic peptide
HAVSEGTKAVTKYTSAK

SEQ ID NO: 22 - protein, 17 aa, Artificial Sequence
  Description of Artificial Sequence Synthetic peptide
AVSDGVIKVFNDMKVRK

SEQ ID NO: 23 - protein, 16 aa, Artificial Sequence
  Description of Artificial Sequence Synthetic peptide
HAVSEGTKAVTKYTSS

SEQ ID NO: 24 - protein, 16 aa, Artificial Sequence
  Description of Artificial Sequence Synthetic peptide
QLLQANPILEAFGNAK

SEQ ID NO: 25 - protein, 16 aa, Artificial Sequence
  Description of Artificial Sequence Synthetic peptide
KSADTLWDIQKDLKDL

SEQ ID NO: 26 - protein, 16 aa, Artificial Sequence
  Description of Artificial Sequence Synthetic peptide
AYVRLAPDYDALDVAN

SEQ ID NO: 27 - protein, 16 aa, Artificial Sequence
  Description of Artificial Sequence Synthetic peptide
HAVSEGTKAVTKYTSA

SEQ ID NO: 28 - protein, 16 aa, Artificial Sequence
  Description of Artificial Sequence Synthetic peptide
TGLIKGSGTAEVELKK

SEQ ID NO: 29 - protein, 16 aa, Artificial Sequence
  Description of Artificial Sequence Synthetic peptide
VSDGVIKVFNDMKVRK

SEQ ID NO: 30 - protein, 16 aa, Artificial Sequence
  Description of Artificial Sequence Synthetic peptide
ASGNYATVISHNPETK

SEQ ID NO: 31 - protein, 16 aa, Artificial Sequence
  Description of Artificial Sequence Synthetic peptide
TAEILELAGNAARDNK

SEQ ID NO: 32 - protein, 16 aa, Artificial Sequence
  Description of Artificial Sequence Synthetic peptide
HAVSEGTKAVTKYTSA

SEQ ID NO: 33 - protein, 15 aa, Artificial Sequence
  Description of Artificial Sequence Synthetic peptide
PAPVEKSPAKKKATK

SEQ ID NO: 34 - protein, 15 aa, Artificial Sequence
  Description of Artificial Sequence Synthetic peptide
SADTLWDIQKDLKDL

SEQ ID NO: 35 - protein, 15 aa, Artificial Sequence
  Description of Artificial Sequence Synthetic peptide
TGLIKGSGTAEVELK

SEQ ID NO: 36 - protein, 15 aa, Artificial Sequence
  Description of Artificial Sequence Synthetic peptide
KSADTLWGIQKELQF

SEQ ID NO: 37 - protein, 15 aa, Artificial Sequence
  Description of Artificial Sequence Synthetic peptide
HGSYEDAVHSGALND

SEQ ID NO: 38 - protein, 15 aa, Artificial Sequence
  Description of Artificial Sequence Synthetic peptide
SDGVIKVFNDMKVRK

SEQ ID NO: 39 - protein, 15 aa, Artificial Sequence
  Description of Artificial Sequence Synthetic peptide
AGNLGGGVVTIERSK

SEQ ID NO: 40 - protein, 15 aa, Artificial Sequence
  Description of Artificial Sequence Synthetic peptide
AQAAAPASVPAQAPK

SEQ ID NO: 41 - protein, 15 aa, Artificial Sequence
  Description of Artificial Sequence Synthetic peptide
PRKIEEIKDFLLTAR

SEQ ID NO: 42 - protein, 14 aa, Artificial Sequence
  Description of Artificial Sequence Synthetic peptide
SEGTKAVTKYTSSK

SEQ ID NO: 43 - protein, 14 aa, Artificial Sequence
  Description of Artificial Sequence Synthetic peptide
VLKQVHPDTGISSK

SEQ ID NO: 44 - protein, 14 aa, Artificial Sequence
  Description of Artificial Sequence Synthetic peptide
SWTAADTAAQITQR

SEQ ID NO: 45 - protein, 14 aa, Artificial Sequence
  Description of Artificial Sequence Synthetic peptide
FISVGYVDDTQFVR

SEQ ID NO: 46 - protein, 14 aa, Artificial Sequence
  Description of Artificial Sequence Synthetic peptide
NIDDGTSDRPYSHA

SEQ ID NO: 47 - protein, 14 aa, Artificial Sequence
  Description of Artificial Sequence Synthetic peptide
VLKQVHPDTGISSK

SEQ ID NO: 48 - protein, 14 aa, Artificial Sequence
  Description of Artificial Sequence Synthetic peptide
RKTVTAMDVVYALK

SEQ ID NO: 49 - protein, 14 aa, Artificial Sequence
  Description of Artificial Sequence Synthetic peptide
SADTLWGIQKELQF

SEQ ID NO: 50 - protein, 14 aa, Artificial Sequence
  Description of Artificial Sequence Synthetic peptide
ASAETVDPASLWEY

SEQ ID NO: 51 - protein, 13 aa, Artificial Sequence
  Description of Artificial Sequence Synthetic peptide
TVVNKDVFRDPAL

SEQ ID NO: 52 - protein, 13 aa, Artificial Sequence
  Description of Artificial Sequence Synthetic peptide
KTVTAMDVVYALK

SEQ ID NO: 53 - protein, 13 aa, Artificial Sequence
  Description of Artificial Sequence Synthetic peptide
EGIPALDNFLDKL

SEQ ID NO: 54 - protein, 13 aa, Artificial Sequence
  Description of Artificial Sequence Synthetic peptide
RVTIMPKDIQLAR

SEQ ID NO: 55 - protein, 13 aa, Artificial Sequence
  Description of Artificial Sequence Synthetic peptide
PVAVMAESAFSFK

SEQ ID NO: 56 - protein, 13 aa, Artificial Sequence
  Description of Artificial Sequence Synthetic peptide
QTVAVGVIKAVDK

SEQ ID NO: 57 - protein, 13 aa, Artificial Sequence
  Description of Artificial Sequence Synthetic peptide
ILELAGNAARDNK

SEQ ID NO: 58 - protein, 12 aa, Artificial Sequence
  Description of Artificial Sequence Synthetic peptide
GTGASGSFKLNK

SEQ ID NO: 59 - protein, 12 aa, Artificial Sequence
  Description of Artificial Sequence Synthetic peptide
KQVHPDTGISSK

SEQ ID NO: 60 - protein, 12 aa, Artificial Sequence
  Description of Artificial Sequence Synthetic peptide
VGGTSDVEVNEK

SEQ ID NO: 61 - protein, 12 aa, Artificial Sequence
  Description of Artificial Sequence Synthetic peptide
NSVVEASEAAYK

SEQ ID NO: 62 - protein, 12 aa, Artificial Sequence
  Description of Artificial Sequence Synthetic peptide
ALRYPMAVGLNK

SEQ ID NO: 63 - protein, 12 aa, Artificial Sequence
  Description of Artificial Sequence Synthetic peptide
SLVSKGTLVQTK

SEQ ID NO: 64 - protein, 11 aa, Artificial Sequence
  Description of Artificial Sequence Synthetic peptide
PELAKSAPAPK

SEQ ID NO: 65 - protein, 11 aa, Artificial Sequence
  Description of Artificial Sequence Synthetic peptide
SEMEVQDAELK

SEQ ID NO: 66 - protein, 11 aa, Artificial Sequence
  Description of Artificial Sequence Synthetic peptide
QTYSTEPNNLK

SEQ ID NO: 67 - protein, 11 aa, Artificial Sequence
  Description of Artificial Sequence Synthetic peptide
PMFIVNTNVPR

SEQ ID NO: 68 - protein, 10 aa, Artificial Sequence
  Description of Artificial Sequence Synthetic peptide
AGFAGDDAPR

SEQ ID NO: 69 - protein, 10 aa, Artificial Sequence
  Description of Artificial Sequence Synthetic peptide
RVNAGTLAVL

SEQ ID NO: 70 - protein, 9 aa, Artificial Sequence
  Description of Artificial Sequence Synthetic peptide
IGQSKVFFR

SEQ ID NO: 71 - protein, 16 aa, Artificial Sequence
  Description of Artificial Sequence Synthetic peptide
TAEILELAGNAARDNK

SEQ ID NO: 72 - protein, 13 aa, Artificial Sequence
  Description of Artificial Sequence Synthetic peptide
ILELAGNAARDNK

SEQ ID NO: 73 - protein, 17 aa, Artificial Sequence
  Description of Artificial Sequence Synthetic peptide
ALAGCHLEDTQRKLQKG

SEQ ID NO: 74 - protein, 16 aa, Artificial Sequence
  Description of Artificial Sequence Synthetic peptide
MQLITRGKGAGTPNLI

SEQ ID NO: 75 - protein, 15 aa, Artificial Sequence
  Description of Artificial Sequence Synthetic peptide
KMKLRNTVHLSYLTV

SEQ ID NO: 76 - protein, 15 aa, Artificial Sequence
  Description of Artificial Sequence Synthetic peptide
CRASQTISSYLDWYQ

SEQ ID NO: 77 - protein, 13 aa, Artificial Sequence
  Description of Artificial Sequence Synthetic peptide
PAALTNKGNTVFA

SEQ ID NO: 78 - protein, 13 aa, Artificial Sequence
  Description of Artificial Sequence Synthetic peptide
WTPGPSAGVTGIA

SEQ ID NO: 79 - protein, 10 aa, Artificial Sequence
  Description of Artificial Sequence Synthetic peptide
ILRTIGKEAF

SEQ ID NO: 80 - protein, 9 aa, Artificial Sequence
  Description of Artificial Sequence Synthetic peptide
RSCGYACTA

SEQ ID NO: 81 - protein, 9 aa, Artificial Sequence
  Description of Artificial Sequence Synthetic peptide
FPNGFSFIH

SEQ ID NO: 82 - protein, 9 aa, Artificial Sequence
  Description of Artificial Sequence Synthetic peptide
SHGPYIKLI

SEQ ID NO: 83 - protein, 15 aa, Artificial Sequence
  Description of Artificial Sequence Synthetic peptide
AQAAAPASVPAQAPK

SEQ ID NO: 84 - protein, 17 aa, Artificial Sequence
  Description of Artificial Sequence Synthetic peptide
AYVRLAPDYDALDVANK

SEQ ID NO: 85 - protein, 16 aa, Artificial Sequence
  Description of Artificial Sequence Synthetic peptide
AYVRLAPDYDALDVAN

SEQ ID NO: 86 - protein, 16 aa, Artificial Sequence
  Description of Artificial Sequence Synthetic peptide
ASGNYATVISHNPETK

SEQ ID NO: 87 - protein, 15 aa, Artificial Sequence
  Description of Artificial Sequence Synthetic peptide
AGNLGGGVVTIERSK

SEQ ID NO: 88 - protein, 15 aa, Artificial Sequence
  Description of Artificial Sequence Synthetic peptide
PRKIEEIKDFLLTAR

SEQ ID NO: 89 - protein, 14 aa, Artificial Sequence
  Description of Artificial Sequence Synthetic peptide
NIDDGTSDRPYSHA

SEQ ID NO: 90 - protein, 13 aa, Artificial Sequence
  Description of Artificial Sequence Synthetic peptide
TVVNKDVFRDPAL

SEQ ID NO: 91 - protein, 12 aa, Artificial Sequence
  Description of Artificial Sequence Synthetic peptide
ALRYPMAVGLNK

SEQ ID NO: 92 - protein, 11 aa, Artificial Sequence
  Description of Artificial Sequence Synthetic peptide
QTYSTEPNNLK

SEQ ID NO: 93 - protein, 11 aa, Artificial Sequence
  Description of Artificial Sequence Synthetic peptide
PELAKSAPAPK

SEQ ID NO: 94 - protein, 12 aa, Artificial Sequence
  Description of Artificial Sequence Synthetic peptide
KQVHPDTGISSK

SEQ ID NO: 95 - protein, 14 aa, Artificial Sequence
  Description of Artificial Sequence Synthetic peptide
VLKQVHPDTGISSK

SEQ ID NO: 96 - protein, 14 aa, Artificial Sequence
  Description of Artificial Sequence Synthetic peptide
SEGTKAVTKYTSSK

SEQ ID NO: 97 - protein, 16 aa, Artificial Sequence
  Description of Artificial Sequence Synthetic peptide
HAVSEGTKAVTKYTSA

SEQ ID NO: 98 - protein, 16 aa, Artificial Sequence
  Description of Artificial Sequence Synthetic peptide
HAVSEGTKAVTKYTSS

SEQ ID NO: 99 - protein, 17 aa, Artificial Sequence
  Description of Artificial Sequence Synthetic peptide
HAVSEGTKAVTKYTSAK

SEQ ID NO: 100 - protein, 17 aa, Artificial Sequence
  Description of Artificial Sequence Synthetic peptide
HAVSEGTKAVTKYTSSK

SEQ ID NO: 101 - protein, 16 aa, Artificial Sequence
  Description of Artificial Sequence Synthetic peptide
HAVSEGTKAVTKYTSA

SEQ ID NO: 102 - protein, 10 aa, Artificial Sequence
  Description of Artificial Sequence Synthetic peptide
AGFAGDDAPR

SEQ ID NO: 103 - protein, 9 aa, Artificial Sequence
  Description of Artificial Sequence Synthetic peptide
SQAPLPCVL

SEQ ID NO: 104 - protein, 9 aa, Artificial Sequence
  Description of Artificial Sequence Synthetic peptide
VMAPRTLFL

SEQ ID NO: 105 - protein, 13 aa, Artificial Sequence
  Description of Artificial Sequence Synthetic peptide
PKKTESHHKAKGK

SEQ ID NO: 106 - protein, 7 aa, Artificial Sequence
  Description of Artificial Sequence Synthetic peptide
AAVLEYL

SEQ ID NO: 107 - protein, 25 aa, Artificial Sequence
  Description of Artificial Sequence Synthetic peptide
AQAAAPASVPAQAPKRTQAPTKASE

SEQ ID NO: 108 - protein, 19 aa, Artificial Sequence
  Description of Artificial Sequence Synthetic peptide
KLEKEEEEGISQESSEEEQ

SEQ ID NO: 109 - protein, 19 aa, Artificial Sequence
  Description of Artificial Sequence Synthetic peptide
GDRSEDFGVNEDLADSDAR

SEQ ID NO: 110 - protein, 18 aa, Artificial Sequence
  Description of Artificial Sequence Synthetic peptide
VAPEEHPVLLTEAPLNPK

SEQ ID NO: 111 - protein, 16 aa, Artificial Sequence
  Description of Artificial Sequence Synthetic peptide
STAGDTHLGGEDFDNR

SEQ ID NO: 112 - protein, 15 aa, Artificial Sequence
  Description of Artificial Sequence Synthetic peptide
KVPQVSTPTLVEVSR

SEQ ID NO: 113 - protein, 15 aa, Artificial Sequence
  Description of Artificial Sequence Synthetic peptide
PDPAKSAPAPKKGSK

SEQ ID NO: 114 - protein, 12 aa, Artificial Sequence
  Description of Artificial Sequence Synthetic peptide
LQAEIEGLKGQR

SEQ ID NO: 115 - protein, 11 aa, Artificial Sequence
  Description of Artificial Sequence Synthetic peptide
PDPAKSAPAPK

SEQ ID NO: 116 - protein, 11 aa, Artificial Sequence
  Description of Artificial Sequence Synthetic peptide
PELAKSAPAPK

SEQ ID NO: 117 - protein, 11 aa, Artificial Sequence
  Description of Artificial Sequence Synthetic peptide
PEPVKSAPVPK

SEQ ID NO: 118 - protein, 9 aa, Artificial Sequence
  Description of Artificial Sequence Synthetic peptide
AAPATRAAL

SEQ ID NO: 119 - protein, 9 aa, Artificial Sequence
  Description of Artificial Sequence Synthetic peptide
SAPSRATAL

SEQ ID NO: 120 - protein, 8 aa, Artificial Sequence
  Description of Artificial Sequence Synthetic peptide
ILNFPPPP

SEQ ID NO: 121 - protein, 8 aa, Artificial Sequence
  Description of Artificial Sequence Synthetic peptide
IAPTGHSL

SEQ ID NO: 122 - protein, 8 aa, Artificial Sequence
  Description of Artificial Sequence Synthetic peptide
ISPHGNAL

SEQ ID NO: 123 - protein, 15 aa, Artificial Sequence
  Description of Artificial Sequence Synthetic peptide
PDPAKSAPAPKKGSK

SEQ ID NO: 124 - protein, 11 aa, Artificial Sequence
  Description of Artificial Sequence Synthetic peptide
PDPAKSAPAPK

SEQ ID NO: 125 - protein, 11 aa, Artificial Sequence
  Description of Artificial Sequence Synthetic peptide
PELAKSAPAPK

SEQ ID NO: 126 - protein, 11 aa, Artificial Sequence
  Description of Artificial Sequence Synthetic peptide
PEPVKSAPVPK

SEQ ID NO: 127 - protein, 18 aa, Artificial Sequence
  Description of Artificial Sequence Synthetic peptide
VAPEEHPVLLTEAPLNPK

SEQ ID NO: 128 - protein, 9 aa, Artificial Sequence
  Description of Artificial Sequence Synthetic peptide
RMPPLGHEL

SEQ ID NO: 129 - protein, 9 aa, Artificial Sequence
  Description of Artificial Sequence Synthetic peptide
VLRPGGHFL

SEQ ID NO: 130 - protein, 9 aa, Artificial Sequence
  Description of Artificial Sequence Synthetic peptide
VMATRRNVL